(19) United States
(12) Patent Application Publication
(10) Pub. No.: US 2023/0220095 A1
(43) Pub. Date: Jul. 13, 2023

(54) COMPOSITIONS AND METHODS FOR TREATMENT OF CANCER

(71) Applicant: Aleta Biotherapeutics Inc., Natick, MA (US)

(72) Inventors: Roy Lobb, Wellesley, MA (US); Paul Rennert, Holliston, MA (US)

(21) Appl. No.: 17/909,315

(22) PCT Filed: Mar. 8, 2021
(86) PCT No.: PCT/US2021/021363

§ 371 Date: Sep. 2, 2022

Related U.S. Application Data
(60) Provisional application No. 62/986,310, filed on Mar. 6, 2020.

(51) Int. Cl. C07K 16/28; C07K 14/47; C07K 14/725; A61P 35/00

(52) U.S. Cl. CPC C07K 16/2878; A61P 35/00; C07K 14/4748; C07K 14/7051; C07K 16/2896; C07K 2317/31; C07K 2317/73; C07K 2317/92; C07K 2317/569; C07K 2317/622; C07K 2319/03; C07K 2319/30

(57) ABSTRACT

Compositions, e.g., compositions comprising protein therapeutics, and methods of using such compositions for treating cancer are described.

### CROSS REFERENCE TO RELATED APPLICATIONS

This application claims priority to U.S. Provisional Pat. Application No. 62/986,310, filed on Mar. 6, 2020, the entire contents of which are hereby incorporated by reference.

### BACKGROUND

Adoptive cell therapy (ACT) is a treatment method in which cells are removed from a donor, cultured and/or manipulated ex vivo, and then administered to a patient for the treatment of a disease. A variety of cell types have been used in ACT in an attempt to treat several classes of disorders. For the treatment of cancer, ACT generally involves the transfer of lymphocytes, such as chimeric antigen receptor (CAR) T cells. However, subjects receiving ACT can relapse. Accordingly, there remains a need for improved methods for treating cancer using adoptive cell therapy.

### SUMMARY

The present disclosure provides methods and compositions useful for treatment of cancer and/or for initiating or modulating immune responses. In some embodiments, the present invention provides methods and compositions useful for initial treatment of cancers. In some embodiments, the present invention provides methods and compositions useful for treatment of cancers following relapse. In some embodiments the present invention provides methods and compositions useful for treatment of multiple myeloma.

In some embodiments the present disclosure provides a method of treating a subject suffering from cancer, the method comprising administering to the subject a fusion protein comprising an antigen binding polypeptide and a polypeptide antigen to the subject, thereby treating the subject, wherein (i) the subject previously received and/or is receiving adoptive cell therapy (ACT), (ii) the subject previously exhibited at least one beneficial response to the ACT, and (iii) prior to the administration of the fusion protein, the subject exhibits at least one nonbeneficial response to the ACT. In some embodiments, ACT comprises administering a cell selected from the group consisting of NK cells, tumor-infiltrating lymphocytes (TIL), autologous or allogeneic CAR-T cells, myeloid-derived cells, Induced pluripotent stem cells (IPSC), gamma delta T cells, invariant NK cells, NK-T cells and other useful cell types. In some embodiments, the fusion protein comprises 2 or more antigen binding polypeptides.

In some embodiments, a beneficial response comprises clearance, regression, and/or or stabilization of the cancer, e.g., over a defined period of time (e.g., at least 1, 2, 3, 4, 5, 6, 7, 8, 9, 10, 11, 12 weeks, or at least 1, 2, 3, 4, 5, 6, 7, 8, 9, 10, 11, 12 months, or at least 1, 2, 3, 4, 5, 6, 7, 8, 9, 10, 11, 12 years). In some embodiments, a beneficial response comprises an absence of relapse, recurrence, and/or metastasis of the cancer, e.g., over a defined period of time (e.g., at least 1, 2, 3, 4, 5, 6, 7, 8, 9, 10, 11, 12 weeks, or at least 1, 2, 3, 4, 5, 6, 7, 8, 9, 10, 11, 12 months, or at least 1, 2, 3, 4, 5, 6, 7, 8, 9, 10, 11, 12 years). In some embodiments, a nonbeneficial response comprises a relapse, recurrence, and/or metastasis of the cancer.

In some embodiments, prior to administration of a fusion protein, a measured level of expression of an ACT target antigen is reduced relative to a control level (e.g., a level of expression of the target antigen in a subject exhibiting at least one beneficial response to the ACT; and/or the level of expression of the target antigen in the subject during a period in which the subject previously exhibited a beneficial response to the ACT).

In some embodiments the present disclosure provides a method of treating a subject who previously received and/or is receiving ACT comprising a cell that binds a target antigen, the method comprising: administering to the subject a fusion protein if a level of expression of the target antigen in a sample from the subject (e.g., a biological sample, e.g., a tumor sample) is reduced relative to a control level (e.g., a level of expression of the target antigen in a subject exhibiting at least one beneficial response to the ACT; and/or the level of expression of the target antigen in the subject during a period in which the subject previously exhibited a beneficial response to the ACT), wherein the fusion protein comprises an antigen binding polypeptide and a polypeptide antigen, thereby treating the subject.

In some embodiments, the present disclosure provides, a method of selecting a subject for treatment with ACT comprising a cell that binds a target antigen, the method comprising: measuring a level of expression of the target antigen in a sample from the subject (e.g., a biological sample, e.g., a tumor sample); comparing the level of expression to a control level (e.g., a level of expression of the target antigen in a subject exhibiting at least one beneficial response to the ACT; and/or the level of expression of the target antigen in the subject during a period in which the subject previously exhibited a beneficial response to the ACT); and selecting the subject for treatment with the ACT and a fusion protein if the level of expression of the target antigen is reduced relative to the control level, wherein the fusion protein comprises an antigen binding polypeptide and a polypeptide antigen.

In some embodiments, the present disclosure provides, methods of treating a subject having or suffering from multiple myeloma, the method comprising: administering to the subject a fusion protein comprising: (a) an antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and (b) a polypeptide antigen comprising a second multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the first multiple myeloma antigen and the second multiple myeloma antigen are different; wherein the subject is receiving or will receive ACT (e.g., CAR-T cell therapy) for the treatment of multiple myeloma.

In some embodiments, fusion proteins of the present disclosure comprise one or more antigen binding polypeptides. In some embodiments, fusion proteins of the present disclosure comprise two or more antigen binding polypeptides. In some embodiments, fusion proteins of the present disclosure comprise one or more antigen binding polypeptides that are the same. In some embodiments, fusion proteins of the present disclosure comprise one or more antigen binding polypeptides that bind the same antigen. In some embodiments, the present disclosure provides a method of treatment, wherein the fusion protein comprises: a first antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; (b) a second antigen binding polypeptide that binds a second multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and (c) a polypeptide antigen comprising of a third multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the third multiple myeloma antigen is different than the first and second. In some embodiments, the first multiple myeloma antigen and the second multiple myeloma antigen are the same. In some embodiments, the first multiple myeloma antigen and the second multiple myeloma antigen are CD38. In some embodiments, the first and second antigen binding polypeptide are the same, and the fusion protein comprises two copies of the same antigen binding polypeptide. In some embodiments, the first and second antigen binding polypeptide bind to the first and second multiple myeloma antigen at a Kd of about 50 nM to about 2 µM. In some embodiments, the fusion protein binds to a tumor cell expressing the first and second multiple myeloma antigen (e.g., CD38) with higher avidity relative to a healthy or non-tumor cell. In some embodiments, the fusion protein binds to the tumor cell at a Kd of about 1 to about 40 nM.

### BRIEF DESCRIPTION OF THE DRAWINGS

FIG. 1 demonstrates low expression of BCMA and high expression of CD38 on Daudi cells.

FIG. 2 demonstrates binding of BCMA-anti-CD38 fusion proteins to Daudi cells as detected by anti-HIS tag antibodies.

FIG. 3 demonstrates binding of BCMA-anti-CD38 fusion proteins to Daudi cells as detected by anti-BCMA antibodies.

FIG. 4 demonstrates expression levels of GPRC5D after transfection of HEK293 cells.

FIG. 5 demonstrates binding of four different anti-GPRC5D-BCMA fusion proteins to GPRC5D expressing HEK293 cells as detected by anti-HIS tag antibodies.

FIG. 6 demonstrates binding of four different anti-GPRC5D-BCMA fusion proteins to GPRC5D expressing HEK293 cells as detected by anti-BCMA antibodies.

FIG. 7 demonstrates that anti-BCMA CAR-T cells, but not untransduced donor-matched T cells (UTD), are capable of killing multiple myeloma cells in culture.

FIGS. 8A-8B demonstrate that anti-BCMA CAR-T cells are capable of killing HEK293 cells expressing GPRC5D only in the presence of anti-GPRC5D-BCMA fusion proteins. FIG. 8A shows CAR-T 397 which binds BCMA kills H929 myeloma cells (positive control) FIG. 8B shows CAR-T 397 which binds BCMA kills BCMA-negative HEK293T cells transiently transfected with GPRC5D only upon addition of a fusion protein comprising an anti-GPRC5D binding polypeptide and a BCMA polypeptide antigen. The fusion protein was added at 500 ng/ml or 100 ng/ml. Notably CAR-T 397 does not kill cells in the absence of fusion protein #538.

FIG. 9 shows exemplary combinations of antigen binding polypeptides that bind to a multiple myeloma antigen and polypeptide antigens.

### DEFINITIONS

In order for the present invention to be more readily understood, certain terms are first defined below. Additional definitions for the following terms and other terms are set forth throughout the specification.

Administration: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (including by intratracheal instillation), transdermal, vaginal and vitreal. In some embodiments, administration may be intratumoral or peritumoral. In some embodiments, administration may involve intermittent dosing. In some embodiments, administration may involve continuous dosing (e.g., perfusion) for at least a selected period of time.

Adoptive cell therapy: As used herein, “adoptive cell therapy” or “ACT” involves the transfer of immune cells with anti-tumor activity into cancer patients. In some embodiments, ACT is a treatment approach that involves the use of lymphocytes with anti-tumor activity, the in vitro expansion of these cells to large numbers and their infusion into a cancer-bearing host.

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Amelioration: As used herein, “amelioration” refers to prevention, reduction and/or palliation of a state, or improvement of the state of a subject. Amelioration includes, but does not require, complete recovery or complete prevention of a disease, disorder or condition.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an l-amino acid. “Standard amino acid” refers to any of the twenty standard l-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide’s circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)- an amino-terminal variable (VH) domain (located at the tips of the Y structure), followed by three constant domains: CH1, CH2, and the carboxy-terminal CH3 (located at the base of the Y’s stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains - an amino-terminal variable (VL) domain, followed by a carboxy-terminal constant (CL) domain, separated from one another by another “switch”. Intact antibody tetramers are composed of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are fully human, or are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgG, IgE and IgM, bi- or multi- specific antibodies (e.g., Zybodies®, etc), bi- or multi-paratopic antibodies, single chain Fvs, polypeptide-Fc fusions, Fabs, camelid antibodies, masked antibodies (e.g., Probodies®), Small Modular ImmunoPharmaceuticals (“SMIPsTM”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.)).

Antibody-Dependent Cellular Cytotoxicity: As used herein, the term “antibody-dependent cellular cytotoxicity” or “ADCC” refers to a phenomenon in which target cells bound by antibody are killed by immune effector cells. Without wishing to be bound by any particular theory, ADCC is typically understood to involve Fc receptor (FcR)-bearing effector cells can recognizing and subsequently killing antibody-coated target cells (e.g., cells that express on their surface specific antigens to which an antibody is bound). Effector cells that mediate ADCC can include immune cells, including but not limited to one or more of natural killer (NK) cells, macrophage, neutrophils, eosinophils.

Antibody Fragment: As used herein, an “antibody fragment” includes a portion of an intact antibody, such as, for example, the antigen-binding or variable region of an antibody. Examples of antibody fragments include Fab, Fab′, F(ab′)₂, and Fv fragments; triabodies; tetrabodies; linear antibodies; single-chain antibody molecules; and multi-specific antibodies formed from antibody fragments. For example, antibody fragments include isolated fragments, “Fv” fragments (consisting of the variable regions of the heavy and light chains), recombinant single chain polypeptide molecules in which light and heavy chain variable regions are connected by a peptide linker (“scFv proteins”), recombinant single domain antibodies consisting of a variable region of an antibody heavy chain (e.g., VHH), and minimal recognition units consisting of the amino acid residues that mimic a hypervariable region (e.g., a hypervariable region of a heavy chain variable region (VH), a hypervariable region of a light chain variable region (VL), one or more CDR domains within the VH, and/or one or more CDR domains within the VL). In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab’)₂ fragment, scFv fragment, Fv fragment, dsFv diabody, dAb fragment, Fd′ fragment, Fd fragment, heavy chain variable region, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment. Alternatively or additionally, an antigen binding fragment of an antibody may comprise multiple chains which are linked together, for example, by disulfide linkages. An antigen binding fragment of an antibody may optionally comprise a multimolecular complex. A functional antibody fragment typically comprises at least about 50 amino acids and more typically comprises at least about 200 amino acids.

Antigen: The term “antigen”, as used herein, refers to an agent that elicits an immune response; and/or an agent that binds to a T cell receptor (e.g., when presented by an MHC molecule) or to an antibody or antibody fragment. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source), or alternatively may exist on or in a cell. In some embodiments, an antigen is a recombinant antigen.

Antigen presenting cell: The phrase “antigen presenting cell” or “APC,” as used herein, has its art understood meaning referring to cells that process and present antigens to T-cells. Exemplary APC include dendritic cells, macrophages, B cells, certain activated epithelial cells, and other cell types capable of TCR stimulation and appropriate T cell costimulation.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts - including where interacting entities or moieties are studied in isolation or in the context of more complex systems (e.g., while covalently or otherwise associated with a carrier entity and/or in a biological system or cell).

Cancer: The terms “cancer”, “malignancy”, “neoplasm”, “tumor”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, hematopoietic cancers including leukemias, lymphomas (Hodgkins and non-Hodgkins), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Combination Therapy: As used herein, the term “combination therapy” refers to those situations in which a subject is simultaneously exposed to two or more therapeutic regimens (e.g., two or more therapeutic agents). In some embodiments, two or more agents may be administered simultaneously; in some embodiments, such agents may be administered sequentially; in some embodiments, such agents are administered in overlapping dosing regimens.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Effector Function: As used herein, “effector function” refers a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP) and complement-mediated cytotoxicity (CMC). In some embodiments, an effector function is one that operates after the binding of an antigen, one that operates independent of antigen binding, or both.

Effector Cell: As used herein, “effector cell” refers to a cell of the immune system that expresses one or more Fc receptors and mediates one or more effector functions. In some embodiments, effector cells may include, but may not be limited to, one or more of monocytes, macrophages, neutrophils, dendritic cells, eosinophils, mast cells, platelets, large granular lymphocytes, Langerhans’ cells, natural killer (NK) cells, T-lymphocytes, B-lymphocytes and may be from any organism including but not limited to humans, mice, rats, rabbits, and monkeys.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5’ cap formation, and/or 3’ end formation); (3) translation of an RNA into a polypeptide or protein; and/or (4) post-translational modification of a polypeptide or protein.

Fusion protein: As used herein, the term “fusion protein” generally refers to a polypeptide including at least two segments, each of which shows a high degree of amino acid identity to a peptide moiety that (1) occurs in nature, and/or (2) represents a functional domain of a polypeptide. Typically, a polypeptide containing at least two such segments is considered to be a fusion protein if the two segments are moieties that (1) are not included in nature in the same peptide, and/or (2) have not previously been linked to one another in a single polypeptide, and/or (3) have been linked to one another through action of the hand of man.

Gene: As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs, RNAi-inducing agents, etc. For the purpose of clarity we note that, as used in the present application, the term “gene” generally refers to a portion of a nucleic acid that encodes a protein; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a protein-coding nucleic acid.

Gene product or expression product: As used herein, the term “gene product” or “expression product” generally refers to an RNA transcribed from the gene (pre-and/or post-processing) or a polypeptide (pre- and/or post-modification) encoded by an RNA transcribed from the gene.

Immune response: As used herein, the term “immune response” refers to a response elicited in an animal. An immune response may refer to cellular immunity, humoral immunity or may involve both. An immune response may also be limited to a part of the immune system. For example, in certain embodiments, an immunogenic composition may induce an increased interferon gamma (IFN_{γ}) response. In certain embodiments, an immunogenic composition may induce a mucosal IgA response (e.g., as measured in nasal and/or rectal washes). In certain embodiments, an immunogenic composition may induce a systemic IgG response (e.g., as measured in serum). In certain embodiments, an immunogenic composition may induce virus-neutralizing antibodies or a neutralizing antibody response. In certain embodiments, an immunogenic composition may induce a cytolytic (CTL) response by T cells.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein.

Nucleic acid: As used herein, “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3 -methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5 -propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments, a nucleic acid has a nucleotide sequence comprising at least one element that encodes, or is the complement of a sequence that encodes, a polypeptide. In some embodiments, a nucleic acid has enzymatic activity.

Operably linked: As used herein, “operably linked” refers to a juxtaposition wherein the components described are in a relationship permitting them to function in their intended manner. A control sequence “operably linked” to one or more coding sequence(s) is ligated in such a way that expression of the one or more coding sequence(s) is achieved under conditions compatible with the control sequences. “Operably linked” sequences include both expression control sequences that are contiguous with the gene(s) of interest and expression control sequences that act in trans or at a distance to control the gene(s) of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site, and transcription termination sequence, while in eukaryotes, typically, such control sequences include promoters and transcription termination sequence. The term “control sequences” is intended to include components whose presence is essential for expression and processing, and can also include additional components whose presence is advantageous, for example, leader sequences and fusion partner sequences.

Paratope: As used herein, the term “paratope” refers to a portion of an antigen-binding polypeptide (e.g., antibody) that binds to an epitope of an antigen. As used herein, the term “biparatopic” (in the context of an antibody or a construct described herein) refers to an antibody or construct that includes two paratopes, each of which binds to a different epitope on a single antigen. As used herein, the term “multiparatopic” (in the context of an antibody or a construct described herein) refers to an antibody or construct that includes two or more paratopes, each of which binds to a different epitope on a single antigen. In some embodiments, the two or more paratopes of a multiparatopic antibody or a construct described herein bind to non-overlapping epitopes on a single antigen. In some embodiments, the two or more paratopes of a multiparatopic antibody or a construct described herein bind to two epitopes on a single antigen that can share 1, 2, or 3 amino acids.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. In some embodiments, a polypeptide may be longer than 5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Protein: As used herein, the term “protein”, refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

Reference: As used herein, “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, in the context of a subject (a patient or experimental organism), “response”, “responsive”, or “responsiveness” refers to an alteration in a subject’s condition that occurs as a result of, or correlates with, treatment. In certain embodiments, a response is a beneficial response. In certain embodiments, a beneficial response can include stabilization of a subject’s condition (e.g., prevention or delay of deterioration expected or typically observed to occur absent the treatment), amelioration (e.g., reduction in frequency and/or intensity) of one or more symptoms of the condition, and/or improvement in the prospects for cure of the condition, etc. In certain embodiments, for a subject who has cancer, a beneficial response can include: the subject has a positive clinical response to cancer therapy or a combination of therapies; the subject has a spontaneous response to a cancer; the subject is in partial or complete remission from cancer; the subject has cleared a cancer; the subject has not had a relapse, recurrence or metastasis of a cancer; the subject has a positive cancer prognosis; the subject has not experienced toxic responses or side effects to a cancer therapy or combination of therapies. In certain embodiments, for a subject who had cancer, the beneficial responses occurred in the past, or are ongoing.

In certain embodiments, a response is a non-beneficial response. In certain embodiments, a non-beneficial response can include deterioration of a subject’s condition, lack of amelioration (e.g., no reduction in frequency and/or intensity) of one or more symptoms of the condition, and/or degradation in the prospects for cure of the condition, etc. In certain embodiments, for a subject who has cancer, a non-beneficial response can include: the subject has a negative clinical response to cancer therapy or a combination of therapies; the subject is not in remission from cancer; the subject has not cleared a cancer; the subject has had a relapse, recurrence or metastasis of a cancer; the subject has a negative cancer prognosis; the subject has experienced toxic responses or side effects to a cancer therapy or combination of therapies. In certain embodiments, for a subject who had cancer, the non-beneficial responses occurred in the past, or are ongoing. In certain embodiments, presence, extent, and/or nature of response can be measured and/or characterized according to particular criteria. In certain embodiments, such criteria can include clinical criteria and/or objective criteria. In certain embodiments, techniques for assessing response can include, but are not limited to, clinical examination, positron emission tomography, chest X-ray, CT scan, MRI, ultrasound, endoscopy, laparoscopy, presence or level of a particular marker in a sample, cytology, and/or histology. Where a response of interest is a response of a tumor to a therapy, ones skilled in the art will be aware of a variety of established techniques for assessing such response, including, for example, for determining tumor burden, tumor size, tumor stage, etc. Methods and guidelines for assessing response to treatment are discussed in, e.g., Therasse et al., J. Natl. Cancer Inst., 2000, 92(3):205-216; and Seymour et al., Lancet Oncol., 2017, 18:e143-52. The exact response criteria can be selected in any appropriate manner, provided that when comparing groups of tumors, patients or experimental organism, and/or cells, organs, tissues, or cell components, the groups to be compared are assessed based on the same or comparable criteria for determining response rate. One of ordinary skill in the art will be able to select appropriate criteria.

Subject: By “subject” is meant a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Suffering from: An individual who is “suffering from” a disease, disorder, or condition (e.g., cancer) has been diagnosed with and/or exhibits one or more symptoms of the disease, disorder, or condition.

Symptoms are reduced: According to the present invention, “symptoms are reduced” when one or more symptoms of a particular disease, disorder or condition is reduced in magnitude (e.g., intensity, severity, etc.) or frequency. For purposes of clarity, a delay in the onset of a particular symptom is considered one form of reducing the frequency of that symptom. It is not intended that the present invention be limited only to cases where the symptoms are eliminated. The present invention specifically contemplates treatment such that one or more symptoms is/are reduced (and the condition of the subject is thereby “improved”), albeit not completely eliminated.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, “therapeutically effective amount” refers to an amount which, when administered to an individual in need thereof in the context of inventive therapy, will block, stabilize, attenuate, or reverse a cancer-supportive process occurring in said individual, or will enhance or increase a cancer-suppressive process in said individual. In the context of cancer treatment, a “therapeutically effective amount” is an amount which, when administered to an individual diagnosed with a cancer, will prevent, stabilize, inhibit, or reduce the further development of cancer in the individual. A particularly preferred “therapeutically effective amount” of a composition described herein reverses (in a therapeutic treatment) the development of a malignancy such as a pancreatic carcinoma or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor such as a pancreatic adenocarcinoma (e.g., over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this invention would be indicated by finding a decrease in one or more pro-angiogenic markers, an increase in anti-angiogenic markers, the normalization (i.e., alteration toward a state found in normal individuals not suffering from cancer) of metabolic pathways, intercellular signaling pathways, or intracellular signaling pathways that exhibit abnormal activity in individuals diagnosed with cancer. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Treatment: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Tumor infiltrating lymphocyte: As used herein, the term “tumor-infiltrating lymphocytes” refers to white blood cells of a subject afflicted with a cancer (such as melanoma), that have left the blood stream and have migrated into a tumor. In some embodiments, tumor-infiltrating lymphocytes have tumor specificity.

Vector: As used herein, “vector” refers to a nucleic acid molecule capable of transporting another nucleic acid to which it is associated. In some embodiments, vectors are capable of extra-chromosomal replication and/or expression of nucleic acids to which they are linked in a host cell such as a eukaryotic and/or prokaryotic cell. Vectors capable of directing the expression of operatively linked genes are referred to herein as “expression vectors.”

### DETAILED DESCRIPTION

Among other things, the present invention provides methods and compositions useful for treatment of cancer. Specifically, the present disclosure includes methods of treatment and compositions for the treatment of cancer relapse during or after therapy with a cellular therapeutic. In some embodiments, therapy with a cellular therapeutic is adoptive cell therapy (ACT), such as CAR-T cell therapy.

### Adoptive Cell Therapy Relapse

Adoptive cell therapy (ACT) is a potential therapeutic procedure in which cells are removed from a donor, cultured and/or manipulated ex vivo, and then administered to a patient for the treatment of a disease. A variety of cell types have been used in ACT in an attempt to treat several classes of disorders. In some embodiments, ACT comprises use of allogenic cells. In some embodiments, ACT comprises use of autologous cells. In some embodiments, ACT comprises use of a CAR-T cell, a CAR-NK cell, a TCR-T cell, a TIL cell, a allogenic NK cell, or a autologous NK cell. Generally, ACT comprises administration of a lymphocyte expressing an antigen receptor that binds a target antigen. In some embodiments, a target antigen is a tumor associated antigen (TAA) or tumor specific antigen (TSA) as described herein.

In some embodiments, the present disclosure provides methods of treatment and compositions for the treatment of cancer in subjects who previously responded to ACT (e.g., exhibited one or more clinically beneficial responses to ACT), and who no longer respond to ACT (e.g., exhibit a reduced level of one or more prior clinically beneficial responses to ACT and/or exhibit at least one nonbeneficial response to ACT). In some embodiments, the present disclosure provides methods of treatment and compositions for the treatment of multiple myeloma in subjects who previously responded to ACT and who are no longer responding to ACT. In some embodiments, the present disclosure provides compositions and methods comprising fusion proteins for the treatment of cancer relapse during or after ACT.

In some embodiments, a subject has received or is receiving ACT, previously exhibited at least one beneficial response to the ACT, and subsequently exhibited at least one nonbeneficial response to the ACT. In some embodiments, a subject has received or is receiving ACT and is treated by combination therapy with compositions and methods comprising fusion proteins for the treatment of cancer, and a response is measured subsequently. Whether a response is beneficial or nonbeneficial can be measured and/or characterized according to particular criteria. In certain embodiments, such criteria can include clinical criteria and/or objective criteria. In certain embodiments, techniques for assessing response can include, but are not limited to, clinical examination, positron emission tomography, chest X-ray, CT scan, MRI, ultrasound, endoscopy, laparoscopy, presence or level of a particular marker in a sample, cytology, and/or histology. A beneficial or nonbeneficial response of a tumor can be assessed by ones skilled in the art using a variety of established techniques for assessing such response, including, for example, for determining one or more of tumor burden, tumor size, tumor stage, etc. Methods and guidelines for assessing response to treatment are discussed in Therasse et al., J. Natl. Cancer Inst., 2000, 92(3):205-216; and Seymour et al., Lancet Oncol., 2017, 18:e143-52.

In some embodiments, a beneficial response results in a measured decrease in tumor burden, tumor size, and/or tumor stage (e.g., relative to tumor burden, tumor size, and/or tumor stage for the subject prior to initiation of ACT and/or for the subject at any stage during ACT). In some embodiments, a beneficial response is stability of disease (SD). In some embodiments, a beneficial response comprises clearance, regression, and/or or stabilization of the cancer, e.g., over a defined period of time (e.g., at least 1, 2, 3, 4, 5, 6, 7, 8, 9, 10, 11, 12 weeks, or at least 1, 2, 3, 4, 5, 6, 7, 8, 9, 10, 11, 12 months, or at least 1, 2, 3, 4, 5, 6, 7, 8, 9, 10, 11, 12 years). In some embodiments, a beneficial response comprises an absence of relapse, recurrence, and/or metastasis of the cancer, e.g., over a defined period of time (e.g., at least 1, 2, 3, 4, 5, 6, 7, 8, 9, 10, 11, 12 weeks, or at least 1, 2, 3, 4, 5, 6, 7, 8, 9, 10, 11, 12 months, or at least 1, 2, 3, 4, 5, 6, 7, 8, 9, 10, 11, 12 years).

In some embodiments, a nonbeneficial response results in a measured increase in tumor burden, tumor size, and/or tumor stage (e.g., relative to tumor burden, tumor size, and/or tumor stage for the subject following initiation of ACT and/or for the subject at any stage during ACT). In some embodiments, a subject exhibiting a nonbeneficial response to an ACT exhibits one or more sign or symptom of progressive disease (PD). In some embodiments, PD in multiple myeloma is defined as an increase of ≥ 25% in serum M-component with an absolute increase ≥ 0.5 g/dL; and/or urine M-component with an absolute increase ≥ 200 mg/24 hours; and/or the difference between involved and uninvolved free light chain levels with an absolute increase > 10 mg/dL; compared to a control sample or a previous sample from the subject (e.g., prior to initiation of ACT and/or smallest obtained value after initiation of ACT). In some embodiments, PD is defined as bone marrow plasma cell absolute percentage ≥ 10%; and/or development of new bone lesions or soft tissue plasmacytomas, or definite increase in the size of any existing bone lesions or soft tissue plasmacytomas (≥ 50% increase from nadir in size of >1 lesion, or a ≥ 50% increase in the longest diameter of a previous lesion >1 cm in short axis); and/or development of hypercalcemia (corrected serum calcium > 11.5 mg/dL or 2.65 mmol). In some embodiments, PD requires two consecutive assessments by the same method made at any time before classification as disease progression, and/or the start of any new therapy.

In some embodiments, relapse in multiple myeloma is defined as progressive disease. In some embodiments, relapse in multiple myeloma is defined as reappearance of serum or urine M-protein by immunofixation or electrophoresis; and/or development of ≥ 5% plasma cells in the bone marrow; and/or appearance of any other sign of progression (e.g., new plasmacytoma, lytic bone lesion, hypercalcemia).

In some embodiments, a subject that exhibits a nonbeneficial response demonstrates loss or downregulation of the target antigen of a cell used in ACT (e.g., relative to level of the target antigen prior to initiation of ACT and/or at any stage during ACT). In some embodiments, a tumor escapes ACT by exhibiting a lower antigen expression or demonstrating antigen loss (e.g., relative to level of antigen expression prior to initiation of ACT and/or at any stage during ACT). In some embodiments, a subject’s tumor escapes ACT by exhibiting lower antigen density (e.g., relative to level of antigen density prior to initiation of ACT and/or at any stage during ACT). In some embodiments, the antigen density on a subject’s tumor may be below a threshold needed for CAR-T activity (see e.g., Watanabe, K. et al. J. Immunol. 194, 911-920 (2015); Walker, A. J. et al. Mol. Ther. 25, 2189-2201 (2017)). In some embodiments, antigen expression is measured and/or compared relative to an appropriate control. In some embodiments, an appropriate control is the expression level and/or density of the ACT target antigen in the subject prior to the initiation of therapy with the ACT. In some embodiments, an appropriate control is the expression level and/or density of the ACT target antigen in an individual without cancer. In some embodiments, an appropriate control is the expression level and/or density of the ACT target antigen in a population.

Methods of the disclosure can be used to treat a subject who has been treated or is currently being treated with any ACT. Methods of the disclosure can be used to treat a subject who has been treated or is currently being treated with any CAR-T therapy. In some embodiments, methods of the disclosure can be used to treat a subject suffering multiple myeloma who has been treated or is currently being treated with any ACT. Exemplary ACT for the treatment of multiple myeloma include: Shah et al., Journal of Clinical Oncology 36, no. 15_suppl (May 20, 2018) 8006-8006; Kloess et al., Transfus Med Hemother 2019; 46:4-13; and Themeli et al., Nat Biotechnol, 31 (10), 928-33 Oct. 2013. In some embodiments, methods of the disclosure can be used to treat a subject suffering multiple myeloma who has been treated or is currently being treated with any CAR-T therapy. Exemplary CAR-T therapies for the treatment of multiple myeloma include: Brudno, J.N.et al., J. Clin. Oncol. 2018, 36, 2267-2280.; Cohen, A.D. et al.; J. Clin. Investig. 2019, 130; Raje, N et al. N. Engl. J. Med. 2019, 380, 1726-1737; Xu, J.; et al. Proc. Natl. Acad. Sci. USA 2019, 116, 9543-9551; Mailankody, S. et al.; Blood 2018, 132 (Suppl. 1), 959; Li, C.et al.;. Blood 2018, 132 (Suppl. 1), 1013 Wang, BY et al., Blood (2019) 134 (Supplement_1): 579; Raje et al., N Engl J Med 2019; 380:1726-1737.

In some embodiments, a fusion protein of the present disclosure is administered to a subject prior to receiving ACT. In some embodiments, a fusion protein of the present disclosure is administered to a subject concurrently with ACT. In some embodiments, a fusion protein of the present disclosure is administered to a subject after the subject has received or is receiving ACT.

In some embodiments,

### CAR-T Therapy Relapse

Generally, CAR-T therapy comprises administration of a T-cell expressing a chimeric antigen receptor (CAR) that binds a target antigen. In some embodiments, a CAR-T target antigen is a tumor associated antigen (TAA) or tumor specific antigen (TSA) as described herein.

In some embodiments, the present disclosure is based, in part, on the recognition that certain individuals being treated for cancer with CAR-T therapy who relapse (e.g., cease to exhibit one or more beneficial responses to CAR-T therapy, as described herein) can be “rescued” from relapse by administration of a fusion protein described herein. In some embodiments, the present disclosure provides compositions and methods comprising fusion proteins for the treatment of a subject exhibiting cancer relapse during or after CAR-T therapy.

In some embodiments, the present disclosure is based, in part, on the recognition that certain individuals being treated for cancer with CAR-T therapy will have a suboptimal response to therapy, and therefore may relapse, and therefore are treated to prevent relapse. In some embodiments, the present disclosure provides compositions and methods comprising fusion proteins for the treatment of a subject expected to have, or having, a sub-optimal responses to CAR T cell therapy, for example, patients who have achieved stable disease, partial response, very good partial response or complete response without achieving minimal residual disease-negative status. (see, e.g., www.cibmtr.org/manuals/fim/1/en/topic/multiple-myeloma-response-criteria).

Positive results have been observed with CAR-T therapy for treatment of multiple myeloma (Hosen, Cancers 2019, 11, 2024). However, due to relapse after CAR-T therapy for treatment of multiple myeloma the long-term efficacy of BCMA CAR T-cell is unsatisfactory, and curing MM is still difficult. See for example, Wang, BY et al., Blood (2019) 134 (Supplement 1): 579; Raje et al., N Engl J Med 2019; 380:1726-1737.

### Fusion Proteins

The present disclosure provides, among other things, fusion proteins. In some embodiments, a fusion protein described herein comprises one or more antigen-binding polypeptide(s) (or an antigen-binding fragment thereof) and one or more polypeptide antigen(s). In some embodiments, a fusion protein is a “binding protein” or “bridging protein”, e.g., that binds or bridges a tumor antigen described herein and a cell, e.g., a cell administered as part of ACT (e.g., a CAR-T cell) described herein. In the broadest sense, a fusion protein described herein comprises (i) one or more antigen-binding polypeptides that bind a tumor antigen; and (ii) one or more polypeptide antigens that is the target of an ACT (e.g., a CAR-T cell), and the fusion protein “bridges” such ACT (e.g., CAR-T cell) with such tumor antigen. For example, in some embodiments, a fusion protein described herein comprises (i) one or more antigen-binding polypeptides that bind a first multiple myeloma antigen; and (ii) a second multiple myeloma antigen (e.g., different from the first multiple myeloma antigen) that is the target of ACT (e.g., a CAR-T cell), and the fusion protein “bridges” such ACT (e.g., CAR-T cell) with such first multiple myeloma antigen.

In some embodiments, the one or more polypeptide antigen(s) is linked (e.g., fused) to the amino terminus of one of the one or more antigen binding polypeptides. In some embodiments, the one or more polypeptide antigen(s) is linked (e.g., fused) to the carboxy terminus of one of the one or more antigen binding polypeptides.

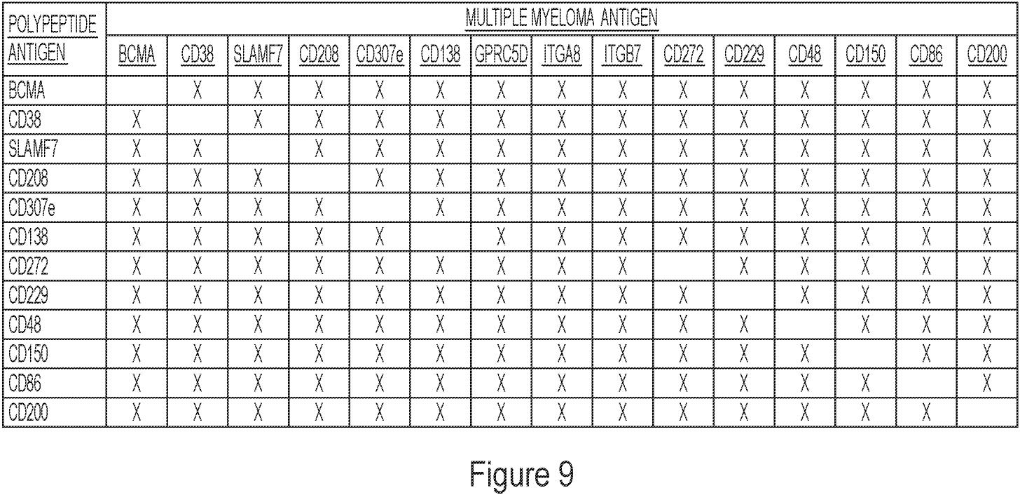

In some embodiments, a fusion protein described herein comprises 2, 3, 4, or more antigen-binding polypeptides (or an antigen-binding fragment thereof) and a polypeptide antigen. For example, in some embodiments, a fusion protein described herein comprises (i) an antigen-binding polypeptide (or an antigen-binding fragment thereof) that binds to CD38, (ii) an antigen-binding polypeptide (or an antigen-binding fragment thereof) that binds to GPRC5D, and (iii) a BCMA polypeptide. Other fusion proteins of the disclosure include, e.g., 2, 3 or more different antigen binding polypeptides, each of which binds to a different multiple myeloma antigen described herein (e.g., shown in FIG. 9), and (ii) a polypeptide antigen described herein (e.g., shown in FIG. 9).

### Half-Life Extending Moiety

In some embodiments, a fusion protein described herein comprises at least one heterologous moiety that is a “half-life extending moiety”. Half-life extending moieties can comprise, for example, (i) XTEN polypeptides; (ii) Fc; (iii) human serum albumin (HSA), (iv) albumin binding polypeptide or fatty acid, (v) the C-terminal peptide (CTP) of the beta subunit of human chorionic gonadotropin, (vi)proline-alanine-serine polymer (PAS); (vii) homo-amino acid polymer(HAP); (viii) human transferrin; (ix) polyethylene glycol (PEG); (x)hydroxyethyl starch (HES), (xi) polysialic acids (PSAs); (xii) a clearance receptor or fragment thereof which blocks binding of the chimeric molecule to a clearance receptor; (xiii) low complexitypeptides; (xiv) vWF; (xv) elastin-like peptide (ELP) repeat sequence; (xvi) fusion with artificial GLK; or (xv) any combinations thereof. See, e.g., Strohl, BioDrugs, 29:215-239 (2015).

In some embodiments, the half-life extending moiety comprises or consists of an XTEN polypeptide. Non-limiting, examples of XTENs are disclosed in U.S. Pat. Publication No. 2012/0263701 and WO 2016/065301.

In some embodiments, the half-life extending moiety comprises an Fc region, e.g., the hinge, CH2 and CH3 domains, e.g., from IgG1, IgG2, or IgG4. The Fc region may include one or more substitutions that reduce effector function. For example, the Fc region is from IgG2 and may comprise one or both of these mutations: V234A andG237A, that can reduce effector function. Exemplary heterologous moieties also include, e.g., FcRn binding moieties (e.g., complete Fc regions or portions thereof which bind to FcRn), single chain Fc regions (scFc regions, e.g., as described in U.S. Publ.No. 2008/0260738, and Intl. Publ. Nos. WO 2008/012543 and WO2008/1439545), or processable scFc regions. In some embodiments, a heterologous moiety can include an attachment site for a non-polypeptide moiety such as polyethylene glycol (PEG), hydroxyethyl starch (HES), polysialic acid, or any derivatives, variants, or combinations of these moieties.

In some embodiments, the half-life extending moiety comprises human serum albumin (HSA) or a functional fragment thereof. Examples of albumin or the fragments or variants thereof are disclosed in, e.g., US Pat. Publ. Nos. US2008/0194481, US2008/0004206, US2008/0161243, US2008/0261877, or US2008/0153751 or PCT Appl. Publ. Nos. WO2008/033413, WO2009/058322, or WO2007/021494. In certain instances, the half-life extending moiety can comprise an albumin binding moiety, which comprises an albumin binding peptide, a bacterial albumin binding domain, an albumin-binding antibody fragment, or any combinations thereof. For example, the albumin binding protein can be a bacterial albumin binding protein, an antibody or an antibody fragment including domain antibodies (see, e.g., U.S. Pat. No. 6,696,245). An albumin binding protein, for example, can be a bacterial albumin binding domain, such as the one of streptococcal protein G (Konigand Skerra (1998) J. Immunol. Methods 218, 73-83). Other examples of albumin binding peptides that can be used are described in, e.g., U.S. Pub. No. US2003/0069395; U.S. Pub. No. US2007/0269422; Vosjan M et al., Mol Cancer Ther; 11(4): 1017-25 or Dennis etal. (2002) J. Biol. Chem. 277, 35035-35043.

In certain embodiments, the half-life extending moiety can comprise a beta subunit of the C-terminal peptide (CTP) of human chorionic gonadotropin or fragment, variant, or derivative thereof. The insertion of one or more CTP peptides into a recombinant protein is known to increase the in vivo half-life of that protein. See, e.g., U.S. Pat. No.5,712,122 and U.S. Patent Appl. Publ. No. US 2009/0087411.

In certain embodiments, the half-life extending moiety can comprise a PAS sequence. A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part a fusion protein described herein. Non-limiting examples of PAS sequences are disclosed in, e.g., US Pat. Publ. No. 2010/0292130 and PCT Appl. Publ. No.WO2008/155134 A1.

In some embodiments, the half-life extending moiety is a soluble polymer including, but not limited to, polyethylene glycol (PEG), ethylene glycol/propylene glycol copolymers, carboxymethylcellulose, dextran, or polyvinyl alcohol. In one embodiment, the half-life extending moiety is PEG. The polyethylene glycol can have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000,60,000, 65,000, 70,000, 75,000, 80,000, 85,000, 90,000, 95,000, or 100,000 kDa. In some embodiments, the polyethylene glycol can have a branched structure as described in, e.g., U.S. Pat. No. 5,643,575; Morpurgo et al., Appl. Biochem.Biotechnol. 56:59-72 (1996); Vorobjev et al., Nucleosides Nucleotides18:2745-2750 (1999); and Caliceti et al., Bioconjug. Chem. 10:638-646(1999).

### Antigen Binding Polypeptide

As described herein, the disclosure provides fusion proteins including one or more antigen binding polypeptide, or a fragment thereof. In some embodiments, an antigen binding polypeptide targets a tumor antigen, e.g., a tumor-specific antigen (TSA) or a tumor-associated antigen (TAA). A TSA is (or is believed to be) unique to tumor cells and does not occur on other cells in the body (e.g., does not occur to a significant extent on other cells). A TAA is not unique to a tumor cell and instead is also expressed on a normal cell (e.g., expressed under conditions that fail to induce a state of immunologic tolerance to the antigen). For example, TAAs can be antigens that are expressed on normal cells during fetal development when the immune system is immature and unable to respond, or they can be antigens that are normally present at low levels on normal cells but that are expressed at higher levels on tumor cells.

In some embodiments, an antigen binding polypeptide binds a tumor antigen that is or comprises one or more antigenic cancer epitopes associated with multiple myeloma. In some embodiments, an antigen binding polypeptide targets and/or binds to one or more of the following tumor antigens: CD38; CS1/SLAMF7; GPRC5D; CD208 (LAMP3); CD307e (FCRL5); ITGA8 (integrin a8); CD138; ITGB7 (activated integrin beta-7), CD272; CD229; CD48; CD150; CD86; CD200; BAFF-R (TNFRSF13C) and/or BCMA. In some embodiments, an antigen binding polypeptide targets and/or binds to one or more of the antigens identified in Frigyesi et al., Blood. 2014;123(9):1336-1340, Hosen et al. Nat Med. 2017 Dec;23(12):1436-1443, or Muccio et al., Cytometry Part B (Clinical Cytometry) 90B:81-90 (2016). Various review articles have been published that describe useful anti-tumor antibodies (see, for example, Adler et al., Hematol. Oncol. Clin. North Am. 26:447-81 (2012); Li et al., Drug Discov. Ther. 7:178-84 (2013); Scott et al., Cancer Immun. 12:14 (2012); and Sliwkowski et al., Science 341:1192-1198 (2013)). Exemplary antigen binding polypeptides include, e.g., daratumumab, felzartamab (MOR202) isatuximab; Elotuzumab, BT062, HuLuc63, belantamab mafodotin (GSK2857916), indatuximab ravtansin; azintuxizumab vedotin (ABBV-838).

In some embodiments, an antigen binding polypeptide comprises or consists of the amino acid sequence of SEQ ID NOs: 15-18; or 25, 27, 29, 31, 33, 35, 37, 39, 57 or 58. In some embodiments, an antigen binding polypeptide comprises or consists of an amino acid sequence having at least 80%, 85%, 90%, 91%, 92%, 93%, 94%, 95%, 96%, 97%, 98%, or 99% identity to SEQ ID NOs: 15-18; or 25, 27, 29, 31, 33, 35, 37, 39, 57 or 58.

In some embodiments, an antigen binding polypeptide targets and/or binds to one or more post translational modifications made to a protein on a tumor. In some embodiments, an antigen binding polypeptide targets and/or binds to one or more glycosyl modifications on a protein on a tumor. In some embodiments an antigen binding polypeptide targets and/or binds to Tn (GalNAcα1-O-Ser/Thr) and/or sialyl-Tn (STn) (NeuAcα2-6-GalNAcα1-O-Ser/Thr) glycoforms. See Posey et al., Immunity 44, 1444-1454, Jun. 21, 2016.

In some embodiments, an antigen binding polypeptide is an antibody or fragment thereof. In some embodiments, an antibody or fragment thereof includes, e.g., intact IgG, IgE, IgA or IgM, bi- or multi- specific antibodies (e.g., Zybodies®, etc), single chain Fvs, polypeptide-Fc fusions, Fabs, camelid antibodies, masked antibodies (e.g., Probodies®), Small Modular ImmunoPharmaceuticals (“SMIPsTM”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®.

In some embodiments, an antigen-binding polypeptide is a bispecific antibody or portion thereof. In some embodiments, such a bispecific antibody or portion thereof binds one or more tumor antigens described herein, e.g., that together define a specific tumor type.

In some embodiments, a fusion protein is a biparatopic fusion protein. In some embodiments, a biparatopic fusion protein comprises two or more antigen binding polypeptides and at least one polypeptide antigen described herein. In some embodiments, the two or more antigen binding polypeptides bind different epitopes of the same tumor antigen described herein. In some embodiments, a biparatopic fusion protein is or includes two antibody fragments and at least one additional non-antibody polypeptide. In some embodiments, a fusion protein is or includes an scFv, a VHH, and at least one polypeptide antigen.

The two or more antigen binding polypeptides and at least one polypeptide antigen can be configured in any order within a biparatopic fusion protein. In some embodiments, the polypeptide antigen is linked (e.g., fused) to the amino terminus of one of the two or more antigen binding polypeptides. In some embodiments, the polypeptide antigen is linked (e.g., fused) to the carboxyl terminus of one of the two or more antigen binding polypeptides. For example, a biparatopic fusion protein that includes antigen binding polypeptide A; antigen binding polypeptide B; and a polypeptide antigen can be configured in any of the following configurations: (i) antigen binding polypeptide A- antigen binding polypeptide B-polypeptide antigen; (ii) antigen binding polypeptide B- antigen binding polypeptide A-polypeptide antigen; (iii) polypeptide antigen-antigen binding polypeptide A- antigen binding polypeptide B; (iv) polypeptide antigen-antigen binding polypeptide B- antigen binding polypeptide A; (v) antigen binding polypeptide B- polypeptide antigen- antigen binding polypeptide A; (vi) antigen binding polypeptide A- polypeptide antigen- antigen binding polypeptide B.

In some embodiments, a fusion protein described herein comprises two or more antigen-binding polypeptide(s) (or an antigen-binding fragment thereof) and one or more polypeptide antigen(s) (e.g., a fusion protein described herein is bivalent). In some embodiments, two or more antigen binding polypeptides of a fusion protein described herein bind the same antigen. In some embodiments, two or more antigen binding polypeptides of a fusion protein described herein bind the same epitope. In some embodiments, the two or more antigen binding polypeptides are the same polypeptide.

In some embodiments, two or more antigen binding polypeptides are low affinity binders. In some embodiments, a fusion protein comprises two or more antigen binding polypeptides, wherein the two or more antigen binding polypeptides specifically bind to a target antigen with low affinity. In some embodiments, a low affinity antigen binding polypeptide binds to a target antigen with Kd of between about 50 nM to about 2 µM. In some embodiments, a low affinity antigen binding polypeptide binds to a target antigen with Kd of between about 50-100 nM; 75-125 nM; 100-150 nM; 125-175 nM; 150-200 nM; 175-225 nM; 200-250 nM; 225-275 nM; 250-300 nM; 275-325 nM; 300-350 nM; 325-375 nM; 350-400 nM; 375-425 nM; 400-450 nM; 425-475 nM; 450-500 nM; 475-525 nM; 500-550 nM; 525-575 nM; 550-600 nM; 575-625 nM; 600-650 nM; 625-675 nM; 650-700 nM; 675-725 nM; 700-750 nM; 725-775 nM;750-800 nM; 775-825 nM; 800-850 nM; 825-875 nM; 850-900 nM; 875-925 nM; 900-950 nM; 925-975 nM; 950-1.0 µM; 975-1.25 µM; 1.0-1.50 µM; 1.25-1.75 µM; 1.50-2.00 µM; 1.75-2.25 µM; 2.0-2.50 µM; 50-100 nM; 100-200 nM; 200-300 nM; 300-400 nM; 400-500 nM; 500-600 nM; 600-700 nM; 700-800 nM; 800-900 nM; 900 nM-1.0µM; 1.0 µM-1.1µM; 1.1 µM-1.2µM; 1.2 µM-1.3µM; 1.3 µM-1.4µM; 1.4 µM-1.5µM; 1.5 µM-1.6µM; 1.6 µM-1.7µM; 1.7 µM-1.8µM; 1.8 µM-1.9µM; 1.9 µM-2.0µM.

In some embodiments, a fusion protein described herein comprises two or more antigen binding polypeptides, each of which specifically binds to a target antigen with low affinity (e.g., as described herein), and such fusion protein binds with high avidity (e.g., as described herein) to a target cell (e.g., a cell expressing the target antigen). In some embodiments, a fusion protein comprising two or more antigen binding polypeptides, each of which specifically binds to a target antigen with low affinity, binds with high avidity (e.g., as described herein) to a target cell expressing the target antigen at a high level, e.g., a higher level relative to a control level (e.g., level of target antigen on a healthy cell or average level of target antigen on a population of healthy cells). In some embodiments, a fusion protein comprising two or more antigen binding polypeptides, each of which specifically binds to a target antigen with low affinity, binds with low avidity (e.g., as described herein) to a non-target cell (e.g., a healthy cell) expressing the target antigen at a low level, e.g., a lower level relative to a control level (e.g., level of target antigen on a target tumor cell or average level of target antigen on a population of target tumor cells). In some embodiments, such a fusion protein binds with high avidity to a target cell (e.g., with a Kd of about 0.00025, 0.0005, 0.00075, 0.001, 0.0025; 0.005, 0.0075, 0.01, 0. 025, 0.05, 0.075, 0.1, 0.25, 0.5, 0.75, 1, 2, 5, 10, 15, 20, 25, 30, 35, 40 nM). In some embodiments, such a fusion protein binds with low avidity to a non-target cell (e.g., with a Kd of greater than about 40 nM).

Methods of producing antigen binding polypeptides (e.g., antibodies or antigen binding fragments) that bind a target antigen with low affinity and methods of making constructs that selectively bind (e.g., with higher avidity) to one population of cells (e.g., a population of cells that expresses a high level of antigen) relative to a second population of cells (e.g., a population of cell that expresses a low level of antigen) are known in the art and have been described in, for example, Bacac et al., Clin Cancer Res; 22(13) Jul. 1, 2016; US20200216559A1; US2020/0199251; US 2013/0209355; Drent et al., Molecular Therapy Vol. 25 No 8 Aug. 2017; and Seckinger et al., Cancer Cell 31, 396-410, Mar. 13, 2017; each of which is incorporated by reference herein. Methods of determining affinity of an antigen binding polypeptide are known in the art and described by references cited herein. In some embodiments, affinity of an antigen binding polypeptide can be measured by surface plasmon resonance (e.g., Biacore). In some embodiments, affinity of an antigen binding polypeptide can be measured by biolayer interferometry. In some embodiments, affinity of an antigen binding polypeptide is measured by binding to a cell expressing said antigen. In some embodiments, affinity of an antigen binding polypeptide is measured by fluorescence-activated cell sorting (FACS). In some embodiments, affinity of an antigen binding polypeptide is measured by enzyme-linked immunosorbent assay (ELISA).

In some embodiments, at least one of two or more antigen binding polypeptides in a fusion protein described herein binds CD38. In some embodiments, one or more of two or more antigen binding polypeptides in a fusion protein described herein is or comprises an antibody or antigen binding fragment thereof as described in Drent et al., Molecular Therapy Vol. 25 No 8 Aug. 2017; US 2013/0209355; or US 2020/0199251 each of which is incorporated by reference herein.

Antibodies or fragments can be produced by any method known in the art for synthesizing antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using methods described in, e.g., Morrison, 1985, Science 229:1202, and humanized antibodies by methods described in, e.g., U.S. Pat. No. 6,180,370.

### Polypeptide Antigens

As described herein, in some embodiments, fusion proteins include a polypeptide antigen. In some embodiments, a polypeptide antigen is a tumor antigen described herein.

In some embodiments, a polypeptide antigen is a target of (e.g., binds to or is recognized by) a cell that is delivered or administered to a subject as part of ACT. For example, in some embodiments, a polypeptide antigen is a target of (e.g., binds to or is recognized by) an antigen receptor on a cell administered in ACT, e.g., a CAR-bearing cell (e.g., a CAR-T cell). In some embodiments, a subject has received or is receiving therapy with a CAR-T cell, and the polypeptide antigen included in a fusion protein described herein is the same target antigen of the CAR-T cell. In some embodiments, a subject has received or is receiving therapy with a first CAR-T cell, and the polypeptide antigen included in a fusion protein described herein is a target antigen that is different from the target antigen of the first CAR-T cell, e.g., is the same as a target antigen of a second CAR-T cell.

In some embodiments, a fusion protein described herein includes a polypeptide antigen selected from the group consisting of BCMA, CD38, SLAMF7, CD208, CD307e, ITGA8; ITGB7; CD272, CD229, CD48, CD150, CD86, CD200; BAFF-R (TNFRSF13C) and CD138.

In some embodiments, a fusion protein described herein includes a combination of (i) an antigen binding polypeptide that binds to a multiple myeloma antigen and (ii) a polypeptide antigen as shown in FIG. 9.

### Protein Therapeutics

In some embodiments, fusion proteins as described herein can be produced and used as therapeutics. Such polypeptides can be included in a composition, e.g., a pharmaceutical composition, and used as a protein therapeutic.

A variety of methods of making polypeptides are known in the art and can be used to make a polypeptide to be included in a protein therapeutic. For example, a polypeptide can be recombinantly produced by utilizing a host cell system engineered to express a nucleic acid encoding the polypeptide. Recombinant expression of a gene can include construction of an expression vector containing a polynucleotide that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and transfected cells can then be cultured by conventional techniques to produce polypeptide.

A variety of host expression vector systems can be used (see, e.g., U.S. Pat. No. 5,807,715). Such host-expression systems can be used to produce polypeptides and, where desired, subsequently purified. Such host expression systems include microorganisms such as bacteria (e.g., E.coli and B.subtilis) transformed with recombinant bacteriophage DNA, plasmid DNA or cosmid DNA expression vectors containing polypeptide coding sequences; yeast (e.g., Saccharomyces and Pichia) transformed with recombinant yeast expression vectors containing polypeptide coding sequences; insect cell systems infected with recombinant virus expression vectors (e.g., baculovirus) containing polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing polypeptide coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5 K promoter).

For bacterial systems, a number of expression vectors can be used, including, but not limited to, the E.coli expression vector pUR278 (Ruther et al., 1983, EMBO 12:1791); pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST).

For expression in mammalian host cells, viral-based expression systems can be utilized (see, e.g., Logan & Shenk, 1984, Proc. Natl. Acad. Sci. USA 8 1:355-359). The efficiency of expression can be enhanced by inclusion of appropriate transcription enhancer elements, transcription terminators, etc. (see, e.g., Bittner et al., 1987, Methods in Enzymol. 153:516-544).

In addition, a host cell strain can be chosen that modulates expression of inserted sequences, or modifies and processes the gene product in the specific fashion desired. Different host cells have characteristic and specific mechanisms for post-translational processing and modification of proteins and gene products. Appropriate cell lines or host systems can be chosen to ensure the correct modification and processing of the polypeptide expressed. Such cells include, for example, established mammalian cell lines and insect cell lines, animal cells, fungal cells, and yeast cells. Mammalian host cells include, e.g., BALB/c mouse myeloma line (NSO/1, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., J. Gen Virol., 36:59,1977); human fibrosarcoma cell line (e.g., HT1080); baby hamster kidney cells (BHK, ATCC CCL 10); Chinese hamster ovary cells +/-DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

For long-term, high-yield production of recombinant proteins, host cells are engineered to stably express a polypeptide. Host cells can be transformed with DNA controlled by appropriate expression control elements known in the art, including promoter, enhancer, sequences, transcription terminators, polyadenylation sites, and selectable markers. Methods commonly known in the art of recombinant DNA technology can be used to select a desired recombinant clone.

Once a protein described herein has been produced by recombinant expression, it may be purified by any method known in the art for purification, for example, by chromatography (e.g., ion exchange, affinity, and sizing column chromatography), centrifugation, differential solubility, or by any other standard technique for purification of proteins. For example, an antibody can be isolated and purified by appropriately selecting and combining affinity columns such as Protein A column with chromatography columns, filtration, ultra filtration, salting-out and dialysis procedures (see Antibodies: A Laboratory Manual, Ed Harlow, David Lane, Cold Spring Harbor Laboratory, 1988). Further, as described herein, a polypeptide can be fused to heterologous polypeptide sequences to facilitate purification. Alternatively or additionally, a polypeptide or fusion protein can be partially or fully prepared by chemical synthesis.

### Viral Delivery

In some embodiments, a nucleic acid encoding a fusion protein described herein can be introduced in a viral vector. In some embodiments, such a viral vector can be used to introduce a fusion protein into a cancer cell (e.g., a tumor cell). Introduction of such fusion protein can increase susceptibility to a subject’s immune system and/or one or more additional therapeutic agents (see, e.g., WO2017/075533).

### Vector Design

A nucleic acid sequence encoding a fusion protein described herein can be cloned into a number of types of vectors. For example, a nucleic acid can be cloned into a plasmid, a phagemid, a phage derivative, an animal virus, and a cosmid. Other vectors can include expression vectors, replication vectors, probe generation vectors, sequencing vectors, and viral vectors. In other examples, the vector can be a foamy viral (FV) vector, a type of retroviral vector made from spumavirus. Viral vector design and technology is well known in the art as described in Sambrook et al, (Molecular Cloning: A Laboratory Manual, 2001), and in other virology and molecular biology manuals.

### Viral Transduction

Viruses are highly efficient at nucleic acid delivery to specific cell types, while often avoiding detection by the infected host immune system. These features make certain viruses attractive candidates as vehicles for introduction of cellular therapy targets into cancer cells, e.g., solid tumor cells. A number of viral based systems have been developed for gene transfer into mammalian cells. Examples of viral vectors include, but are not limited to, retroviruses, adenoviruses, adeno-associated viruses, herpes viruses, lentiviruses, poxviruses, herpes simplex 1 virus, herpes virus, oncoviruses (e.g., murine leukemia viruses), and the like. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Lentiviral and Retroviral transduction can be enhanced by the addition of polybrene (SantaCruz sc-134220; Millipore TR-1003-G; Sigma 107689), a cationic polymer (also known as hexamehtrine bromide) that is used to increase the efficiency of the retrovirus transduction.

For example, retroviruses provide a platform for gene delivery systems. Retroviruses are enveloped viruses that belong to the viral family Retroviridae. Once in a host’s cell, the virus replicates by using a viral reverse transcriptase enzyme to transcribe its RNA into DNA. The retroviral DNA replicates as part of the host genome, and is referred to as a provirus. A selected gene can be inserted into a vector and packaged in retroviral particles using techniques known in the art. The recombinant virus can then be isolated and delivered to cells of the subject in vivo. A number of retroviral systems are known in the art, (see, e.g., U.S. Pat Nos.5,994,136, 6,165, 782, and 6,428,953).

Retroviruses include the genus of Alpharetrovirus (e.g., avian leukosis virus), the genus of Betaretrovirus; (e.g., mouse mammary tumor virus) the genus of Deltaretrovirus (e.g., bovine leukemia virus and human T-lymphotropic virus), the genus of Epsilonretrovirus (e.g., Walleye dermal sarcoma virus), and the genus of Lentivirus. In some embodiments, a retrovirus is a lentivirus a genus of viruses of the Retroviridae family, e.g., characterized by a long incubation period. Lentiviruses are unique among the retroviruses in being able to infect non-dividing cells; they can deliver a significant amount of genetic information into the DNA of the host cell, so can be used as an efficient gene delivery vector. In some examples, a lentivirus can be, but not limited to, human immunodeficiency viruses (HIV-1 and HIV-2), simian immunodeficiency virus (S1V), feline immunodeficiency virus (FIV), equine infections anemia (EIA), and visna virus. Vectors derived from lentiviruses offer the means to achieve significant levels of gene transfer in vivo.

In some embodiments, a vector is an adenovirus vector. Adenoviruses are a large family of viruses containing double stranded DNA. They replicate the DNA of the host cell, while using a host’s cell machinery to synthesize viral RNA DNA and proteins. Adenoviruses are known in the art to affect both replicating and non-replicating cells, to accommodate large transgenes, and to code for proteins without integrating into the host cell genome.

In some embodiments, an AAVP vector is used. An AAVP vector is a hybrid of prokaryotic-eukaryotic vectors, which are chimeras of genetic cis-elements of recombinant adeno-associated virus and phage. An AAVP combines selected elements of both phage and AAV vector systems, providing a vector that is simple to produce in bacteria and can exhibit little or no packaging limit, while allowing infection of mammalian cells combined with integration into the host chromosome. Vectors containing many of the appropriate elements are commercially available, and can be further modified by standard methodologies to include the necessary sequences. Among other things, AAVPs do not require helper viruses or trans-acting factors. In addition, the native tropism of AAV for mammalian cells is eliminated since there is not AAV capsid formation. Other methods and details are in U.S. Pat. 8,470,528 and Hajitou A. et al., Cell, 125: 358-398.

In some embodiments, a human papilloma (HPV) pseudovirus is used. DNA plasmids can be packaged into papillomavirus L1 and L2 capsid protein to generate pseudovirion that can efficiently deliver DNA. The encapsulation can protect the DNA from nucleases and provides a targeted delivery with a high level of stability. Many of the safety concerns associated with the use of viral vectors can be mitigated with an HPV pseudovirus. Other methods and examples are in Hung, C., et al.,Plos One, 7:7(e40983); 2012, U.S. Pat. 8,394,411, and Kines, R., et al Int J of Cancer, 2015.

In some embodiments, an oncolytic virus is used. Oncolytic virus therapy can selectively replicate the virus in cancer cells, and can subsequently spread within a tumor, e.g., without affecting normal tissue. Alternatively, an oncolytic virus can preferentially infect and kill cells without causing damage to normal tissues. Oncolytic viruses can also effectively induce immune responses to themselves as well as to the infected tumor cell. Typically, oncolytic viruses fall into two classes: (I) viruses that naturally replicate preferentially in cancer cells and are nonpathogenic in humans. Exemplary class (I) oncolytic viruses include autonomous parvoviruses, myxoma virus (poxvirus), Newcastle disease virus (NDV; paramyxovirus), reovirus, and Seneca valley virus (picornavirus). A second class (II), includes viruses that are genetically manipulated for use as vaccine vectors, including measles virus (paramyxovirus), poliovirus (picornavirus), and vaccinia virus (poxvirus). Additionally, oncolytic viruses may include those genetically engineered with mutations/deletions in genes required for replication in normal but not in cancer cells including adenovirus, herpes simplex virus, and vesicular stomatitis virus. Oncolytic viruses can be used as a viral transduction method due to their low probability of genetic resistance because they can target multiple pathways and replicate in a tumor-selective method. The viral dose within a tumor can increase over time due to in situ viral amplification (as compared to small molecule therapies which decrease with time), and safety features can be built in (i.e., drug and immune sensitivity).

### Administration

Certain embodiments of the disclosure include methods of administering to a subject a protein therapeutic described herein and/or a composition comprising a protein therapeutic, e.g., in an amount effective to treat a subject. In some embodiments, the method effectively treats cancer in the subject.

A polypeptide (e.g., a protein therapeutic) described herein can be incorporated into a pharmaceutical composition (e.g., for use as a protein therapeutic). Pharmaceutical compositions comprising a polypeptide can be formulated by methods known to those skilled in the art (see, e.g., Remington’s Pharmaceutical Sciences pp. 1447-1676 (Alfonso R. Gennaro, ed., 19th ed. 1995)). Pharmaceutical composition can be administered parenterally in the form of an injectable formulation comprising a sterile solution or suspension in water or another pharmaceutically acceptable liquid. For example, a pharmaceutical composition can be formulated by suitably combining a polypeptide with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in pharmaceutical preparations is such that a suitable dose within the designated range is provided.

The sterile composition for injection can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection.

A suitable means of administration can be selected based on the age and condition of the subject. A single dose of a pharmaceutical composition containing a polypeptide can be selected from a range of 0.001 to 1000 mg/kg of body weight. On the other hand, a dose can be selected in the range of 0.001 to 100000 mg/body weight, but the present disclosure is not limited to such ranges. Dose and method of administration can vary depending on the weight, age, condition, and the like of the subject, and can be suitably selected as needed by those skilled in the art.

### Subject Identification

In some embodiments, a subject is identified and/or selected for administration of fusion protein as described herein. For example, in some embodiments, a subject can be identified and/or selected for treatment based on diagnosis of multiple myeloma. In some embodiments, a subject can be identified and/or selected for treatment based on diagnosis of refractory or resistant multiple myeloma. In some embodiments, a subject can be identified and/or selected for treatment based on the prescription to receive ACT therapy. In some embodiments, a subject can be identified and/or selected for treatment based on evidence of ACT therapy relapse. In some embodiments, a subject can be identified and/or selected for treatment based on one or more measured or observed sign of relapse in multiple myeloma (e.g., a non-beneficial response, loss or downregulation of the target antigen of a cell used in ACT or progressive disease). In some embodiments, the fusion protein is administered to the subject. In some embodiments, upon administration of the fusion protein therapy, the subject exhibits a positive clinical response to the ACT therapy, e.g., exhibits an improvement based on one or more clinical and/or objective criteria (e.g., exhibits a decrease in tumor burden, tumor size, and/or tumor stage).

Methods described herein can include preparing and/or providing a report, such as in electronic, web-based, or paper form. The report can include one or more outputs from a method described herein, e.g., tumor burden, tumor size, and/or tumor stage, stability of disease, loss or downregulation of target antigen. In some embodiments, a report is generated, such as in paper or electronic form, which identifies the presence or absence of one or more tumor antigens for a cancer patient, and optionally, a recommended course of cancer therapy. In some embodiments, the report includes an identifier for the cancer patient. In one embodiment, the report is in web-based form.

In some embodiments, additionally or alternatively, a report includes information on prognosis, resistance, or potential or suggested therapeutic options. The report can include information on the likely effectiveness of a therapeutic option, the acceptability of a therapeutic option, or the advisability of applying the therapeutic option to a cancer patient, e.g., identified in the report. For example, the report can include information, or a recommendation, on the administration of a cancer therapy, e.g., the administration of a pre-selected dosage or in a pre-selected treatment regimen, e.g., in combination with one or more alternative cancer therapies, to the patient. The report can be delivered, e.g., to an entity described herein, within 7, 14, 21, 30, or 45 days from performing a method described herein. In some embodiments, the report is a personalized cancer treatment report.

In some embodiments, a report is generated to memorialize each time a cancer subject is tested using a method described herein. The cancer subject can be reevaluated at intervals, such as every month, every two months, every six months or every year, or more or less frequently, to monitor the subject for responsiveness to a cancer therapy and/or for an improvement in one or more cancer symptoms, e.g., described herein. In some embodiments, the report can record at least the treatment history of the cancer subject.

In one embodiment, the method further includes providing a report to another party. The other party can be, for example, the cancer subject, a caregiver, a physician, an oncologist, a hospital, clinic, third-party payor, insurance company or a government office.

### Tumors

The present disclosure provides technologies useful in the treatment of any cancer or tumor. In some embodiments, a tumor is or comprises a hematologic malignancy, including but not limited to multiple myeloma, or myeloproliferative neoplasms.

In some particular embodiments, a tumor is or comprises an advanced tumor, and/or a refractory tumor. In some embodiments, a tumor is characterized as advanced when certain pathologies are observed in a tumor (e.g., in a tissue sample, such as a biopsy sample, obtained from a tumor) and/or when cancer patients with such tumors are typically considered not to be candidates for conventional chemotherapy. In some embodiments, pathologies characterizing tumors as advanced can include tumor size, altered expression of genetic markers, invasion of adjacent organs and/ or lymph nodes by tumor cells. In some embodiments, a tumor is characterized as refractory when patients having such a tumor are resistant to one or more known therapeutic modalities (e.g., one or more conventional chemotherapy regimens) and/or when a particular patient has demonstrated resistance (e.g., lack of responsiveness) to one or more such known therapeutic modalities.

### Combination Therapy

In some embodiments, a protein therapeutic is administered in combination with a cellular therapeutic, an antibody-drug conjugate, an antibody, and/or a polypeptide. In some embodiments, the extent of tumor targeting and/or killing by a cellular therapeutic (e.g., CAR-T cell) is higher (e.g., additive or synergistic) than a level observed or measured in the absence of combined therapy.

A pharmaceutical composition comprising a protein therapeutic described herein can optionally contain, and/or be administered in combination with, one or more additional therapeutic agents, such as a cancer therapeutic agent, e.g., a chemotherapeutic agent or a biological agent. Examples of chemotherapeutic agents that can be used in combination with a protein therapeutic described herein include platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

Examples of biological agents that can be used in the compositions and methods described herein include monoclonal antibodies (e.g., rituximab, cetuximab, panetumumab, tositumomab, trastuzumab, alemtuzumab, gemtuzumab ozogamicin, bevacizumab, catumaxomab, denosumab, obinutuzumab, ofatumumab, ramucirumab, pertuzumab, ipilimumab, nivolumab, nimotuzumab, lambrolizumab, pidilizumab, siltuximab, BMS-936559, RG7446/MPDL3280A, MEDI4736, tremelimumab), enzymes (e.g., L-asparaginase), cytokines (e.g., interferons and interleukins), growth factors (e.g., colony stimulating factors and erythropoietin), cancer vaccines, gene therapy vectors, or any combination thereof.

In some embodiments, treatment methods described herein are performed on subjects for which other treatments of the medical condition have failed or have had less success in treatment through other means. Additionally, the treatment methods described herein can be performed in conjunction with one or more additional treatments of the medical condition. For instance, the method can comprise administering a cancer regimen, e.g., nonmyeloablative chemotherapy, surgery, hormone therapy, and/or radiation, prior to, substantially simultaneously with, or after the administration of a protein therapeutic described herein, or composition thereof. In certain embodiments, a subject to which a protein therapeutic described herein is administered can also be treated with antibiotics and/or one or more additional pharmaceutical agents.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXEMPLIFICATION

Exemplary amino acid and nucleotide sequences of the disclosure are listed in the following Table:

| Amino Acid SEQ ID NO. | Nucleotide SEQ ID NO. | Construct # |
|---|---|---|
| 12 | 1 | 397 |
| 13 | 2 | 493 |
| 14 | 3 | 494 |
| 15 | 4 | 522 |
| 16 | 5 | 523 |
| 17 | 6 | 524 |
| 18 | 7 | 525 |
| 19 | 8 | 536 |
| 20 | 9 | 537 |
| 21 | 10 | 538 |
| 22 | 11 | 539 |

### Example 1: Construction and Expression of Antibody-BCMA Fusion Proteins

CD38 is highly expressed on multiple myeloma (MM) and is the target of the highly successful marketed anti-CD38 mAb daratumumab. Fusion proteins comprising BCMA and an scFv that binds CD38 were constructed. BCMA was placed either N-terminal (SEQ ID NO. 13, construct #493) or C-terminal (SEQ ID NO. 14, construct #494) to the scFv. Both constructs have an added HIS tag at the C-terminus for detection and purification. Both constructs contain the BCMA extracellular domain (ECD, aa 1-54 Q02223, SEQ ID NO. 23). A signal sequence was added for the N term fusion protein and a linker, GGGGS (SEQ ID NO. 24) repeated 4 times (G4Sx4) was added between the BCMA ECD and scFv for both constructs. The anti-CD38 scFv sequence contains the variable light chain VL, a G4Sx4 linker, and the variable heavy chain VH. The anti-CD38 scFv sequences are derived from Seq ID No. 2 and Seq ID No. 27 from WO2011154453 (SEQ ID Nos. 57 and 58, respectively, herein). The constructs were chemically synthesized and cloned into the pcDNA3.1 (+) hygromycin vector (GenScript) with a His tag. Cell culture supernatants containing the BCMA fusion proteins were produced by transfecting 293T cells with the plasmid DNA using lipofectamine 2000 following the manufacturer’s protocol (Invitrogen). The supernatants were harvested 2-3 days post transfection by spinning the collected cell culture media at 12,000 rpm for 4 minutes at 4° C. to remove the cells, and then collecting the clarified media.

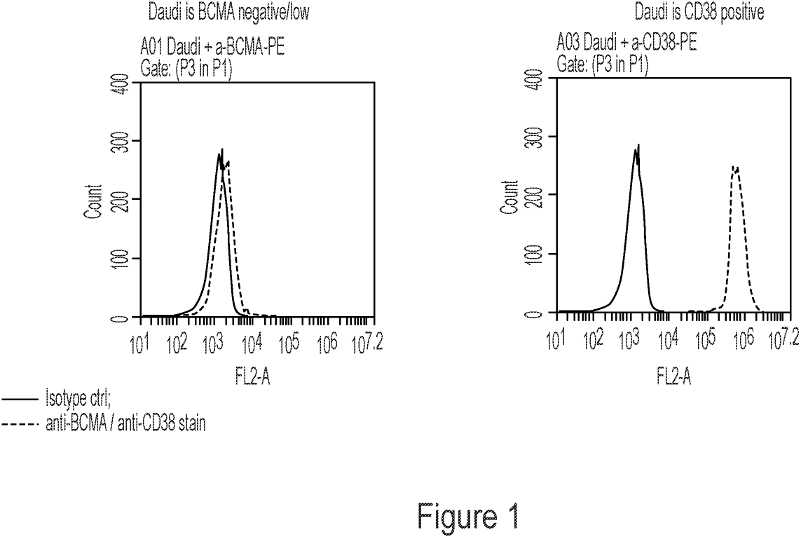

The constructs as secreted in the cell culture media were purified and evaluated for binding to the human Daudi tumor cell line. Daudi cells express very low levels of BCMA, and high levels of CD38 (FIG. 1).

Daudi cells were obtained from ATCC and cultured in RPMI containing 10% FCS. They were stained for BCMA using anti-BCMA-PE labeled antibody (BioLegend, #357504) and for CD38 using anti-CD38-PE labeled antibody (BioLegend, #356604), or stained with BCMA-containing fusion proteins as described herein. The cells were incubated with antibody or supernatant from A493 or A494 producing cells as follows. The Daudi cells were blocked with human Fc block (BD Biosciences, #BDB564129) for 10 minutes, washed and then diluted to 5×10e5 cells per 50 µl. For each sample, 50 µl of cells was aliquoted per well. For the direct stains, 5 µl of antibody in 50 µl was incubated with the cells for 30 minutes at 4° C. After washing with FACS buffer (FB: PBS, 1% BSA, 0.1% sodium azide), the cells were fixed with a final concentration of 2% paraformaldehyde in FB. For the BCMA fusion protein binding, 50 µl of A493 or A494 supernatant or 3x serial dilution of the supernatant in FB, was added and incubated for 30 minutes at 4° C. The cells were washed 2 times with FB and then stained with anti-His-PE antibody (R&D systems, #ICO5OP) or anti-BCMA-PE antibody (BioLegend, #357504) for 30 minutes at 4° C., the cells were pelleted and washed 2 times in FB and then fixed as above. The samples were analyzed on a BD Accuri 6 flow cytometer and analyzed using BD Accuri 6 software.

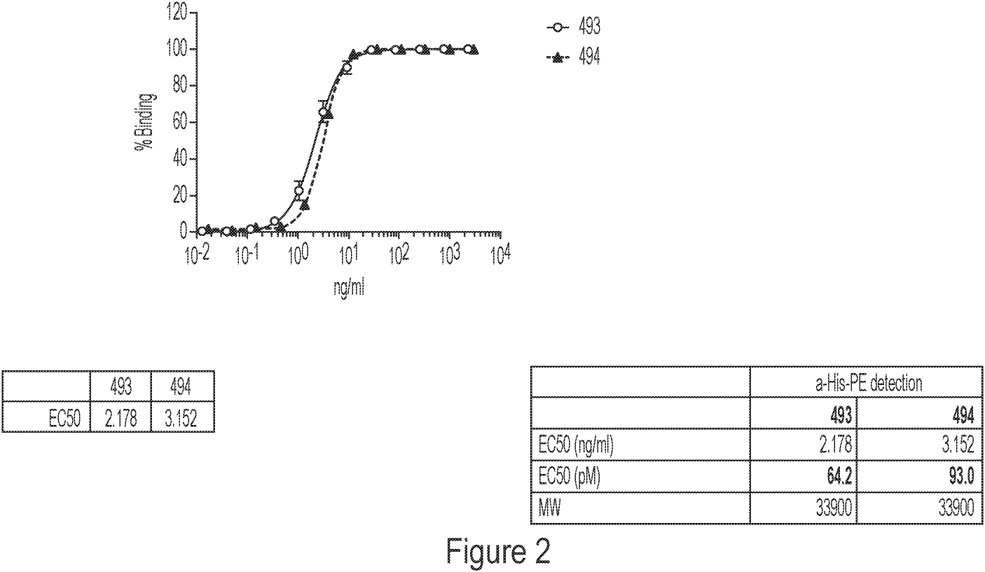

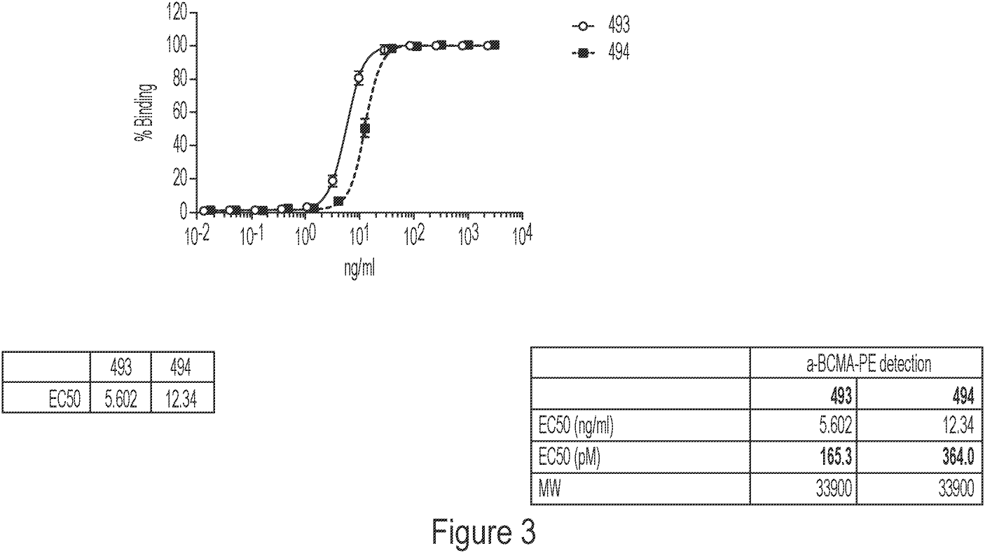

Binding of both the BCMA fusion proteins could be readily detected by either an anti-HIS tag antibody (FIG. 2), or an anti-BCMA antibody (FIG. 3).

Both BCMA-anti-CD38 fusion proteins bind in the picomolar range to CD38 on Daudi cells, with the N-terminal construct, #493, binding a little better using either detection reagent. These results demonstrate that BCMA-based fusion proteins can be readily made. While a BCMA-Fc fusion has been reported (Marsters S, et al., Current Biology 2000, 10:785-788) with BCMA placed N-terminal to the Fc, no other fusions between BCMA and other proteins or antibody fragments have been reported in the literature, to our knowledge, and none with BCMA placed C-terminal to any protein.

### Example 2: Binding Data on BCMA-Anti-GPRC5D Binding Proteins

Recent work has identified a new target highly expressed on MM, a GPCR called GPRC5D (Smith et al., Sci. Transl. Med. 11, eaau7746 (2019)). Further, a series of useful scFvs to GPRC5D has also been described (WO2016090312A1). Based on those disclosures, four anti-GPRC5D scFvs (constructs 522-525; SEQ ID NO. 15-18, respectively) expression constructs were chemically synthesized from back translated sequences (SEQ ID NO. 114, 115, 116 and 117 from Brentjen et al. WO2016090312A1) and cloned into pcDNA3.1 (+) hygro vector by GenScript. The expression constructs contain the scFvs encoded as VL-G4Sx3-VH-His.

The scFv expression constructs were transiently expressed in HEK293 cells, and the binding of the supernatants evaluated on GPRC5D-expressing HEK293 cells. All four scFvs bound well to GPRC5D-expressing cells (not shown). Therefore, four new fusion protein constructs were made comprising these scFvs fused to the BCMA extracellular domain (ECD) placed C-terminal to the scFv, with a HIS tag added at the C-terminus (constructs 536-539, SEQ ID NO. 19-22 respectively). To make the C-terminal BCMA ECD fusions, construct A494 was amplified to obtain the vector backbone plus the BCMA ECD. PCR fragments were generated from the anti-GPRC5D scFv templates, A522-525, and these were assembled with the A494 backbone to generate A536-539 using a One Step Seamless Cloning Mix (CoWin Biosciences, CW3034S). These expression constructs encode anti-GPRC5D scFv-G4Sx3 linker-BCMA ECD-His.

The expression constructs were transiently expressed in HEK cells. The fusion protein expression levels in the cell culture supernatants were quantified by ELISA analysis. Briefly, a 96 well plate was coated overnight at 4° C. with 1.0 µg/ml PE anti-human BCMA antibody (Biolegend, #357504) in 0.1 M carbonate, pH9.5 for A493 and A494, or 1.0 µg/ml anti-human BCMA antibody (Biolegend, #357502) for A536, A537, A538, A539 and A540. The plate was blocked with 0.3% non-fat milk in TBS for 1 hour at room temperature. After washing in TBST (0.1 M Tris, 0.5 M NaCl, 0.05% Tween-20) 3 times, the fusion protein supernatant was titrated using 3-fold dilutions in 1% BSA in TBS and incubated 1 hour at room temperature. Purified BCMA-His (Acro Biosystems, #BCA-H522y) starting at 1 µg/ml with 3-fold dilutions was used as a standard curve. Then, 100 µl HRP-anti-his antibody (Biolegend, #652504) was added at a 1:2000 dilution and incubated at room temperature in the dark for 1 hour. Then, 1-Step Ultra TMB-ELISA solution (Thermo Fisher) was added to develop the peroxidase signal, and the plate read at 405 nm. The standard curve was fit using a four-parameter logistic (4PL) regression to calculate the unknown supernatant concentration.

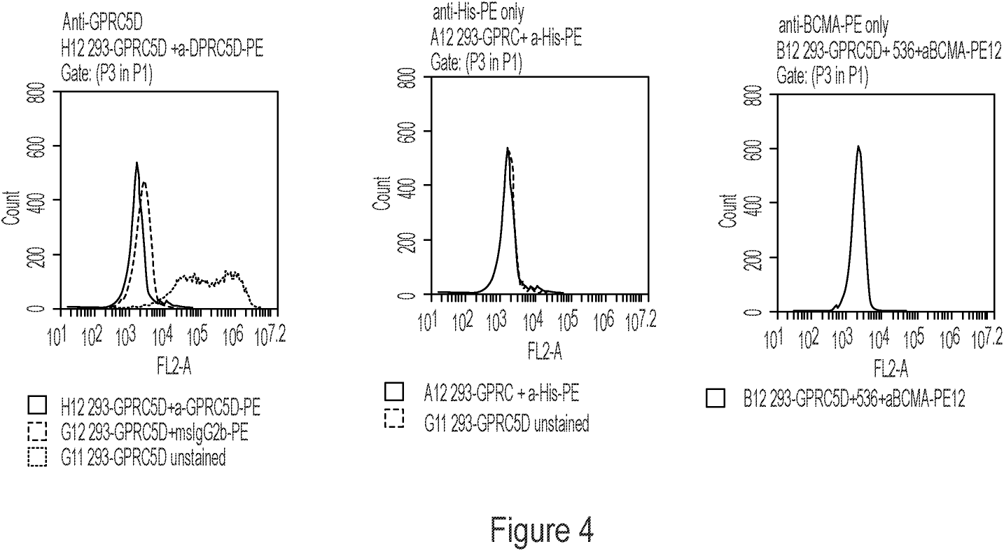

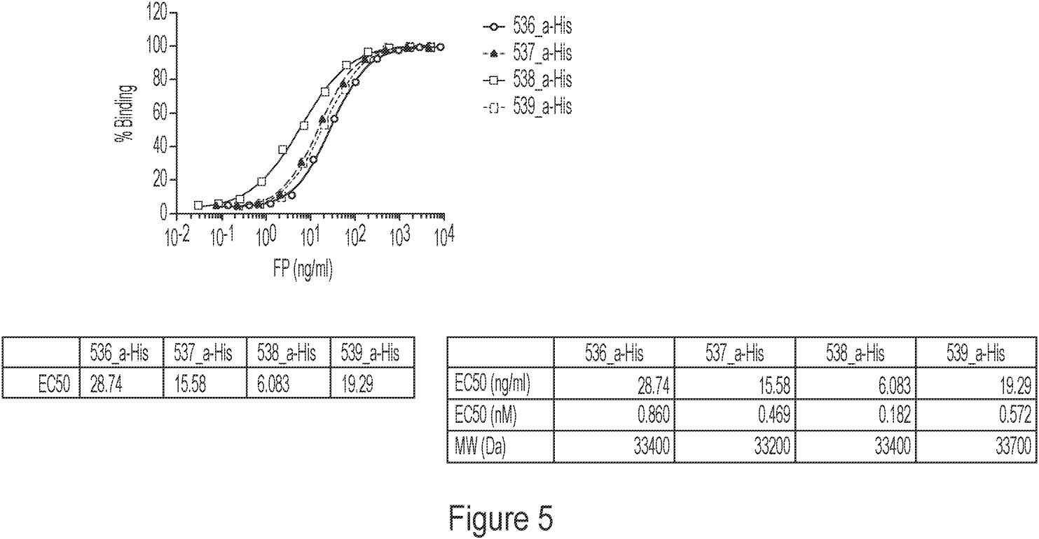

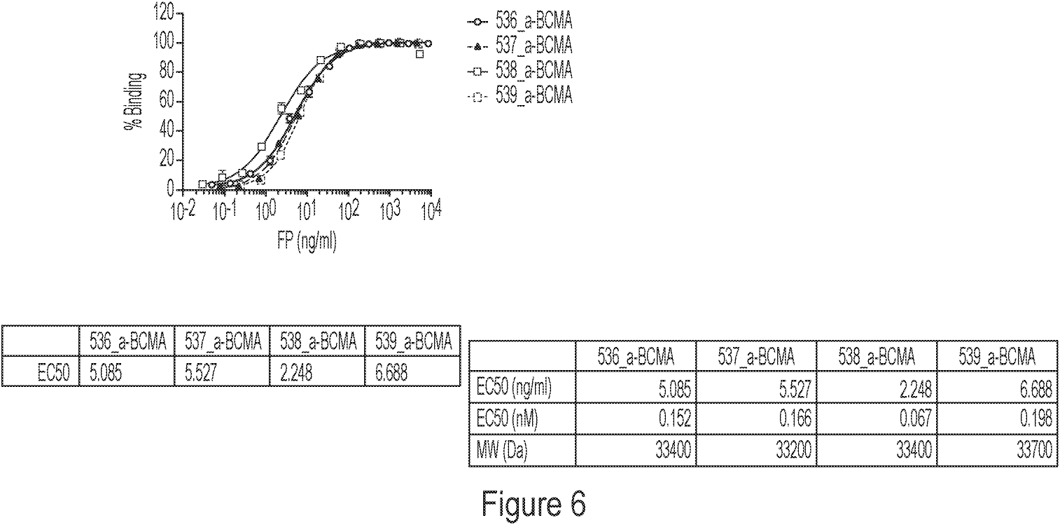

Supernatants from the fusion protein expressing HEK cells were evaluated for binding to HEK293 cells transfected with a GPRC5D expression plasmid. Binding of all four constructs was readily detected to highly expressing GPRC5D cells (FIG. 4), using either an anti-HIS tag antibody (FIG. 5) or an anti-BCMA antibody (FIG. 6). Sub-nanomolar binding was detected in all four constructs.

Thus, the BCMA ECD fused to the C-termini of four different scFvs directed to the GPCR called GPRC5D, an antigen highly upregulated on human MM, were well expressed and bound to HEK293 cells that were transiently expressing GPRC5D with sub-nanomolar potency. This provides a second example of a BCMA-containing fusion protein capable of binding effectively to an antigen highly expressed on human multiple myeloma, and of a BCMA-containing fusion protein with BCMA placed C-terminal to an scFv.

### Example 3: A CAR-T Cell Directed to BCMA

Numerous CAR-T cells directed to BCMA-expressing cells have been published in the literature, with multiple examples in clinical trials (Carpenter et al., Clin Cancer Res. 2013 Apr 15;19(8):2048-60; Friedman et al. 2018 Hum Gene Ther.: 29(5): 585-601. We developed a BCMA-targeted CAR-T cells as described in US 2012/0082661 A1 and Carpenter et al., Clin Cancer Res. 2013 Apr 15;19(8):2048-60.

The CAR BCMA construct contains the anti-BCMA CAR sequence VL-VH (anti-BCMA CAR consisting of the heavy and light chains sequences from the murine anti-BCMA antibody C11D5.3 (SEQ ID NO. 3 and 4 from US2012/0082661A1); our SEQ ID NO. 12 (#397)) separated by linker GSTSGSGKPGSGEGSTKG (Cooper et al. 2003 Blood 101: 1637-1644). The construct also includes a FLAG-tag, a CD28 linker, transmembrane domain and intracellular domain (aa 114-220 P10747), and the 4-1BB (aa 214-255 Q07011) and CD3 zeta intracellular domains (aa 52-164 P20963). The anti-BCMA scFv sequence was chemically synthesized and cloned into a modified lentiviral plasmid pCDH-EF1a (Systems Biosciences, #CD514B-1) containing an MSCV promoter. After transformation into NEB stable competent cells, a correct isolate was identified and a large-scale plasmid preparation made using an endo-free maxiprep kit (CoWin Biosciences). For the production of lentiviral particles, the following Aldevron packaging plasmids and the transgene plasmid (for each T75 flask) were combined and mixed gently in 1.5 mL Opti-MEM (Invitrogen): 7 µg of the BCMA CAR plasmid, 5.7 µg of the VSVG plasmid (5037-10 pALD-VSV-G-A), 7 µg of the GagPol plasmid (5035-10 pALD-GagPol-A), and 2.8 µg of the Rev plasmid (5033-10 pALD-Rev-A). Then 45 µL of Trans-IT (Mirus, #MIR6604) transfection reagent was added and mixed. The mixture was allowed to complex at room temperature for 20 minutes. The recipient cells, 293FT, were plated in DMEM containing 10% FBS prior to transfection to obtain ~70% confluency. Prior to the transfection, the growth medium on the 293FT cells was replaced with 10 mL Opti-MEM. The DNA/Trans-IT mixture was added dropwise into the T25 flask. The flasks were incubated for 24 hours and the media replaced with antibiotic free DMEM+10% FBS daily for 3 days. Harvested media was stored at 4° C. The viral particles were precipitated by adding 5X PEG-IT (Systems Biosciences, LV825A-1) to the supernatant, mixing and incubating at 4° C. for 72 hrs. The mixture was centrifuged at 3000 RCF for 30 minutes, the residual supernatant removed and the pellet resuspended in 200 µl PBS and stored at -80° C. The viral particles were titered on SupT1 cells by determining CAR expression after 3 days using anti-Flag antibody staining and flow cytometric analysis.

For production and characterization of BCMA CAR, PBMCs from normal human donors were collected and CD3-positive human primary T cells were isolated using magnetic bead technology (MACs^{tm}). Purified CD3-positive human primary T cells were cultivated in ImmunoCult-XF T cell expansion medium (serum/xeno-free) supplemented with 50 IU/ml IL-2 at a density of 3 × 10⁶ cells/mL, activated with CD3/CD28 T cell Activator reagent (STEMCELL Technologies) and transduced on day 1 with the BCMA CAR397 lentiviral particles, using a volume determined after titering, in the presence of 1X Transdux (from SBI). The cells were propagated until harvest on day 10. Post-expansion, CAR T cells were stained with anti-FLAG antibody to measure CAR expression. Briefly, 100,000 cells were incubated with anti-FLAG antibody (Thermo Fisher), diluted 1:100 in PBS for 60 minutes at 10° C., followed by anti-rabbit APC (1:100 dilution, Thermo Fisher). In addition, CAR T cells were stained for CD8 using anti-CD8 MEM-31 antibody, diluted 1:100 (Invitrogen). Cells were resuspended in PBS and fixed at a final concentration of 2% paraformaldehyde. Cell populations were analyzed using a BD Accuri C6 flow cytometer.

Direct killing of a BCMA positive cell line, H929, was shown with the BCMA CAR397. H929 cells (ATCC) were grown in RPMI1640 containing 10% FCS. A H929 cell line expressing luciferase was generated by transduction with a lentivirus (Gencopoeia, #LPP-HLUC-Lv105-100-C) and selection with puromycin. Cells (1 × 10e4/50 µL/well) were seeded in a 96 well round bottom plate in RPMI containing 10% FBS without antibiotics (RPMI/FBS). The BCMA CAR397 or donor-matched untransduced T cells were thawed and washed once with RPMI/FBS via centrifugation at 550 RCF for 10 minutes. The CAR T cells were added to the wells in 50 µL to give a CAR:target cell ratio of 30:1, 10:1, 5:1 or 1:1 respectively. The plates were incubated at 37° C. for 48 hours. The plate was centrifuged at 550 RCF for 5 minutes, the pellet was rinsed with PBS, and spun again. Then, 20 µL of 1 × lysis buffer (Promega, #E1500) was added to the pellet, and the lysate was transferred into a 96 well opaque tissue culture plate (Fisher Scientific, #353296). The plates were read in a luminometer with an injector to dispense the substrate (Promega, #E1500). The percent killing was calculated based upon the average loss of luminescence of the experimental vs the control (untreated) cells.

In the case of cytotoxicity using the BCMA ECD fusion proteins, dilutions of the bridging protein #538 at 500 µg/ml and 100 µg/ml were made in 25 µL RPMI/FBS and added to 1×10e4 per well of 293T expressing GPRC5D cells. The cells express the target, GPRC5D, but not BCMA, so the efficacy of the bridging protein can be measured. The GPRC5D cell line was produced by transfection of a cDNA (GenScript, OHu02831D), into 293T cells expressing luciferase using lipofectamine and following the manufacturer’s protocol (Invitrogen). Clones were isolated after G418 selection and then used in cytotoxicity assays. The BCMA CAR397 cells were thawed and washed once with RPMI containing 10% FBS via centrifugation at 550 RCF for 10 minutes. The CAR T cells were incubated in RPMI/FBS for 6 hours at 37° C. before adding to the target cells. The CAR T cells were added to the wells in 25 µL to give a CAR:target cell ratio of 10:1. The rest of steps were the same for direct CAR killing as described herein.

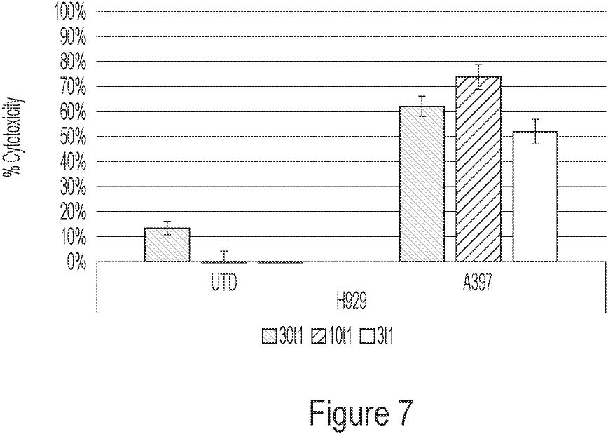

The BCMA directed CAR-T showed it capable of killing human H929 MM cells in culture as shown in FIG. 7.

### Example 4: Use of BCMA-Bridging Proteins to Direct Killing of BCMA-Negative Cells

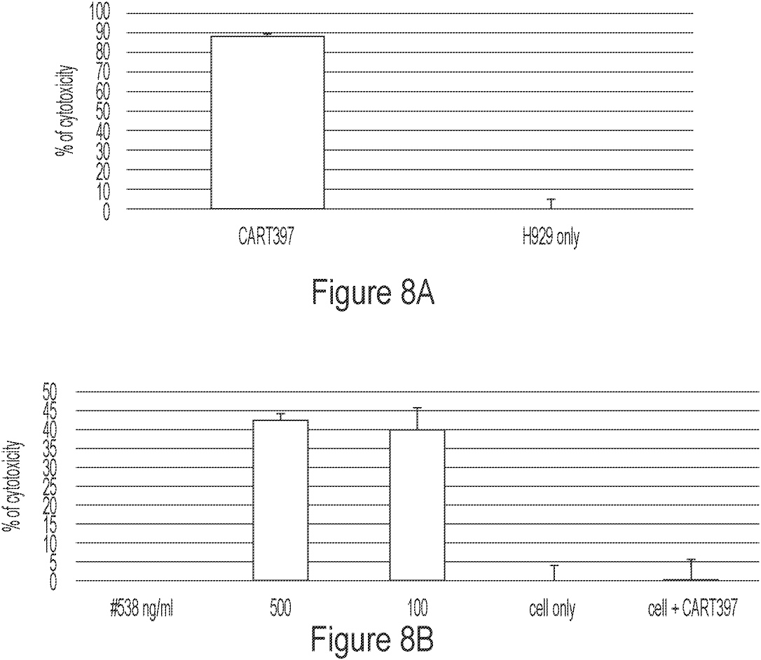

293T cells, which are GPRC5D, CD38, and BCMA negative, expressing luciferase were transfected with GPRC5D cDNA purchased from Genscript. Clones expressing GPRC5D were transiently transfected (see FIG. 4). Two dilutions of a fusion protein construct (#538), comprising an scFv that recognizes GPRC5D fused to BCMA, was added to the 293T cells expressing GPRC5D. A cytotoxicity assay, as described herein (see Example 3), was performed to determine the ability of the fusion protein to bridge the 293T cell with the antigen bound by the antigen binding polypeptide (GPRC5D) and the CAR T cell recognizing the polypeptide antigen (BCMA), thus facilitating killing of cells that lack BCMA by a BCMA targeting CAR T cell. As shown in FIG. 8B, GPRC5D-expressing cells were killed by the BCMA-CART cells only in the presence of the BCMA-bridging protein. H929 cells expressing BCMA served as a positive control for the killing activity of construct #538

### Example 5: Generation of a BCMA-binding Proteins With an Extended Half-life, and Assessment of Binding and Cytotoxicity

Fusion proteins comprising an anti-CD38 scFv; BCMA; and an albumin-binding domain are generated as described herein. Fusion proteins comprising an anti- GPRC5D scFv; BCMA; and an albumin-binding domains are generated as described herein. The fusion protein will be linked to the albumin binding domain sequence Alb8 at the N- or C-terminus of the fusion protein, or centrally in the fusion protein. The Alb8 will be derived from amino acid sequence GenBank entry AUE82538 (aa 1-115).

The fusion proteins will be evaluated for binding to cells expressing CD38 or GPRC5D. The fusion proteins will be evaluated for their ability to bridge BCMA directed CAR-T cells to CD38-positive cells and/or GPRC5D-positive cells in vitro. The fusion proteins will be evaluated for their ability to trigger cytotoxic activity of BCMA directed CAR-T cells against CD38-positive cells and/or GPRC5D-positive cells in vitro. The plasma half-life of the fusion proteins will be tested in vivo in normal mice.

### Example 6: Use of BCMA-Containing Fusion Proteins to Direct Killing of BCMA-low or BCMA-Negative Cells in Vivo

### Pharmacokinetics to Assess the BCMA-Anti-GPRC5D-Anti-Albumin Fusion Proteins

Ten female NOD-scid IL2R gamma-null (NSG) mice, 6-8 weeks of age, are ordered from Jackson Laboratories and used to determine the PK of the proteins. The mice are injected IV at 5 mg/kg. Blood is sampled at time points 0, 30 minutes, 90 minutes, 6 hours, 24 hours, 48 hours, 72 hours, 9 hours and 120 hours. Whole blood is collected via tail bleed. Each mouse is bled 2 times in life (maximum volume = 100 µL) and once at the terminal bleed. The collected blood is placed into EDTA K3 tubes (Sarstedt, #411504105). The blood is processed by spinning the samples in a microcentrifuge at 10 minutes at 8500 rpm. Plasma is then transferred to a 1.5 mL Eppendorf and frozen until study termination. An ELISA to measure the BCMA bridging proteins in the serum is carried out as described for the titer ELISA.

### Efficacy Study to Assess the Activity of the BCMA-Anti-GPRC5D-Anti-Albuminfusion Proteins

OPM-2 cells (DSMZ cell bank), stably expressing firefly luciferase (OPM-2-luc), or a similar human myeloma cell line, are used as in the myeloma model as described in Smith, EL et al. 2019. Briefly, OPM-2-luc cells (1 × 10e6) are injected into female NSG mice (Jackson Laboratory) via the tail vein and allowed to grow for about 14 days. The mice are randomized into groups and treated with 1 × 10e7 CAR-T cells expressing a BCMA scFv (e.g. CAR-397 or a similar CAR-T cell targeting BCMA) with or without added BCMA bridging protein, e.g. BCMA ECD-anti-GPRC5D scFv-alb8 bridging protein. The CAR-397 or similar CAR-T cells are administered via tail vein injection and the bridging protein by intraperitoneal or intravenous injection. The bridging protein will be dosed twice a week at 100 µg/injection or at a concentration higher or lower than 100 µg, as guided by the experimental results. A CAR-GPRC5D is used as a positive control (Smith et al. 2019), and either no CARs T cells or donor-matched untransduced T cells are used as negative controls. The luciferase level of the tumors is monitored twice weekly and the mice are sacrificed when the tumor burden reaches the limit outlined in our IACUC protocol and guidelines. A similar protocol using a CD38-positive BCMA-low cell line, e.g. Daudi cells, can be used to assess the efficacy of a BCMA-anti-CD38 binding protein, as described, for example, in Example 1.

### Example 7: Construction of a Bivalent scFv-BCMA Fusion Protein Based on Low Affinity Binders of CD38

Placement of two low affinity scFvs or VHHs in series allows the resultant bivalent fusion protein as described herein to bind a target cell with high affinity, but only if the target antigen is highly expressed on the cell surface. scFvs with low affinity for CD38 have already been identified, using light chain VL shuffling with the heavy chain VH of scFv 028 (see for example Drent et al., Molecular Therapy Vol. 25 No 8 Aug. 2017). These scFvs (with much lower affinities) are linked together in a bivalent format and assessed for high avidity binding to a CD38-hi cell line (e.g., expressing a high level of CD38) .

First, four 028-based scFvs determined to be of particularly low affinity following light chain shuffling (as described in Drent et al, supplement Table S1, light chains A1, A3, B1, and B3) are constructed using standard methods (e.g., as described in Example 1). All constructs (see SEQ ID Nos.: 25-32) are chemically synthesized and include a HIS tag at the C-terminus for detection and purification. Constructs are expressed transiently in HEK cells, supernatants harvested, and their binding to CD38 high-expressing Daudi cells is assessed by FACS, as described in Example 1.

Second, with binding to CD38 with low affinity confirmed, a bivalent form of each scFv is generated using a standard linker of various lengths, for example GGGGS (SEQ ID NO. 24) repeated between 2 and 5 times, although other linkers can be used, between the two scFvs, again with an added HIS tag at the C-terminus for detection and purification. Constructs (see SEQ ID Nos. 33-40) are expressed transiently in HEK cells, supernatants harvested, and their binding to CD38-expressing cells assessed by FACS, as described in Example 1. Binding of the constructs is compared on CD38-hi Daudi cells as well as on CD38-lo U937 cells, Molm14 cells or CD38 transfected 293T cells selected for low expression of CD38.

Third, bivalent constructs which bind with high apparent affinity to Daudi CD38-hi cells, but much less well to U937 or Molm14 CD38-lo cells, are further evaluated as BCMA fusion proteins. BCMA will be placed either N-terminal (see SEQ ID NOs. 43, 44, 47, 48, 51, 52, 55, 56) or C-terminal (SEQ ID NOs. 41, 42, 45, 46, 49, 50, 53, 54) to the scFv-linker-scFv construct. In addition, BCMA placed centrally between the two scFvs (i.e. as the linker or part of the linker) is also evaluated. All constructs have an added HIS tag at the C-terminus for detection and purification, and contain the BCMA extracellular domain (ECD, aa 1-54 Q02223, SEQ ID NO. 23). Again, constructs are expressed transiently in HEK cells, supernatants harvested, and their binding to CD38-expressing cells assessed by FACS, as described in Example 1. Their binding is compared on CD38-hi Daudi cells as well as on CD38-lo U937 or Molm14 cells, as described in Example 1. Daudi cells are obtained from ATCC and cultured in RPMI containing 10% FCS. U937 cells are obtained from ATCC and the Molm14 cells from DSMZ cell culture collection and cultured in RPMI containing 10% FCS.

By this means bivalent BCMA fusion proteins are identified using low affinity variants of scFv 028, which selectively bind CD38-hi cells, e.g. myeloma tumor cells, as compared to CD38-lo cells, e.g. normal leukocyte subsets known to express low levels of CD38.

BCMA fusion protein PKs and efficacy in vivo are assessed as described in Example 6.

### Example 8: Construction of Bivalent VHH-BCMA Fusion Proteins Based on Low Affinity Llama VHHs

Low affinity llama VHH CD38 binders are linked in a bivalent format to generate high avidity binders selective for CD38-hi cells. In order to generate anti-CD38 antibodies, one or more adult llamas are immunized three times for a total of 600 µg per llama with a His tagged extracellular domain of CD38 (AcroBiosystems) in Complete Freund’s adjuvant by ProSci, Inc. (Poway, CA). A phagemid library is generated from the llama PBMCs and screened by panning using biotinylated CD38 ECD. Positive clones are screened by ELISA as follows. Plates are coated with 1 µg/mL human CD38 ECD in PBS (overnight at 4° C.), and then blocked with 5% milk/PBST (PBS-Tween) for 2 hours at room temperature. E.coli extracts, containing the llama sdAbs, are diluted 1:1 in blocking buffer (PBS/1% BSA) and allowed to bind to the plate for 1 hour at room temperature. After washing with PBST, the plate-bound sdAbs are detected with a mouse anti-myc-tag monoclonal antibody (mAb) for 1 hour, followed by goat anti-mouse IgG-HRP for 1 hour. Both incubations are performed in blocking buffer, followed by 5 washes with PBST. The bound HRP is detected using peroxidase enzymatic detection. The sequence of positive clones is determined, and a small number are purified from lysates using an anti-His Nickel NTA column following the manufacturer’s protocol (Qiagen, Germantown, MD).

The purified sdAbs are screened for binding to CD38-hi Daudi cells. Briefly, Daudi cells (2.5×10^5) are blocked with Fc block (BD Pharmingen) for 10 minutes on ice. Then, the purified sdAb dilutions are added, starting at 3 µg/ml with 3 fold serial dilutions in FACS buffer (PBS + 1% BSA + 0.1% sodium azide), and incubated for 30 minutes on ice. The samples are washed 2 times with FACS buffer and then incubated with anti-His-PE (5 µl per sample, R&D Systems) for 30 minutes on ice. Next, the samples are washed 2 times with FACS buffer and then fixed with 2% paraformaldehyde. The samples are analyzed by flow cytometry.

sdAbs which bind to Daudi cells, and with affinities of 100 nM to 500 nM, are evaluated in a bivalent format exactly as described in Example 7. Briefly, sdAbs are placed in series, connected by a standard linker of various lengths, and a C-terminal HIS tag added. After expression in HEK cells, supernatants are assessed for selective binding to CD38-hi Daudi versus CD38-lo U937 or Molm14 cells. A BCMA ECD is added to those constructs showing preferential binding to Daudi cells, and these constructs are re-assessed for binding to both cell types, but with detection via anti-BCMA antibodies (se Example 1), resulting in BCMA-anti-CD38 bivalent fusion proteins selective for CD38-hi myeloma cells versus CD38-lo cells.

A selected number of BCMA-anti-CD38 fusion proteins are constructed with an added albumin-binding domain as described in example 5. PK in vivo, and efficacy in vivo against CD38-hi Daudi or similar tumor cells, is assessed as described in Example 6.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

### LISTING OF SEQUENCES

SEQ ID NO. 1

ATGGAGACAGACACACTCCTGCTATGGGTACTGCTGCTCTGGGTTCCAGG
TTCCACTGGTGACATCGTGCTGACCCAGAGCCCCCCCAGCCTGGCCATGA
GCCTGGGCAAGAGGGCCACCATCAGCTGCAGGGCCAGCGAGAGCGTGACC
ATCCTGGGCAGCCACCTGATCCACTGGTACCAGCAGAAGCCCGGCCAGCC
CCCCACCCTGCTGATCCAGCTGGCCAGCAACGTGCAGACCGGCGTGCCCG
CCAGGTTCAGCGGCAGCGGCAGCAGGACCGACTTCACCCTGACCATCGAC
CCCGTGGAGGAGGACGACGTGGCCGTGTACTACTGCCTGCAGAGCAGGAC
CATCCCCAGGACCTTCGGCGGCGGCACCAAGCTGGAGATCAAGGGCAGCA
CCAGCGGCAGCGGCAAGCCCGGCAGCGGCGAGGGCAGCACCAAGGGCCAG
ATCCAGCTGGTGCAGAGCGGCCCCGAGCTGAAGAAGCCCGGCGAGACCGT
GAAGATCAGCTGCAAGGCCAGCGGCTACACCTTCACCGACTACAGCATCA
ACTGGGTGAAGAGGGCCCCCGGCAAGGGCCTGAAGTGGATGGGCTGGATC
AACACCGAGACCAGGGAGCCCGCCTACGCCTACGACTTCAGGGGCAGGTT
CGCCTTCAGCCTGGAGACCAGCGCCAGCACCGCCTACCTGCAGATCAACA
ACCTGAAGTACGAGGACACCGCCACCTACTTCTGCGCCCTGGACTACAGC
TACGCCATGGACTACTGGGGCCAGGGCACCAGCGTGACCGTGAGCAGCGA
CTACAAAGACGATGACGACAAGATTGAAGTTATGTATCCTCCTCCTTACC
TAGACAATGAGAAGAGCAATGGAACCATTATCCATGTGAAAGGGAAACAC
CTTTGTCCAAGTCCCCTATTTCCCGGACCTTCTAAGCCCTTTTGGGTGCT
GGTGGTGGTTGGTGGAGTCCTGGCTTGCTATAGCTTGCTAGTAACAGTGG
CCTTTATTATTTTCTGGGTCCGCAGTAAGAGGAGCAGGCTCCTGCACAGT
GACTACATGAACATGACTCCCCGCCGCCCCGGGCCCACCCGCAAGCATTA
CCAGCCCTATGCCCCACCACGCGACTTCGCAGCCTATCGCTCCAAACGGG
GCAGAAAGAAACTCCTGTATATATTCAAACAACCATTTATGAGACCAGTA
CAAACTACTCAAGAGGAAGATGGCTGTAGCTGCCGATTTCCAGAAGAAGA
AGAAGGAGGATGTGAACTGAGAGTGAAGTTCAGCAGGAGCGCAGACGCCC
CCGCGTACCAGCAGGGCCAGAACCAGCTCTATAACGAGCTCAATCTAGGA
CGAAGAGAGGAGTACGATGTTTTGGACAAGAGACGTGGCCGGGACCCTGA
GATGGGGGGAAAGCCGCAGAGAAGGAAGAACCCTCAGGAAGGCCTGTACA
ATGAACTGCAGAAAGATAAGATGGCGGAGGCCTACAGTGAGATTGGGATG
AAAGGCGAGCGCCGGAGGGGCAAGGGGCACGATGGCCTTTACCAGGGTCT
CAGTACAGCCACCAAGGACACCTACGACGCCCTTCACATGCAAGCCCTGC
CCCCTCGC

SEQ ID NO. 2

ATGAGGCTGCTGGTGCTGCTGTGGGGCTGCCTGCTGCTGCCCGGCTACGA
GGCCATGCTGCAGATGGCCGGCCAGTGCAGCCAGAACGAGTACTTCGACA
GCCTGCTGCACGCCTGCATCCCCTGCCAGCTGAGGTGCAGCAGCAACACC
CCCCCCCTGACCTGCCAGAGGTACTGCAACGCCAGCGTGACCAACAGCGT
GAAGGGCACCAACGCCGGCGGCGGCGGCAGCGGCGGCGGCGGCAGCGGCG
GCGGCGGCAGCGGCGGCGGCGGATCCGACATCCAGATGACCCAGAGCCCC
AGCAGCCTGAGCGCCAGCGTGGGCGACAGGGTGACCATCACCTGCAGGGC
CAGCCAGGGCATCAGGAGCTGGCTGGCCTGGTACCAGCAGAAGCCCGAGA
AGGCCCCCAAGAGCCTGATCTACGCCGCCAGCAGCCTGCAGAGCGGCGTG
CCCAGCAGGTTCAGCGGCAGCGGCAGCGGCACCGACTTCACCCTGACCAT
CAGCAGCCTGCAGCCCGAGGACTTCGCCACCTACTACTGCCAGCAGTACA
ACAGCTACCCCCTGACCTTCGGCGGCGGCACCAAGGTGGAGATCAAGGGC
GGCGGCGGCAGCGGCGGCGGCGGCAGCGGCGGCGGCGGCAGCGGCGGCGG
CGGCAGCCAGGTGCAGCTGGTGCAGAGCGGCGCCGAGGTGAAGAAGCCCG
GCAGCAGCGTGAAGGTGAGCTGCAAGGCCTTCGGCGGCACCTTCAGCAGC
TACGCCATCAGCTGGGTGAGGCAGGCCCCCGGCCAGGGCCTGGAGTGGAT
GGGCAGGATCATCAGGTTCCTGGGCATCGCCAACTACGCCCAGAAGTTCC
AGGGCAGGGTGACCCTGATCGCCGACAAGAGCACCAACACCGCCTACATG
GAGCTGAGCAGCCTGAGGAGCGAGGACACCGCCGTGTACTACTGCGCCGG
CGAGCCCGGCGAGAGGGACCCCGACGCCGTGGACATCTGGGGCCAGGGCA
CCATGGTGACCGTGAGCAGCCACCACCACCACCACCAC

SEQ ID NO. 3

ATGAGGCTGCTGGTGCTGCTGTGGGGCTGCCTGCTGCTGCCCGGCTACGA
GGCCGACATCCAGATGACCCAGAGCCCCAGCAGCCTGAGCGCCAGCGTGG
GCGACAGGGTGACCATCACCTGCAGGGCCAGCCAGGGCATCAGGAGCTGG
CTGGCCTGGTACCAGCAGAAGCCCGAGAAGGCCCCCAAGAGCCTGATCTA
CGCCGCCAGCAGCCTGCAGAGCGGCGTGCCCAGCAGGTTCAGCGGCAGCG
GCAGCGGCACCGACTTCACCCTGACCATCAGCAGCCTGCAGCCCGAGGAC
TTCGCCACCTACTACTGCCAGCAGTACAACAGCTACCCCCTGACCTTCGG
CGGCGGCACCAAGGTGGAGATCAAGGGCGGCGGCGGCAGCGGCGGCGGCG
GCAGCGGCGGCGGCGGCAGCGGCGGCGGCGGCAGCCAGGTGCAGCTGGTG
CAGAGCGGCGCCGAGGTGAAGAAGCCCGGCAGCAGCGTGAAGGTGAGCTG
CAAGGCCTTCGGCGGCACCTTCAGCAGCTACGCCATCAGCTGGGTGAGGC
AGGCCCCCGGCCAGGGCCTGGAGTGGATGGGCAGGATCATCAGGTTCCTG
GGCATCGCCAACTACGCCCAGAAGTTCCAGGGCAGGGTGACCCTGATCGC
CGACAAGAGCACCAACACCGCCTACATGGAGCTGAGCAGCCTGAGGAGCG
AGGACACCGCCGTGTACTACTGCGCCGGCGAGCCCGGCGAGAGGGACCCC
GACGCCGTGGACATCTGGGGCCAGGGCACCATGGTGACCGTGAGCAGCGG
CGGCGGCGGCAGCGGCGGCGGCGGCAGCGGCGGCGGCGGCAGCGGCGGCG
GCGGATCCATGCTGCAGATGGCCGGCCAGTGCAGCCAGAACGAGTACTTC
GACAGCCTGCTGCACGCCTGCATCCCCTGCCAGCTGAGGTGCAGCAGCAA
CACCCCCCCCCTGACCTGCCAGAGGTACTGCAACGCCAGCGTGACCAACA
GCGTGAAGGGCACCAACGCCCACCACCACCACCACCAC

SEQ ID NO. 4

ATGGAGACCGACACACTGCTGCTGTGGGTGCTGCTGCTGTGGGTGCCCGG
CAGCACAGGACAGTCCGTGCTGACCCAGCCAGCCTCCGTGTCTGGCAGCC
CAGGCCAGTCTCTGACCATCAGCTGCACCGGCACATCTAACGATGTGGGC
GCCTACAAGTATGTGAGCTGGTATCAGCAGTATCCCGGCAAGGCCCCTAA
GCTGATCCTGTACGACGTGTTCAAGAGGCCTTCCGGCGTGTCTAACCGCT
TTTCCGGCTCTAAGAGCGATAATACAGCCTCCCTGACCATCTCTGGACTG
CAGGCAGAGGACGAGGCAGATTACTATTGCTTCAGCCTGACAAGCTCCAA
CACCTACGTGTTTGGCACCGGCACAAAGGTGACCGTGCTGGGCTCCCGGG
GCGGAGGAGGCAGCGGAGGAGGAGGCTCCGGCGGCGGCGGCTCTCTGGAG
ATGGCACAGATGCAGCTGGTGCAGAGCGGAGCAGAGGTGAAGAAGCCTGG
AGCCAGCGTGAAGGTGTCCTGTAAGGCCTCTGGCTACACATTCAATCGGT
ATGCCATCACCTGGGTGAGACAGGCACCAGGACAGGGCCTGGAGTGGATG
GGCTGGATCAGCGCCTACAACGGCAATTCCCACTATGCCCAGAAGCTGCA
GGGCCGGGTGACAATGACCACAGACACCTCCACAGGCACCGCCTACATGG
AGCTGCGGAGACTGAGGTCTGACGATACCGCCGTGTACTATTGTGCCCGC
ATGGCCTATGATTCCTGGGGCCAGGGCACACTGGTGACCGTGTCTAGCCA
CCACCACCACCACCAC

SEQ ID NO. 5

ATGGAGACCGACACACTGCTGCTGTGGGTGCTGCTGCTGTGGGTGCCCGG
CTCTACCGGACAGAGCGTGCTGACACAGCCACCTTCCGCCTCTGGCACCC
CTGGACAGCGGGTGACAATCAGCTGCTCCGGCAGCCGGAGCAACGTGGGA
GGCAATTACGTGTTCTGGTATCAGCAGGTGCCAGGAGCAACCCCAAAGCT
GCTGATCTACCGGTCCAACCAGAGACCTTCTGGCGTGCCAGATCGGTTTG
CAGGCTCCAAGTCTGGCAGCTCCGCCTCCCTGGCCATCTCTGGACTGAGA
AGCGAGGACGAGGCCGATTACTATTGCGCCACCTGGGACGATAGCCTGTC
CGGCTTCGTGTTTGGCACCGGCACAAAGGTGACAGTGCTGGGCAGCCGGG
GCGGAGGAGGCAGCGGAGGAGGAGGCTCCGGCGGCGGCGGCTCTCTGGAG
ATGGCCGAGGTGCAGCTGGTGGAGTCCGGAGGAGGACTGGTGAAGCCAGG
AGGCTCCCTGAGGCTGTCTTGTGCAGCCAGCGGCTTCACCTTTAGCGACT
ACTATATGTCCTGGATCAGACAGGCACCAGGCAAGGGCCTGGAGTGGGTG
TCTTACATCTCTAGCTCCGGCAGCACCATCTACTATGCCGACTCCGTGAA
GGGCAGGTTCACAATCTCTCGCGATAACGCCAAGAATAGCCTGTATCTGC
AGATGAATTCCCTGAGGGCCGAGGACACAGCCGTGTACTATTGTGCCAGA
GGCTACGGCAAGGCCTATGATCAGTGGGGCCAGGGCACCCTGGTGACAGT
GTCTAGCCACCACCACCACCACCAC

SEQ ID NO. 6

ATGGAGACCGATACACTGCTGCTGTGGGTGCTGCTGCTGTGGGTGCCCGG
CAGCACCGGCAGCTCCGAGCTGACACAGGACCCCGCCGTGTCCGTGGCCC
TGGGACAGACCGTGAGGATCACATGCCAGGGCGACAGCCTGCGCTCCTAC
TATGCCAGCTGGTATCAGCAGAAGCCAGGACAGGCACCCGTGCTGGTCAT
CTACGGCAAGAACAATAGGCCTTCTGGCATCCCAGATCGCTTCAGCGGCT
CTAGCTCCGGCAACACCGCCTCTCTGACCATCACAGGAGCACAGGCAGAG
GACGAGGCAGATTACTATTGTAACTCCAGGGACTCTAGCGGCAATCCCCC
TGTGGTGTTTGGAGGAGGCACCAAGCTGACAGTGCTGGGCAGCCGCGGCG
GAGGAGGCTCTGGAGGAGGAGGCAGCGGCGGCGGCGGCTCCCTGGAGATG
GCCCAGGTGCAGCTGGTGGAGTCCGGAGGAGGACTGGTGCACCCAGGAGG
CTCTCTGAGGCTGAGCTGCGCAGCCTCCGGCTTCACCTTTCGGTCCCACT
CTATGAACTGGGTGAGACAGGCACCAGGCAAGGGCCTGGAGTGGGTGTCC
TCTATCAGCTCCGACTCCACCTACACATACTATGCCGATTCTGTGAAGGG
CCGGTTCACCATCTCCAGAGACAACGCCAAGAATTCTCTGTATCTGCAGA
TGAATAGCCTGCGGGCCGAGGATACAGCCGTGTACTATTGTGCCAGAAGC
GGCGGCCAGTGGAAGTACTATGACTACTGGGGCCAGGGCACCCTGGTGAC
AGTGTCTAGCCACCACCACCACCACCAC

SEQ ID NO. 7

ATGGAGACCGACACACTGCTGCTGTGGGTGCTGCTGCTGTGGGTGCCCGG
CTCCACCGGACAGTCTGTGGTGACACAGCCACCTAGCATGTCCGCCGCAC
CTGGACAGCAGGTGACCATCTCTTGCAGCGGCGGCAACTCCAATATCGAG
AGGAACTACGTGTCTTGGTATCTGCAGCTGCCAGGCACAGCCCCCAAGCT
GGTCATCTTCGACAATGATCGGAGACCTAGCGGCATCCCAGACCGCTTTT
CCGGCTCTAAGAGCGGCACCTCCGCCACACTGGGAATCACCGGACTGCAG
ACAGGCGACGAGGCAGATTACTATTGCGGCACCTGGGATAGCTCCCTGAG
GGGATGGGTGTTCGGAGGAGGCACCAAGCTGACAGTGCTGGGCTCCCGCG
GCGGAGGAGGCTCTGGAGGAGGAGGCAGCGGCGGCGGCGGCTCCCTGGAG
ATGGCCGAGGTGCAGCTGGTGGAGTCCGGAGGAGGACTGATCCAGCCAGG
AGGCAGCCTGAGGCTGTCCTGTGCAGCCTCTGGCTTCACCTTTAGCAACT
ACGCAATGAATTGGGTGCGGCAGGCACCAGGCAAGGGCCTGGAGTGGGTG
TCTACCATCAACGGCAGAGGCTCTAGCACAATCTATGCCGACAGCGTGAA
GGGCCGGTTTACCATCAGCAGAGATAACTCCAAGAATACACTGTACCTGC
AGATGAATAGCCTGAGAGCCGAGGACACCGCCACATACTATTGTGCCAGG
TATATCTCTCGCGGCCTGGGCGATAGCTGGGGACAGGGCACCCTGGTGAC
AGTGTCCTCTCACCACCACCACCACCAC

SEQ ID NO. 8

ATGGAGACCGACACACTGCTGCTGTGGGTGCTGCTGCTGTGGGTGCCCGG
CAGCACAGGACAGTCCGTGCTGACCCAGCCAGCCTCCGTGTCTGGCAGCC
CAGGCCAGTCTCTGACCATCAGCTGCACCGGCACATCTAACGATGTGGGC
GCCTACAAGTATGTGAGCTGGTATCAGCAGTATCCCGGCAAGGCCCCTAA
GCTGATCCTGTACGACGTGTTCAAGAGGCCTTCCGGCGTGTCTAACCGCT
TTTCCGGCTCTAAGAGCGATAATACAGCCTCCCTGACCATCTCTGGACTG
CAGGCAGAGGACGAGGCAGATTACTATTGCTTCAGCCTGACAAGCTCCAA
CACCTACGTGTTTGGCACCGGCACAAAGGTGACCGTGCTGGGCTCCCGGG
GCGGAGGAGGCAGCGGAGGAGGAGGCTCCGGCGGCGGCGGCTCTCTGGAG
ATGGCACAGATGCAGCTGGTGCAGAGCGGAGCAGAGGTGAAGAAGCCTGG
AGCCAGCGTGAAGGTGTCCTGTAAGGCCTCTGGCTACACATTCAATCGGT
ATGCCATCACCTGGGTGAGACAGGCACCAGGACAGGGCCTGGAGTGGATG
GGCTGGATCAGCGCCTACAACGGCAATTCCCACTATGCCCAGAAGCTGCA
GGGCCGGGTGACAATGACCACAGACACCTCCACAGGCACCGCCTACATGG
AGCTGCGGAGACTGAGGTCTGACGATACCGCCGTGTACTATTGTGCCCGC
ATGGCCTATGATTCCTGGGGCCAGGGCACACTGGTGACCGTGTCTAGCGG
CGGCGGCGGCAGCGGCGGCGGCGGATCCATGCTGCAGATGGCCGGCCAGT
GCAGCCAGAACGAGTACTTCGACAGCCTGCTGCACGCCTGCATCCCCTGC
CAGCTGAGGTGCAGCAGCAACACCCCCCCCCTGACCTGCCAGAGGTACTG
CAACGCCAGCGTGACCAACAGCGTGAAGGGCACCAACGCCCACCACCACC
ACCACCAC

SEQ ID NO. 9

ATGGAGACCGACACACTGCTGCTGTGGGTGCTGCTGCTGTGGGTGCCCGG
CTCTACCGGACAGAGCGTGCTGACACAGCCACCTTCCGCCTCTGGCACCC
CTGGACAGCGGGTGACAATCAGCTGCTCCGGCAGCCGGAGCAACGTGGGA
GGCAATTACGTGTTCTGGTATCAGCAGGTGCCAGGAGCAACCCCAAAGCT
GCTGATCTACCGGTCCAACCAGAGACCTTCTGGCGTGCCAGATCGGTTTG
CAGGCTCCAAGTCTGGCAGCTCCGCCTCCCTGGCCATCTCTGGACTGAGA
AGCGAGGACGAGGCCGATTACTATTGCGCCACCTGGGACGATAGCCTGTC
CGGCTTCGTGTTTGGCACCGGCACAAAGGTGACAGTGCTGGGCAGCCGGG
GCGGAGGAGGCAGCGGAGGAGGAGGCTCCGGCGGCGGCGGCTCTCTGGAG
ATGGCCGAGGTGCAGCTGGTGGAGTCCGGAGGAGGACTGGTGAAGCCAGG
AGGCTCCCTGAGGCTGTCTTGTGCAGCCAGCGGCTTCACCTTTAGCGACT
ACTATATGTCCTGGATCAGACAGGCACCAGGCAAGGGCCTGGAGTGGGTG
TCTTACATCTCTAGCTCCGGCAGCACCATCTACTATGCCGACTCCGTGAA
GGGCAGGTTCACAATCTCTCGCGATAACGCCAAGAATAGCCTGTATCTGC
AGATGAATTCCCTGAGGGCCGAGGACACAGCCGTGTACTATTGTGCCAGA
GGCTACGGCAAGGCCTATGATCAGTGGGGCCAGGGCACCCTGGTGACAGT
GTCTAGCGGCGGCGGCGGCAGCGGCGGATCCATGCTGCAGATGGCCGGCC
AGTGCAGCCAGAACGAGTACTTCGACAGCCTGCTGCACGCCTGCATCCCC
TGCCAGCTGAGGTGCAGCAGCAACACCCCCCCCCTGACCTGCCAGAGGTA
CTGCAACGCCAGCGTGACCAACAGCGTGAAGGGCACCAACGCCCACCACC
ACCACCACCACTGA

SEQ ID NO. 10

ATGGAGACCGATACACTGCTGCTGTGGGTGCTGCTGCTGTGGGTGCCCGG
CAGCACCGGCAGCTCCGAGCTGACACAGGACCCCGCCGTGTCCGTGGCCC
TGGGACAGACCGTGAGGATCACATGCCAGGGCGACAGCCTGCGCTCCTAC
TATGCCAGCTGGTATCAGCAGAAGCCAGGACAGGCACCCGTGCTGGTCAT
CTACGGCAAGAACAATAGGCCTTCTGGCATCCCAGATCGCTTCAGCGGCT
CTAGCTCCGGCAACACCGCCTCTCTGACCATCACAGGAGCACAGGCAGAG
GACGAGGCAGATTACTATTGTAACTCCAGGGACTCTAGCGGCAATCCCCC
TGTGGTGTTTGGAGGAGGCACCAAGCTGACAGTGCTGGGCAGCCGCGGCG
GAGGAGGCTCTGGAGGAGGAGGCAGCGGCGGCGGCGGCTCCCTGGAGATG
GCCCAGGTGCAGCTGGTGGAGTCCGGAGGAGGACTGGTGCACCCAGGAGG
CTCTCTGAGGCTGAGCTGCGCAGCCTCCGGCTTCACCTTTCGGTCCCACT
CTATGAACTGGGTGAGACAGGCACCAGGCAAGGGCCTGGAGTGGGTGTCC
TCTATCAGCTCCGACTCCACCTACACATACTATGCCGATTCTGTGAAGGG
CCGGTTCACCATCTCCAGAGACAACGCCAAGAATTCTCTGTATCTGCAGA
TGAATAGCCTGCGGGCCGAGGATACAGCCGTGTACTATTGTGCCAGAAGC
GGCGGCCAGTGGAAGTACTATGACTACTGGGGCCAGGGCACCCTGGTGAC
AGTGTCTAGCGGCGGCGGCGGCAGCGGCGGCGGCGGCAGCGGCGGCGGCG
GATCCATGCTGCAGATGGCCGGCCAGTGCAGCCAGAACGAGTACTTCGAC
AGCCTGCTGCACGCCTGCATCCCCTGCCAGCTGAGGTGCAGCAGCAACAC
CCCCCCCCTGACCTGCCAGAGGTACTGCAACGCCAGCGTGACCAACAGCG
TGAAGGGCACCAACGCCCACCACCACCACCACCAC

SEQ ID NO. 11

ATGGAGACCGACACACTGCTGCTGTGGGTGCTGCTGCTGTGGGTGCCCGG
CTCCACCGGACAGTCTGTGGTGACACAGCCACCTAGCATGTCCGCCGCAC
CTGGACAGCAGGTGACCATCTCTTGCAGCGGCGGCAACTCCAATATCGAG
AGGAACTACGTGTCTTGGTATCTGCAGCTGCCAGGCACAGCCCCCAAGCT
GGTCATCTTCGACAATGATCGGAGACCTAGCGGCATCCCAGACCGCTTTT
CCGGCTCTAAGAGCGGCACCTCCGCCACACTGGGAATCACCGGACTGCAG
ACAGGCGACGAGGCAGATTACTATTGCGGCACCTGGGATAGCTCCCTGAG
GGGATGGGTGTTCGGAGGAGGCACCAAGCTGACAGTGCTGGGCTCCCGCG
GCGGAGGAGGCTCTGGAGGAGGAGGCAGCGGCGGCGGCGGCTCCCTGGAG
ATGGCCGAGGTGCAGCTGGTGGAGTCCGGAGGAGGACTGATCCAGCCAGG
AGGCAGCCTGAGGCTGTCCTGTGCAGCCTCTGGCTTCACCTTTAGCAACT
ACGCAATGAATTGGGTGCGGCAGGCACCAGGCAAGGGCCTGGAGTGGGTG
TCTACCATCAACGGCAGAGGCTCTAGCACAATCTATGCCGACAGCGTGAA
GGGCCGGTTTACCATCAGCAGAGATAACTCCAAGAATACACTGTACCTGC
AGATGAATAGCCTGAGAGCCGAGGACACCGCCACATACTATTGTGCCAGG
TATATCTCTCGCGGCCTGGGCGATAGCTGGGGACAGGGCACCCTGGTGAC
AGTGTCCTCTGGCGGCGGCGGCAGCGGCGGCGGCGGCAGCGGCGGCGGCG
GATCCATGCTGCAGATGGCCGGCCAGTGCAGCCAGAACGAGTACTTCGAC
AGCCTGCTGCACGCCTGCATCCCCTGCCAGCTGAGGTGCAGCAGCAACAC
CCCCCCCCTGACCTGCCAGAGGTACTGCAACGCCAGCGTGACCAACAGCG
TGAAGGGCACCAACGCCCACCACCACCACCACCAC

SEQ ID NO. 12

METDTLLLWVLLLWVPGSTGDIVLTQSPPSLAMSLGKRATISCRASESVT
ILGSHLIHWYQQKPGQPPTLLIQLASNVQTGVPARFSGSGSRTDFTLTID
PVEEDDVAVYYCLQSRTIPRTFGGGTKLEIKGSTSGSGKPGSGEGSTKGQ
IQLVQSGPELKKPGETVKISCKASGYTFTDYSINWVKRAPGKGLKWMGWI
NTETREPAYAYDFRGRFAFSLETSASTAYLQINNLKYEDTATYFCALDYS
YAMDYWGQGTSVTVSSDYKDDDDKIEVMYPPPYLDNEKSNGTIIHVKGKH
LCPSPLFPGPSKPFWVLVVVGGVLACYSLLVTVAFIIFWVRSKRSRLLHS
DYMNMTPRRPGPTRKHYQPYAPPRDFAAYRSKRGRKKLLYIFKQPFMRPV
QTTQEEDGCSCRFPEEEEGGCELRVKFSRSADAPAYQQGQNQLYNELNLG
RREEYDVLDKRRGRDPEMGGKPQRRKNPQEGLYNELQKDKMAEAYSEIGM
KGERRRGKGHDGLYQGLSTATKDTYDALHMQALPPR

SEQ ID NO. 13

MRLLVLLWGCLLLPGYEAMLQMAGQCSQNEYFDSLLHACIPCQLRCSSNT
PPLTCQRYCNASVTNSVKGTNAGGGGSGGGGSGGGGSGGGGSDIQMTQSP
SSLSASVGDRVTITCRASQGIRSWLAWYQQKPEKAPKSLIYAASSLQSGV
PSRFSGSGSGTDFTLTISSLQPEDFATYYCQQYNSYPLTFGGGTKVEIKG
GGGSGGGGSGGGGSGGGGSQVQLVQSGAEVKKPGSSVKVSCKAFGGTFSS
YAISWVRQAPGQGLEWMGRIIRFLGIANYAQKFQGRVTLIADKSTNTAYM
ELSSLRSEDTAVYYCAGEPGERDPDAVDIWGQGTMVTVSSHHHHHH

SEQ ID NO. 14

MRLLVLLWGCLLLPGYEADIQMTQSPSSLSASVGDRVTITCRASQGIRSW
LAWYQQKPEKAPKSLIYAASSLQSGVPSRFSGSGSGTDFTLTISSLQPED
FATYYCQQYNSYPLTFGGGTKVEIKGGGGSGGGGSGGGGSGGGGSQVQLV
QSGAEVKKPGSSVKVSCKAFGGTFSSYAISWVRQAPGQGLEWMGRIIRFL
GIANYAQKFQGRVTLIADKSTNTAYMELSSLRSEDTAVYYCAGEPGERDP
DAVDIWGQGTMVTVSSGGGGSGGGGSGGGGSGGGGSMLQMAGQCSQNEYF
DSLLHACIPCQLRCSSNTPPLTCQRYCNASVTNSVKGTNAHHHHHH

SEQ ID NO. 15

METDTLLLWVLLLWVPGSTGQSVLTQPASVSGSPGQSLTISCTGTSNDVG
AYKYVSWYQQYPGKAPKLILYDVFKRPSGVSNRFSGSKSDNTASLTISGL
QAEDEADYYCFSLTSSNTYVFGTGTKVTVLGSRGGGGSGGGGSGGGGSLE
MAQMQLVQSGAEVKKPGASVKVSCKASGYTFNRYAITWVRQAPGQGLEWM
GWISAYNGNSHYAQKLQGRVTMTTDTSTGTAYMELRRLRSDDTAVYYCAR
MAYDSWGQGTLVTVSSHHHHHH

SEQ ID NO. 16

METDTLLLWVLLLWVPGSTGQSVLTQPPSASGTPGQRVTISCSGSRSNVG
GNYVFWYQQVPGATPKLLIYRSNQRPSGVPDRFAGSKSGSSASLAISGLR
SEDEADYYCATWDDSLSGFVFGTGTKVTVLGSRGGGGSGGGGSGGGGSLE
MAEVQLVESGGGLVKPGGSLRLSCAASGFTFSDYYMSWIRQAPGKGLEWV
SYISSSGSTIYYADSVKGRFTISRDNAKNSLYLQMNSLRAEDTAVYYCAR
GYGKAYDQWGQGTLVTVSSHHHHHH

SEQ ID NO. 17

METDTLLLWVLLLWVPGSTGSSELTQDPAVSVALGQTVRITCQGDSLRSY
YASWYQQKPGQAPVLVIYGKNNRPSGIPDRFSGSSSGNTASLTITGAQAE
DEADYYCNSRDSSGNPPVVFGGGTKLTVLGSRGGGGSGGGGSGGGGSLEM
AQVQLVESGGGLVHPGGSLRLSCAASGFTFRSHSMNWVRQAPGKGLEWVS
SISSDSTYTYYADSVKGRFTISRDNAKNSLYLQMNSLRAEDTAVYYCARS
GGQWKYYDYWGQGTLVTVSSHHHHHH

SEQ ID NO. 18

METDTLLLWVLLLWVPGSTGQSVVTQPPSMSAAPGQQVTISCSGGNSNIE
RNYVSWYLQLPGTAPKLVIFDNDRRPSGIPDRFSGSKSGTSATLGITGLQ
TGDEADYYCGTWDSSLRGWVFGGGTKLTVLGSRGGGGSGGGGSGGGGSLE
MAEVQLVESGGGLIQPGGSLRLSCAASGFTFSNYAMNWVRQAPGKGLEWV
STINGRGSSTIYADSVKGRFTISRDNSKNTLYLQMNSLRAEDTATYYCAR
YISRGLGDSWGQGTLVTVSSHHHHHH

SEQ ID NO. 19

METDTLLLWVLLLWVPGSTGQSVLTQPASVSGSPGQSLTISCTGTSNDVG
AYKYVSWYQQYPGKAPKLILYDVFKRPSGVSNRFSGSKSDNTASLTISGL
QAEDEADYYCFSLTSSNTYVFGTGTKVTVLGSRGGGGSGGGGSGGGGSLE
MAQMQLVQSGAEVKKPGASVKVSCKASGYTFNRYAITWVRQAPGQGLEWM
GWISAYNGNSHYAQKLQGRVTMTTDTSTGTAYMELRRLRSDDTAVYYCAR
MAYDSWGQGTLVTVSSGGGGSGGGGSMLQMAGQCSQNEYFDSLLHACIPC
QLRCSSNTPPLTCQRYCNASVTNSVKGTNAHHHHHH

SEQ ID NO. 20

METDTLLLWVLLLWVPGSTGQSVLTQPPSASGTPGQRVTISCSGSRSNVG
GNYVFWYQQVPGATPKLLIYRSNQRPSGVPDRFAGSKSGSSASLAISGLR
SEDEADYYCATWDDSLSGFVFGTGTKVTVLGSRGGGGSGGGGSGGGGSLE
MAEVQLVESGGGLVKPGGSLRLSCAASGFTFSDYYMSWIRQAPGKGLEWV
SYISSSGSTIYYADSVKGRFTISRDNAKNSLYLQMNSLRAEDTAVYYCAR
GYGKAYDQWGQGTLVTVSSGGGGSGGSMLQMAGQCSQNEYFDSLLHACIP
CQLRCSSNTPPLTCQRYCNASVTNSVKGTNAHHHHHH

SEQ ID NO. 21

METDTLLLWVLLLWVPGSTGSSELTQDPAVSVALGQTVRITCQGDSLRSY
YASWYQQKPGQAPVLVIYGKNNRPSGIPDRFSGSSSGNTASLTITGAQAE
DEADYYCNSRDSSGNPPVVFGGGTKLTVLGSRGGGGSGGGGSGGGGSLEM
AQVQLVESGGGLVHPGGSLRLSCAASGFTFRSHSMNWVRQAPGKGLEWVS
SISSDSTYTYYADSVKGRFTISRDNAKNSLYLQMNSLRAEDTAVYYCARS
GGQWKYYDYWGQGTLVTVSSGGGGSGGGGSGGGGSMLQMAGQCSQNEYFD
SLLHACIPCQLRCSSNTPPLTCQRYCNASVTNSVKGTNAHHHHHH

SEQ ID NO. 22

METDTLLLWVLLLWVPGSTGQSVVTQPPSMSAAPGQQVTISCSGGNSNIE
RNYVSWYLQLPGTAPKLVIFDNDRRPSGIPDRFSGSKSGTSATLGITGLQ
TGDEADYYCGTWDSSLRGWVFGGGTKLTVLGSRGGGGSGGGGSGGGGSLE
MAEVQLVESGGGLIQPGGSLRLSCAASGFTFSNYAMNWVRQAPGKGLEWV
STINGRGSSTIYADSVKGRFTISRDNSKNTLYLQMNSLRAEDTATYYCAR
YISRGLGDSWGQGTLVTVSSGGGGSGGGGSGGGGSMLQMAGQCSQNEYFD
SLLHACIPCQLRCSSNTPPLTCQRYCNASVTNSVKGTNAHHHHHH

SEQ ID NO. 23

MLQMAGQCSQNEYFDSLLHACIPCQLRCSSNTPPLTCQRYCNASVTNSVK
GTNA

SEQ ID NO. 24

GGGGS

SEQ ID NO. 25: A1 VL-028 VH scFv-His amino acid

MRLLVLLWGCLLLPGYEADIQMTQSPSSLSASVGDRVTITCRASQGISNY
LAWFQQKPGKAPKSLIYAASSLQSGVPSRFSGSGSGTDFTLTISSLQPED
FATYYCQQYNSYPITFGQGTRLEIKGGGGSGGGGSGGGGSGGGGSQVQLV
QSGAEVKKPGSSVKVSCKAFGGTFSSYAISWVRQAPGQGLEWMGRIIRFL
GIANYAQKFQGRVTLIADKSTNTAYMELSSLRSEDTAVYYCAGEPGERDP
DAVDIWGQGTMVTVSSHHHHHH

SEQ ID NO. 26:A1 VL-028 VH scFv-His nucleotide

ATGAGGCTGCTGGTGCTGCTGTGGGGCTGCCTGCTGCTGCCCGGCTACGA
GG CCGACATCCAGATGACCCAGAGCCCCAGCAGCCTGAGCGCCAGCGTG
GGCGA CAGGGTGACCATCACCTGCAGGGCCAGCCAGGGCATCAGCAACT
ACCTGGCC TGGTTCCAGCAGAAGCCCGGCAAGGCCCCCAAGAGCCTGAT
CTACGCCGCCA GCAGCCTGCAGAGCGGCGTGCCCAGCAGGTTCAGCGGC
AGCGGCAGCGGCAC CGACTTCACCCTGACCATCAGCAGCCTGCAGCCCG
AGGACTTCGCCACCTAC TACTGCCAGCAGTACAACAGCTACCCCATCAC
CTTCGGCCAGGGCACCAGGC TGGAGATCAAGGGCGGCGGCGGCAGCGGC
GGCGGCGGCAGCGGCGGCGGCGG CAGCGGCGGCGGCGGCAGCCAGGTGC
AGCTGGTGCAGAGCGGCGCCGAGGTG AAGAAGCCCGGCAGCAGCGTGAA
GGTGAGCTGCAAGGCCTTCGGCGGCACCT TCAGCAGCTACGCCATCAGC
TGGGTGAGGCAGGCCCCCGGCCAGGGCCTGGA GTGGATGGGCAGGATCA
TCAGGTTCCTGGGCATCGCCAACTACGCCCAGAAG TTCCAGGGCAGGGT
GACCCTGATCGCCGACAAGAGCACCAACACCGCCTACA TGGAGCTGAGC
AGCCTGAGGAGCGAGGACACCGCCGTGTACTACTGCGCCGG CGAGCCCG
GCGAGAGGGACCCCGACGCCGTGGACATCTGGGGCCAGGGCACC ATGGT
GACCGTGAGCAGCCACCACCACCACCACCAC

SEQ ID NO. 27:A3 VL-028 VH scFv-His amino acid

MRLLVLLWGCLLLPGYEADIQMTQSPSSLSASVGDRVTITCRASQSISSY
LN WYQQKPGKAPKLLIYAASSLQSGVPSRFSGSGSGTDFTLTISSLQPE
DFATY YCQQSYSTPLTFGGGTKVEIKGGGGSGGGGSGGGGSGGGGSQVQ
LVQSGAEV KKPGSSVKVSCKAFGGTFSSYAISWVRQAPGQGLEWMGRII
RFLGIANYAQK FQGRVTLIADKSTNTAYMELSSLRSEDTAVYYCAGEPG
ERDPDAVDIWGQGT MVTVSSHHHHHH

SEQ ID NO. 28:A3 VL-028 VH scFv-His nucleotide

ATGAGGCTGCTGGTGCTGCTGTGGGGCTGCCTGCTGCTGCCCGGCTACGA
GG CCGACATCCAGATGACCCAGAGCCCCAGCAGCCTGAGCGCCAGCGTG
GGCGA CAGGGTGACCATCACCTGCAGGGCCAGCCAGAGCATCAGCAGCT
ACCTGAAC TGGTACCAGCAGAAGCCCGGCAAGGCCCCCAAGCTGCTGAT
CTACGCCGCCA GCAGCCTGCAGAGCGGCGTGCCCAGCAGGTTCAGCGGC
AGCGGCAGCGGCAC CGACTTCACCCTGACCATCAGCAGCCTGCAGCCCG
AGGACTTCGCCACCTAC TACTGCCAGCAGAGCTACAGCACCCCCCTGAC
CTTCGGCGGCGGCACCAAGG TGGAGATCAAGGGCGGCGGCGGCAGCGGC
GGCGGCGGCAGCGGCGGCGGCGG CAGCGGCGGCGGCGGCAGCCAGGTGC
AGCTGGTGCAGAGCGGCGCCGAGGTG AAGAAGCCCGGCAGCAGCGTGAA
GGTGAGCTGCAAGGCCTTCGGCGGCACCT TCAGCAGCTACGCCATCAGC
TGGGTGAGGCAGGCCCCCGGCCAGGGCCTGGA GTGGATGGGCAGGATCA
TCAGGTTCCTGGGCATCGCCAACTACGCCCAGAAG TTCCAGGGCAGGGT
GACCCTGATCGCCGACAAGAGCACCAACACCGCCTACA TGGAGCTGAGC
AGCCTGAGGAGCGAGGACACCGCCGTGTACTACTGCGCCGG CGAGCCCG
GCGAGAGGGACCCCGACGCCGTGGACATCTGGGGCCAGGGCACC ATGGT
GACCGTGAGCAGCCACCACCACCACCACCAC

SEQ ID NO. 29:B1 VL-028 VH scFv-His amino acid

MRLLVLLWGCLLLPGYEAEIVLTQSPDFQSVTPKEKVTITCRASQSIGSS
LH WYQQKPDQSPKLLIKYASQSFSGVPSRFSGSGSGTDFTLTINSLEAE
DAATY YCHQSSSLPYTFGQGTKLEIKGGGGSGGGGSGGGGSGGGGSQVQ
LVQSGAEV KKPGSSVKVSCKAFGGTFSSYAISWVRQAPGQGLEWMGRII
RFLGIANYAQK FQGRVTLIADKSTNTAYMELSSLRSEDTAVYYCAGEPG
ERDPDAVDIWGQGT MVTVSSHHHHHH

SEQ ID NO. 30:B1 VL-028 VH scFv-His nucleotide

ATGAGGCTGCTGGTGCTGCTGTGGGGCTGCCTGCTGCTGCCCGGCTACGA
GG CCGAGATCGTGCTGACCCAGAGCCCCGACTTCCAGAGCGTGACCCCC
AAGGA GAAGGTGACCATCACCTGCAGGGCCAGCCAGAGCATCGGCAGCA
GCCTGCAC TGGTACCAGCAGAAGCCCGACCAGAGCCCCAAGCTGCTGAT
CAAGTACGCCA GCCAGAGCTTCAGCGGCGTGCCCAGCAGGTTCAGCGGC
AGCGGCAGCGGCAC CGACTTCACCCTGACCATCAACAGCCTGGAGGCCG
AGGACGCCGCCACCTAC TACTGCCACCAGAGCAGCAGCCTGCCCTACAC
CTTCGGCCAGGGCACCAAGC TGGAGATCAAGGGCGGCGGCGGCAGCGGC
GGCGGCGGCAGCGGCGGCGGCGG CAGCGGCGGCGGCGGCAGCCAGGTGC
AGCTGGTGCAGAGCGGCGCCGAGGTG AAGAAGCCCGGCAGCAGCGTGAA
GGTGAGCTGCAAGGCCTTCGGCGGCACCT TCAGCAGCTACGCCATCAGC
TGGGTGAGGCAGGCCCCCGGCCAGGGCCTGGA GTGGATGGGCAGGATCA
TCAGGTTCCTGGGCATCGCCAACTACGCCCAGAAG TTCCAGGGCAGGGT
GACCCTGATCGCCGACAAGAGCACCAACACCGCCTACA TGGAGCTGAGC
AGCCTGAGGAGCGAGGACACCGCCGTGTACTACTGCGCCGG CGAGCCCG
GCGAGAGGGACCCCGACGCCGTGGACATCTGGGGCCAGGGCACC ATGGT
GACCGTGAGCAGCCACCACCACCACCACCAC

SEQ ID NO. 31:B3 VL-028 VH scFv-His amino acid

MRLLVLLWGCLLLPGYEAAIQLTQSPSSLSASVGDRVTITCRASQGISSA
LA WYQQKPGKAPKLLIYDASSLESGVPSRFSGSGSGTDFTLTISSLQPE
DFATY YCQQFNSYPLTFGGGTKVEIKGGGGSGGGGSGGGGSGGGGSQVQ
LVQSGAEV KKPGSSVKVSCKAFGGTFSSYAISWVRQAPGQGLEWMGRII
RFLGIANYAQK FQGRVTLIADKSTNTAYMELSSLRSEDTAVYYCAGEPG
ERDPDAVDIWGQGT MVTVSSHHHHHH

SEQ ID NO. 32:B3 VL-028 VH scFv-His nucleotide

ATGAGGCTGCTGGTGCTGCTGTGGGGCTGCCTGCTGCTGCCCGGCTACGA
GG CCGCCATCCAGCTGACCCAGAGCCCCAGCAGCCTGAGCGCCAGCGTG
GGCGA CAGGGTGACCATCACCTGCAGGGCCAGCCAGGGCATCAGCAGCG
CCCTGGCC TGGTACCAGCAGAAGCCCGGCAAGGCCCCCAAGCTGCTGAT
CTACGACGCCA GCAGCCTGGAGAGCGGCGTGCCCAGCAGGTTCAGCGGC
AGCGGCAGCGGCAC CGACTTCACCCTGACCATCAGCAGCCTGCAGCCCG
AGGACTTCGCCACCTAC TACTGCCAGCAGTTCAACAGCTACCCCTGACC
TTCGGCGGCGGCACCAAGGT GGAGATCAAGGGCGGCGGCGGCAGCGGCG
GCGGCGGCAGCGGCGGCGGCGGC AGCGGCGGCGGCGGCAGCCAGGTGCA
GCTGGTGCAGAGCGGCGCCGAGGTGA AGAAGCCCGGCAGCAGCGTGAAG
GTGAGCTGCAAGGCCTTCGGCGGCACCTT CAGCAGCTACGCCATCAGCT
GGGTGAGGCAGGCCCCCGGCCAGGGCCTGGAG TGGATGGGCAGGATCAT
CAGGTTCCTGGGCATCGCCAACTACGCCCAGAAGT TCCAGGGCAGGGTG
ACCCTGATCGCCGACAAGAGCACCAACACCGCCTACAT GGAGCTGAGCA
GCCTGAGGAGCGAGGACACCGCCGTGTACTACTGCGCCGGC GAGCCCGG
CGAGAGGGACCCCGACGCCGTGGACATCTGGGGCCAGGGCACCA TGGTG
ACCGTGAGCAGCCACCACCACCACCACCAC

SEQ ID NO. 33: Bivalent A1 VL-028 VH-A1 VL-028 VH scFv amino acid

MRLLVLLWGCLLLPGYEADIQMTQSPSSLSASVGDRVTITCRASQGISNY
LA WFQQKPGKAPKSLIYAASSLQSGVPSRFSGSGSGTDFTLTISSLQPE
DFATY YCQQYNSYPITFGQGTRLEIKGGGGSGGGGSGGGGSGGGGSQVQ
LVQSGAEV KKPGSSVKVSCKAFGGTFSSYAISWVRQAPGQGLEWMGRII
RFLGIANYAQK FQGRVTLIADKSTNTAYMELSSLRSEDTAVYYCAGEPG
ERDPDAVDIWGQGT MVTVSSGGGGSGGGGSGGGGSGGGGSDIQMTQSPS
SLSASVGDRVTITCRAS QGISNYLAWFQQKPGKAPKSLIYAASSLQSGV
PSRFSGSGSGTDFTLTISSL QPEDFATYYCQQYNSYPITFGQGTRLEIK
GGGGSGGGGSGGGGSGGGGSQVQ LVQSGAEVKKPGSSVKVSCKAFGGTF
SSYAISWVRQAPGQGLEWMGRIIRFL GIANYAQKFQGRVTLIADKSTNT
AYMELSSLRSEDTAVYYCAGEPGERDPDA VDIWGQGTMVTVSSHHHHHH

SEQ ID NO. 34: Bivalent A1 VL-028 VH-A1 VL-028 VH scFv nucleotide

ATGAGGCTGCTGGTGCTGCTGTGGGGCTGCCTGCTGCTGCCCGGCTACGA
GG CCGACATCCAGATGACCCAGAGCCCCAGCAGCCTGAGCGCCAGCGTG
GGCGA CAGGGTGACCATCACCTGCAGGGCCAGCCAGGGCATCAGCAACT
ACCTGGCC TGGTTCCAGCAGAAGCCCGGCAAGGCCCCCAAGAGCCTGAT
CTACGCCGCCA GCAGCCTGCAGAGCGGCGTGCCCAGCAGGTTCAGCGGC
AGCGGCAGCGGCAC CGACTTCACCCTGACCATCAGCAGCCTGCAGCCCG
AGGACTTCGCCACCTAC TACTGCCAGCAGTACAACAGCTACCCCATCAC
CTTCGGCCAGGGCACCAGGC TGGAGATCAAGGGCGGCGGCGGCAGCGGC
GGCGGCGGCAGCGGCGGCGGCGG CAGCGGCGGCGGCGGCAGCCAGGTGC
AGCTGGTGCAGAGCGGCGCCGAGGTG AAGAAGCCCGGCAGCAGCGTGAA
GGTGAGCTGCAAGGCCTTCGGCGGCACCT TCAGCAGCTACGCCATCAGC
TGGGTGAGGCAGGCCCCCGGCCAGGGCCTGGA GTGGATGGGCAGGATCA
TCAGGTTCCTGGGCATCGCCAACTACGCCCAGAAG TTCCAGGGCAGGGT
GACCCTGATCGCCGACAAGAGCACCAACACCGCCTACA TGGAGCTGAGC
AGCCTGAGGAGCGAGGACACCGCCGTGTACTACTGCGCCGG CGAGCCCG
GCGAGAGGGACCCCGACGCCGTGGACATCTGGGGCCAGGGCACC ATGGT
GACCGTGAGCAGCGGCGGAGGCGGATCCGGCGGCGGCGGCAGCGGTG GC
GGAGGCTCCGGCGGAGGAGGCAGCGACATCCAGATGACCCAGAGCCCCAG
CAGCCTGAGCGCCAGCGTGGGCGACAGGGTGACCATCACCTGCAGGGCC
AGC CAGGGCATCAGCAACTACCTGGCCTGGTTCCAGCAGAAGCCCGGCA
AGGCCC CCAAGAGCCTGATCTACGCCGCCAGCAGCCTGCAGAGCGGCGT
GCCCAGCAG GTTCAGCGGCAGCGGCAGCGGCACCGACTTCACCCTGACC
ATCAGCAGCCTG CAGCCCGAGGACTTCGCCACCTACTACTGCCAGCAGT
ACAACAGCTACCCCA TCACCTTCGGCCAGGGCACCAGGCTGGAGATCAA
GGGCGGCGGCGGCAGCGG CGGCGGCGGCAGCGGCGGCGGCGGCAGCGGC
GGCGGCGGCAGCCAGGTGCAG CTGGTGCAGAGCGGCGCCGAGGTGAAGA
AGCCCGGCAGCAGCGTGAAGGTGA GCTGCAAGGCCTTCGGCGGCACCTT
CAGCAGCTACGCCATCAGCTGGGTGAG GCAGGCCCCCGGCCAGGGCCTG
GAGTGGATGGGCAGGATCATCAGGTTCCTG GGCATCGCCAACTACGCCC
AGAAGTTCCAGGGCAGGGTGACCCTGATCGCCG ACAAGAGCACCAACAC
CGCCTACATGGAGCTGAGCAGCCTGAGGAGCGAGGA CACCGCCGTGTAC
TACTGCGCCGGCGAGCCCGGCGAGAGGGACCCCGACGCC GTGGACATCT
GGGGCCAGGGCACCATGGTGACCGTGAGCAGCCACCACCACC ACCACCA
C

SEQ ID NO. 35: Bivalent A3 VL-028 VH-A3 VL-028 VH scFv amino acid

MRLLVLLWGCLLLPGYEADIQMTQSPSSLSASVGDRVTITCRASQSISSY
LNW YQQKPGKAPKLLIYAASSLQSGVPSRFSGSGSGTDFTLTISSLQPE
DFATYYC QQSYSTPLTFGGGTKVEIKGGGGSGGGGSGGGGSGGGGSQVQ
LVQSGAEVKKP GSSVKVSCKAFGGTFSSYAISWVRQAPGQGLEWMGRII
RFLGIANYAQKFQGR VTLIADKSTNTAYMELSSLRSEDTAVYYCAGEPG
ERDPDAVDIWGQGTMVTVS SGGGGSGGGGSGGGGSGGGGSDIQMTQSPS
SLSASVGDRVTITCRASQSISSY LNWYQQKPGKAPKLLIYAASSLQSGV
PSRFSGSGSGTDFTLTISSLQPEDFAT YYCQQSYSTPLTFGGGTKVEIK
GGGGSGGGGSGGGGSGGGGSQVQLVQSGAEV KKPGSSVKVSCKAFGGTF
SSYAISWVRQAPGQGLEWMGRIIRFLGIANYAQKF QGRVTLIADKSTNT
AYMELSSLRSEDTAVYYCAGEPGERDPDAVDIWGQGTMV TVSSHHHHHH

SEQ ID NO. 36: Bivalent A3 VL-028 VH-A3 VL-028 VH scFv nucleotide

ATGAGGCTGCTGGTGCTGCTGTGGGGCTGCCTGCTGCTGCCCGGCTACGA
GG CCGACATCCAGATGACCCAGAGCCCCAGCAGCCTGAGCGCCAGCGTG
GGCGA CAGGGTGACCATCACCTGCAGGGCCAGCCAGAGCATCAGCAGCT
ACCTGAAC TGGTACCAGCAGAAGCCCGGCAAGGCCCCCAAGCTGCTGAT
CTACGCCGCCA GCAGCCTGCAGAGCGGCGTGCCCAGCAGGTTCAGCGGC
AGCGGCAGCGGCAC CGACTTCACCCTGACCATCAGCAGCCTGCAGCCCG
AGGACTTCGCCACCTAC TACTGCCAGCAGAGCTACAGCACCCCCCTGAC
CTTCGGCGGCGGCACCAAGG TGGAGATCAAGGGCGGCGGCGGCAGCGGC
GGCGGCGGCAGCGGCGGCGGCGG CAGCGGCGGCGGCGGCAGCCAGGTGC
AGCTGGTGCAGAGCGGCGCCGAGGTG AAGAAGCCCGGCAGCAGCGTGAA
GGTGAGCTGCAAGGCCTTCGGCGGCACCT TCAGCAGCTACGCCATCAGC
TGGGTGAGGCAGGCCCCCGGCCAGGGCCTGGA GTGGATGGGCAGGATCA
TCAGGTTCCTGGGCATCGCCAACTACGCCCAGAAG TTCCAGGGCAGGGT
GACCCTGATCGCCGACAAGAGCACCAACACCGCCTACA TGGAGCTGAGC
AGCCTGAGGAGCGAGGACACCGCCGTGTACTACTGCGCCGG CGAGCCCG
GCGAGAGGGACCCCGACGCCGTGGACATCTGGGGCCAGGGCACC ATGGT
GACCGTGAGCAGCGGCGGAGGCGGATCCGGCGGCGGCGGCAGCGGTG GC
GGAGGCTCCGGCGGAGGAGGCAGCGACATCCAGATGACCCAGAGCCCCAG
CAGCCTGAGCGCCAGCGTGGGCGACAGGGTGACCATCACCTGCAGGGCC
AGC CAGAGCATCAGCAGCTACCTGAACTGGTACCAGCAGAAGCCCGGCA
AGGCCC CCAAGCTGCTGATCTACGCCGCCAGCAGCCTGCAGAGCGGCGT
GCCCAGCAG GTTCAGCGGCAGCGGCAGCGGCACCGACTTCACCCTGACC
ATCAGCAGCCTG CAGCCCGAGGACTTCGCCACCTACTACTGCCAGCAGA
GCTACAGCACCCCCC TGACCTTCGGCGGCGGCACCAAGGTGGAGATCAA
GGGCGGCGGCGGCAGCGG CGGCGGCGGCAGCGGCGGCGGCGGCAGCGGC
GGCGGCGGCAGCCAGGTGCAG CTGGTGCAGAGCGGCGCCGAGGTGAAGA
AGCCCGGCAGCAGCGTGAAGGTGA GCTGCAAGGCCTTCGGCGGCACCTT
CAGCAGCTACGCCATCAGCTGGGTGAG GCAGGCCCCCGGCCAGGGCCTG
GAGTGGATGGGCAGGATCATCAGGTTCCTG GGCATCGCCAACTACGCCC
AGAAGTTCCAGGGCAGGGTGACCCTGATCGCCG ACAAGAGCACCAACAC
CGCCTACATGGAGCTGAGCAGCCTGAGGAGCGAGGA CACCGCCGTGTAC
TACTGCGCCGGCGAGCCCGGCGAGAGGGACCCCGACGCC GTGGACATCT
GGGGCCAGGGCACCATGGTGACCGTGAGCAGCCACCACCACC ACCACCA
C

SEQ ID NO. 37: Bivalent B1 VL-028 VH-B1 VL-028 VH scFv amino acid

MRLLVLLWGCLLLPGYEAEIVLTQSPDFQSVTPKEKVTITCRASQSIGSS
LH WYQQKPDQSPKLLIKYASQSFSGVPSRFSGSGSGTDFTLTINSLEAE
DAATY YCHQSSSLPYTFGQGTKLEIKGGGGSGGGGSGGGGSGGGGSQVQ
LVQSGAEV KKPGSSVKVSCKAFGGTFSSYAISWVRQAPGQGLEWMGRII
RFLGIANYAQK FQGRVTLIADKSTNTAYMELSSLRSEDTAVYYCAGEPG
ERDPDAVDIWGQGT MVTVSSGGGGSGGGGSGGGGSGGGGSEIVLTQSPD
FQSVTPKEKVTITCRAS QSIGSSLHWYQQKPDQSPKLLIKYASQSFSGV
PSRFSGSGSGTDFTLTINSL EAEDAATYYCHQSSSLPYTFGQGTKLEIK
GGGGSGGGGSGGGGSGGGGSQVQ LVQSGAEVKKPGSSVKVSCKAFGGTF
SSYAISWVRQAPGQGLEWMGRIIRFL GIANYAQKFQGRVTLIADKSTNT
AYMELSSLRSEDTAVYYCAGEPGERDPDA VDIWGQGTMVTVSSHHHHHH

SEQ ID NO. 38: Bivalent B1 VL-028 VH-B1 VL-028 VH scFv nucleotide

ATGAGGCTGCTGGTGCTGCTGTGGGGCTGCCTGCTGCTGCCCGGCTACGA
GG CCGAGATCGTGCTGACCCAGAGCCCCGACTTCCAGAGCGTGACCCCC
AAGGA GAAGGTGACCATCACCTGCAGGGCCAGCCAGAGCATCGGCAGCA
GCCTGCAC TGGTACCAGCAGAAGCCCGACCAGAGCCCCAAGCTGCTGAT
CAAGTACGCCA GCCAGAGCTTCAGCGGCGTGCCCAGCAGGTTCAGCGGC
AGCGGCAGCGGCAC CGACTTCACCCTGACCATCAACAGCCTGGAGGCCG
AGGACGCCGCCACCTAC TACTGCCACCAGAGCAGCAGCCTGCCCTACAC
CTTCGGCCAGGGCACCAAGC TGGAGATCAAGGGCGGCGGCGGCAGCGGC
GGCGGCGGCAGCGGCGGCGGCGG CAGCGGCGGCGGCGGCAGCCAGGTGC
AGCTGGTGCAGAGCGGCGCCGAGGTG AAGAAGCCCGGCAGCAGCGTGAA
GGTGAGCTGCAAGGCCTTCGGCGGCACCT TCAGCAGCTACGCCATCAGC
TGGGTGAGGCAGGCCCCCGGCCAGGGCCTGGA GTGGATGGGCAGGATCA
TCAGGTTCCTGGGCATCGCCAACTACGCCCAGAAG TTCCAGGGCAGGGT
GACCCTGATCGCCGACAAGAGCACCAACACCGCCTACA TGGAGCTGAGC
AGCCTGAGGAGCGAGGACACCGCCGTGTACTACTGCGCCGG CGAGCCCG
GCGAGAGGGACCCCGACGCCGTGGACATCTGGGGCCAGGGCACC ATGGT
GACCGTGAGCAGCGGCGGAGGCGGATCCGGCGGCGGCGGCAGCGGTG GC
GGAGGCTCCGGCGGAGGAGGCAGCGAGATCGTGCTGACCCAGAGCCCCGA
CTTCCAGAGCGTGACCCCCAAGGAGAAGGTGACCATCACCTGCAGGGCC
AGC CAGAGCATCGGCAGCAGCCTGCACTGGTACCAGCAGAAGCCCGACC
AGAGCC CCAAGCTGCTGATCAAGTACGCCAGCCAGAGCTTCAGCGGCGT
GCCCAGCAG GTTCAGCGGCAGCGGCAGCGGCACCGACTTCACCCTGACC
ATCAACAGCCTG GAGGCCGAGGACGCCGCCACCTACTACTGCCACCAGA
GCAGCAGCCTGCCCT ACACCTTCGGCCAGGGCACCAAGCTGGAGATCAA
GGGCGGCGGCGGCAGCGG CGGCGGCGGCAGCGGCGGCGGCGGCAGCGGC
GGCGGCGGCAGCCAGGTGCAG CTGGTGCAGAGCGGCGCCGAGGTGAAGA
AGCCCGGCAGCAGCGTGAAGGTGA GCTGCAAGGCCTTCGGCGGCACCTT
CAGCAGCTACGCCATCAGCTGGGTGAG GCAGGCCCCCGGCCAGGGCCTG
GAGTGGATGGGCAGGATCATCAGGTTCCTG GGCATCGCCAACTACGCCC
AGAAGTTCCAGGGCAGGGTGACCCTGATCGCCG ACAAGAGCACCAACAC
CGCCTACATGGAGCTGAGCAGCCTGAGGAGCGAGGA CACCGCCGTGTAC
TACTGCGCCGGCGAGCCCGGCGAGAGGGACCCCGACGCC GTGGACATCT
GGGGCCAGGGCACCATGGTGACCGTGAGCAGCCACCACCACC ACCACCA
C

SEQ ID NO. 39: Bivalent B3 VL-028 VH-B3 VL-028 VH scFv amino acid

MRLLVLLWGCLLLPGYEAAIQLTQSPSSLSASVGDRVTITCRASQGISSA
LA WYQQKPGKAPKLLIYDASSLESGVPSRFSGSGSGTDFTLTISSLQPE
DFATY YCQQFNSYPLTFGGGTKVEIKGGGGSGGGGSGGGGSGGGGSQVQ
LVQSGAEV KKPGSSVKVSCKAFGGTFSSYAISWVRQAPGQGLEWMGRII
RFLGIANYAQK FQGRVTLIADKSTNTAYMELSSLRSEDTAVYYCAGEPG
ERDPDAVDIWGQGT MVTVSSGGGGSGGGGSGGGGSGGGGSAIQLTQSPS
SLSASVGDRVTITCRAS QGISSALAWYQQKPGKAPKLLIYDASSLESGV
PSRFSGSGSGTDFTLTISSL QPEDFATYYCQQFNSYPLTFGGGTKVEIK
GGGGSGGGGSGGGGSGGGGSQVQ LVQSGAEVKKPGSSVKVSCKAFGGTF
SSYAISWVRQAPGQGLEWMGRIIRFL GIANYAQKFQGRVTLIADKSTNT
AYMELSSLRSEDTAVYYCAGEPGERDPDA VDIWGQGTMVTVSSHHHHHH

SEQ ID NO. 40: Bivalent B3 VL-028 VH-B3 VL-028 VH scFv nucleotide

ATGAGGCTGCTGGTGCTGCTGTGGGGCTGCCTGCTGCTGCCCGGCTACGA
GGC CGCCATCCAGCTGACCCAGAGCCCCAGCAGCCTGAGCGCCAGCGTG
GGCGACA GGGTGACCATCACCTGCAGGGCCAGCCAGGGCATCAGCAGCG
CCCTGGCCTGG TACCAGCAGAAGCCCGGCAAGGCCCCCAAGCTGCTGAT
CTACGACGCCAGCAG CCTGGAGAGCGGCGTGCCCAGCAGGTTCAGCGGC
AGCGGCAGCGGCACCGACT TCACCCTGACCATCAGCAGCCTGCAGCCCG
AGGACTTCGCCACCTACTACTGC CAGCAGTTCAACAGCTACCCCCTGAC
CTTCGGCGGCGGCACCAAGGTGGAGAT CAAGGGCGGCGGCGGCAGCGGC
GGCGGCGGCAGCGGCGGCGGCGGCAGCGGCG GCGGCGGCAGCCAGGTGC
AGCTGGTGCAGAGCGGCGCCGAGGTGAAGAAGCCC GGCAGCAGCGTGAA
GGTGAGCTGCAAGGCCTTCGGCGGCACCTTCAGCAGCTA CGCCATCAGC
TGGGTGAGGCAGGCCCCCGGCCAGGGCCTGGAGTGGATGGGCA GGATCA
TCAGGTTCCTGGGCATCGCCAACTACGCCCAGAAGTTCCAGGGCAGG GT
GACCCTGATCGCCGACAAGAGCACCAACACCGCCTACATGGAGCTGAGCA
G CCTGAGGAGCGAGGACACCGCCGTGTACTACTGCGCCGGCGAGCCCGG
CGAGA GGGACCCCGACGCCGTGGACATCTGGGGCCAGGGCACCATGGTG
ACCGTGAGC AGCGGCGGAGGCGGATCCGGCGGCGGCGGCAGCGGTGGCG
GAGGCTCCGGCGG AGGAGGCAGCGCCATCCAGCTGACCCAGAGCCCCAG
CAGCCTGAGCGCCAGCG TGGGCGACAGGGTGACCATCACCTGCAGGGCC
AGCCAGGGCATCAGCAGCGCC CTGGCCTGGTACCAGCAGAAGCCCGGCA
AGGCCCCCAAGCTGCTGATCTACGA CGCCAGCAGCCTGGAGAGCGGCGT
GCCCAGCAGGTTCAGCGGCAGCGGCAGCG GCACCGACTTCACCCTGACC
ATCAGCAGCCTGCAGCCCGAGGACTTCGCCACC TACTACTGCCAGCAGT
TCAACAGCTACCCCCTGACCTTCGGCGGCGGCACCAA GGTGGAGATCAA
GGGCGGCGGCGGCAGCGGCGGCGGCGGCAGCGGCGGCGGCG GCAGCGGC
GGCGGCGGCAGCCAGGTGCAGCTGGTGCAGAGCGGCGCCGAGGTG AAGA
AGCCCGGCAGCAGCGTGAAGGTGAGCTGCAAGGCCTTCGGCGGCACCTT
CAGCAGCTACGCCATCAGCTGGGTGAGGCAGGCCCCCGGCCAGGGCCTGG
AGT GGATGGGCAGGATCATCAGGTTCCTGGGCATCGCCAACTACGCCCA
GAAGTTC CAGGGCAGGGTGACCCTGATCGCCGACAAGAGCACCAACACC
GCCTACATGGA GCTGAGCAGCCTGAGGAGCGAGGACACCGCCGTGTACT
ACTGCGCCGGCGAGC CCGGCGAGAGGGACCCCGACGCCGTGGACATCTG
GGGCCAGGGCACCATGGTG ACCGTGAGCAGCCACCACCACCACCACCAC

SEQ ID NO. 41: Bivalent A1 VL-028 VH-A1 VL-028 VH scFv -BCMA ECD amino acid

MRLLVLLWGCLLLPGYEADIQMTQSPSSLSASVGDRVTITCRASQGISNY
LAW FQQKPGKAPKSLIYAASSLQSGVPSRFSGSGSGTDFTLTISSLQPE
DFATYYC QQYNSYPITFGQGTRLEIKGGGGSGGGGSGGGGSGGGGSQVQ
LVQSGAEVKKP GSSVKVSCKAFGGTFSSYAISWVRQAPGQGLEWMGRII
RFLGIANYAQKFQGR VTLIADKSTNTAYMELSSLRSEDTAVYYCAGEPG
ERDPDAVDIWGQGTMVTVS SGGGGSGGGGSGGGGSGGGGSDIQMTQSPS
SLSASVGDRVTITCRASQGISNY LAWFQQKPGKAPKSLIYAASSLQSGV
PSRFSGSGSGTDFTLTISSLQPEDFAT YYCQQYNSYPITFGQGTRLEIK
GGGGSGGGGSGGGGSGGGGSQVQLVQSGAEV KKPGSSVKVSCKAFGGTF
SSYAISWVRQAPGQGLEWMGRIIRFLGIANYAQKF QGRVTLIADKSTNT
AYMELSSLRSEDTAVYYCAGEPGERDPDAVDIWGQGTMV TVSSGGGGSG
GGGSGGGGSGGGGSMLQMAGQCSQNEYFDSLLHACIPCQLRCS SNTPPL
TCQRYCNASVTNSVKGTNAHHHHHH

SEQ ID NO. 42: Bivalent A1 VL-028 VH-A1 VL-028 VH scFv -BCMA ECD nucleotide

ATGAGGCTGCTGGTGCTGCTGTGGGGCTGCCTGCTGCTGCCCGGCTACGA
GGC CGACATCCAGATGACCCAGAGCCCCAGCAGCCTGAGCGCCAGCGTG
GGCGACA GGGTGACCATCACCTGCAGGGCCAGCCAGGGCATCAGCAACT
ACCTGGCCTGG TTCCAGCAGAAGCCCGGCAAGGCCCCCAAGAGCCTGAT
CTACGCCGCCAGCAG CCTGCAGAGCGGCGTGCCCAGCAGGTTCAGCGGC
AGCGGCAGCGGCACCGACT TCACCCTGACCATCAGCAGCCTGCAGCCCG
AGGACTTCGCCACCTACTACTGC CAGCAGTACAACAGCTACCCCATCAC
CTTCGGCCAGGGCACCAGGCTGGAGAT CAAGGGCGGCGGCGGCAGCGGC
GGCGGCGGCAGCGGCGGCGGCGGCAGCGGCG GCGGCGGCAGCCAGGTGC
AGCTGGTGCAGAGCGGCGCCGAGGTGAAGAAGCCC GGCAGCAGCGTGAA
GGTGAGCTGCAAGGCCTTCGGCGGCACCTTCAGCAGCTA CGCCATCAGC
TGGGTGAGGCAGGCCCCCGGCCAGGGCCTGGAGTGGATGGGCA GGATCA
TCAGGTTCCTGGGCATCGCCAACTACGCCCAGAAGTTCCAGGGCAGG GT
GACCCTGATCGCCGACAAGAGCACCAACACCGCCTACATGGAGCTGAGCA
G CCTGAGGAGCGAGGACACCGCCGTGTACTACTGCGCCGGCGAGCCCGG
CGAGA GGGACCCCGACGCCGTGGACATCTGGGGCCAGGGCACCATGGTG
ACCGTGAGC AGCGGCGGAGGCGGATCCGGCGGCGGCGGCAGCGGTGGCG
GAGGCTCCGGCGG AGGAGGCAGCGACATCCAGATGACCCAGAGCCCCAG
CAGCCTGAGCGCCAGCG TGGGCGACAGGGTGACCATCACCTGCAGGGCC
AGCCAGGGCATCAGCAACTAC CTGGCCTGGTTCCAGCAGAAGCCCGGCA
AGGCCCCCAAGAGCCTGATCTACGC CGCCAGCAGCCTGCAGAGCGGCGT
GCCCAGCAGGTTCAGCGGCAGCGGCAGCG GCACCGACTTCACCCTGACC
ATCAGCAGCCTGCAGCCCGAGGACTTCGCCACC TACTACTGCCAGCAGT
ACAACAGCTACCCCATCACCTTCGGCCAGGGCACCAG GCTGGAGATCAA
GGGCGGCGGCGGCAGCGGCGGCGGCGGCAGCGGCGGCGGCG GCAGCGGC
GGCGGCGGCAGCCAGGTGCAGCTGGTGCAGAGCGGCGCCGAGGTG AAGA
AGCCCGGCAGCAGCGTGAAGGTGAGCTGCAAGGCCTTCGGCGGCACCTT
CAGCAGCTACGCCATCAGCTGGGTGAGGCAGGCCCCCGGCCAGGGCCTGG
AGT GGATGGGCAGGATCATCAGGTTCCTGGGCATCGCCAACTACGCCCA
GAAGTTC CAGGGCAGGGTGACCCTGATCGCCGACAAGAGCACCAACACC
GCCTACATGGA GCTGAGCAGCCTGAGGAGCGAGGACACCGCCGTGTACT
ACTGCGCCGGCGAGC CCGGCGAGAGGGACCCCGACGCCGTGGACATCTG
GGGCCAGGGCACCATGGTG ACCGTGAGCAGCGGCGGCGGCGGCAGCGGC
GGCGGCGGCAGCGGCGGCGGCGG CAGCGGCGGCGGCGGATCCATGCTGC
AGATGGCCGGCCAGTGCAGCCAGAACG AGTACTTCGACAGCCTGCTGCA
CGCCTGCATCCCCTGCCAGCTGAGGTGCAGC AGCAACACCCCCCCCCTG
ACCTGCCAGAGGTACTGCAACGCCAGCGTGACCAA CAGCGTGAAGGGCA
CCAACGCCCACCACCACCACCACCAC

SEQ ID NO. 43: BCMA ECD- bivalent A1 VL-028 VH-A1 VL-028 VH scFv amino acid

MRLLVLLWGCLLLPGYEAMLQMAGQCSQNEYFDSLLHACIPCQLRCSSNT
PPL TCQRYCNASVTNSVKGTNAGGGGSGGGGSGGGGSGGGGSDIQMTQS
PSSLSAS VGDRVTITCRASQGISNYLAWFQQKPGKAPKSLIYAASSLQS
GVPSRFSGSGS GTDFTLTISSLQPEDFATYYCQQYNSYPITFGQGTRLE
IKGGGGSGGGGSGGG GSGGGGSQVQLVQSGAEVKKPGSSVKVSCKAFGG
TFSSYAISWVRQAPGQGLE WMGRIIRFLGIANYAQKFQGRVTLIADKST
NTAYMELSSLRSEDTAVYYCAGE PGERDPDAVDIWGQGTMVTVSSGGGG
SGGGGSGGGGSGGGGSDIQMTQSPSSL SASVGDRVTITCRASQGISNYL
AWFQQKPGKAPKSLIYAASSLQSGVPSRFSG SGSGTDFTLTISSLQPED
FATYYCQQYNSYPITFGQGTRLEIKGGGGSGGGGS GGGGSGGGGSQVQL
VQSGAEVKKPGSSVKVSCKAFGGTFSSYAISWVRQAPGQ GLEWMGRIIR
FLGIANYAQKFQGRVTLIADKSTNTAYMELSSLRSEDTAVYYC AGEPGE
RDPDAVDIWGQGTMVTVSSHHHHHH

SEQ ID NO. 44: BCMA ECD- bivalent A1 VL-028 VH-A1 VL-028 VH scFv nucleotide

ATGAGGCTGCTGGTGCTGCTGTGGGGCTGCCTGCTGCTGCCCGGCTACGA
GGC CATGCTGCAGATGGCCGGCCAGTGCAGCCAGAACGAGTACTTCGAC
AGCCTGC TGCACGCCTGCATCCCCTGCCAGCTGAGGTGCAGCAGCAACA
CCCCCCCCCTG ACCTGCCAGAGGTACTGCAACGCCAGCGTGACCAACAG
CGTGAAGGGCACCAA CGCCGGCGGAGGCGGATCCGGCGGCGGCGGCAGC
GGTGGCGGAGGCTCCGGCG GAGGAGGCAGCGACATCCAGATGACCCAGA
GCCCCAGCAGCCTGAGCGCCAGC GTGGGCGACAGGGTGACCATCACCTG
CAGGGCCAGCCAGGGCATCAGCAACTA CCTGGCCTGGTTCCAGCAGAAG
CCCGGCAAGGCCCCCAAGAGCCTGATCTACG CCGCCAGCAGCCTGCAGA
GCGGCGTGCCCAGCAGGTTCAGCGGCAGCGGCAGC GGCACCGACTTCAC
CCTGACCATCAGCAGCCTGCAGCCCGAGGACTTCGCCAC CTACTACTGC
CAGCAGTACAACAGCTACCCCATCACCTTCGGCCAGGGCACCA GGCTGG
AGATCAAGGGCGGCGGCGGCAGCGGCGGCGGCGGCAGCGGCGGCGGC GG
CAGCGGCGGCGGCGGCAGCCAGGTGCAGCTGGTGCAGAGCGGCGCCGAGG
T GAAGAAGCCCGGCAGCAGCGTGAAGGTGAGCTGCAAGGCCTTCGGCGG
CACCT TCAGCAGCTACGCCATCAGCTGGGTGAGGCAGGCCCCCGGCCAG
GGCCTGGAG TGGATGGGCAGGATCATCAGGTTCCTGGGCATCGCCAACT
ACGCCCAGAAGTT CCAGGGCAGGGTGACCCTGATCGCCGACAAGAGCAC
CAACACCGCCTACATGG AGCTGAGCAGCCTGAGGAGCGAGGACACCGCC
GTGTACTACTGCGCCGGCGAG CCCGGCGAGAGGGACCCCGACGCCGTGG
ACATCTGGGGCCAGGGCACCATGGT GACCGTGAGCAGCGGCGGAGGCGG
ATCCGGCGGCGGCGGCAGCGGTGGCGGAG GCTCCGGCGGAGGAGGCAGC
GACATCCAGATGACCCAGAGCCCCAGCAGCCTG AGCGCCAGCGTGGGCG
ACAGGGTGACCATCACCTGCAGGGCCAGCCAGGGCAT CAGCAACTACCT
GGCCTGGTTCCAGCAGAAGCCCGGCAAGGCCCCCAAGAGCC TGATCTAC
GCCGCCAGCAGCCTGCAGAGCGGCGTGCCCAGCAGGTTCAGCGGC AGCG
GCAGCGGCACCGACTTCACCCTGACCATCAGCAGCCTGCAGCCCGAGGA
CTTCGCCACCTACTACTGCCAGCAGTACAACAGCTACCCCATCACCTTCG
GCC AGGGCACCAGGCTGGAGATCAAGGGCGGCGGCGGCAGCGGCGGCGG
CGGCAGC GGCGGCGGCGGCAGCGGCGGCGGCGGCAGCCAGGTGCAGCTG
GTGCAGAGCGG CGCCGAGGTGAAGAAGCCCGGCAGCAGCGTGAAGGTGA
GCTGCAAGGCCTTCG GCGGCACCTTCAGCAGCTACGCCATCAGCTGGGT
GAGGCAGGCCCCCGGCCAG GGCCTGGAGTGGATGGGCAGGATCATCAGG
TTCCTGGGCATCGCCAACTACGC CCAGAAGTTCCAGGGCAGGGTGACCC
TGATCGCCGACAAGAGCACCAACACCG CCTACATGGAGCTGAGCAGCCT
GAGGAGCGAGGACACCGCCGTGTACTACTGC GCCGGCGAGCCCGGCGAG
AGGGACCCCGACGCCGTGGACATCTGGGGCCAGGG CACCATGGTGACCG
TGAGCAGCCACCACCACCACCACCAC

SEQ ID NO. 45: Bivalent A3 VL-028 VH-A3 VL-028 VH scFv -BCMA ECD amino acid

MRLLVLLWGCLLLPGYEADIQMTQSPSSLSASVGDRVTITCRASQSISSY
LNW YQQKPGKAPKLLIYAASSLQSGVPSRFSGSGSGTDFTLTISSLQPE
DFATYYC QQSYSTPLTFGGGTKVEIKGGGGSGGGGSGGGGSGGGGSQVQ
LVQSGAEVKKP GSSVKVSCKAFGGTFSSYAISWVRQAPGQGLEWMGRII
RFLGIANYAQKFQGR VTLIADKSTNTAYMELSSLRSEDTAVYYCAGEPG
ERDPDAVDIWGQGTMVTVS SGGGGSGGGGSGGGGSGGGGSDIQMTQSPS
SLSASVGDRVTITCRASQSISSY LNWYQQKPGKAPKLLIYAASSLQSGV
PSRFSGSGSGTDFTLTISSLQPEDFAT YYCQQSYSTPLTFGGGTKVEIK
GGGGSGGGGSGGGGSGGGGSQVQLVQSGAEV KKPGSSVKVSCKAFGGTF
SSYAISWVRQAPGQGLEWMGRIIRFLGIANYAQKF QGRVTLIADKSTNT
AYMELSSLRSEDTAVYYCAGEPGERDPDAVDIWGQGTMV TVSSGGGGSG
GGGSGGGGSGGGGSMLQMAGQCSQNEYFDSLLHACIPCQLRCS SNTPPL
TCQRYCNASVTNSVKGTNAHHHHHH

SEQ ID NO. 46: Bivalent A3 VL-028 VH-A3 VL-028 VH scFv -BCMA ECD nucleotide

ATGAGGCTGCTGGTGCTGCTGTGGGGCTGCCTGCTGCTGCCCGGCTACGA
GGC CGACATCCAGATGACCCAGAGCCCCAGCAGCCTGAGCGCCAGCGTG
GGCGACA GGGTGACCATCACCTGCAGGGCCAGCCAGAGCATCAGCAGCT
ACCTGAACTGG TACCAGCAGAAGCCCGGCAAGGCCCCCAAGCTGCTGAT
CTACGCCGCCAGCAG CCTGCAGAGCGGCGTGCCCAGCAGGTTCAGCGGC
AGCGGCAGCGGCACCGACT TCACCCTGACCATCAGCAGCCTGCAGCCCG
AGGACTTCGCCACCTACTACTGC CAGCAGAGCTACAGCACCCCCCTGAC
CTTCGGCGGCGGCACCAAGGTGGAGAT CAAGGGCGGCGGCGGCAGCGGC
GGCGGCGGCAGCGGCGGCGGCGGCAGCGGCG GCGGCGGCAGCCAGGTGC
AGCTGGTGCAGAGCGGCGCCGAGGTGAAGAAGCCC GGCAGCAGCGTGAA
GGTGAGCTGCAAGGCCTTCGGCGGCACCTTCAGCAGCTA CGCCATCAGC
TGGGTGAGGCAGGCCCCCGGCCAGGGCCTGGAGTGGATGGGCA GGATCA
TCAGGTTCCTGGGCATCGCCAACTACGCCCAGAAGTTCCAGGGCAGG GT
GACCCTGATCGCCGACAAGAGCACCAACACCGCCTACATGGAGCTGAGCA
G CCTGAGGAGCGAGGACACCGCCGTGTACTACTGCGCCGGCGAGCCCGG
CGAGA GGGACCCCGACGCCGTGGACATCTGGGGCCAGGGCACCATGGTG
ACCGTGAGC AGCGGCGGAGGCGGATCCGGCGGCGGCGGCAGCGGTGGCG
GAGGCTCCGGCGG AGGAGGCAGCGACATCCAGATGACCCAGAGCCCCAG
CAGCCTGAGCGCCAGCG TGGGCGACAGGGTGACCATCACCTGCAGGGCC
AGCCAGAGCATCAGCAGCTAC CTGAACTGGTACCAGCAGAAGCCCGGCA
AGGCCCCCAAGCTGCTGATCTACGC CGCCAGCAGCCTGCAGAGCGGCGT
GCCCAGCAGGTTCAGCGGCAGCGGCAGCG GCACCGACTTCACCCTGACC
ATCAGCAGCCTGCAGCCCGAGGACTTCGCCACC TACTACTGCCAGCAGA
GCTACAGCACCCCCCTGACCTTCGGCGGCGGCACCAA GGTGGAGATCAA
GGGCGGCGGCGGCAGCGGCGGCGGCGGCAGCGGCGGCGGCG GCAGCGGC
GGCGGCGGCAGCCAGGTGCAGCTGGTGCAGAGCGGCGCCGAGGTG AAGA
AGCCCGGCAGCAGCGTGAAGGTGAGCTGCAAGGCCTTCGGCGGCACCTT
CAGCAGCTACGCCATCAGCTGGGTGAGGCAGGCCCCCGGCCAGGGCCTGG
AGT GGATGGGCAGGATCATCAGGTTCCTGGGCATCGCCAACTACGCCCA
GAAGTTC CAGGGCAGGGTGACCCTGATCGCCGACAAGAGCACCAACACC
GCCTACATGGA GCTGAGCAGCCTGAGGAGCGAGGACACCGCCGTGTACT
ACTGCGCCGGCGAGC CCGGCGAGAGGGACCCCGACGCCGTGGACATCTG
GGGCCAGGGCACCATGGTG ACCGTGAGCAGCGGCGGCGGCGGCAGCGGC
GGCGGCGGCAGCGGCGGCGGCGG CAGCGGCGGCGGCGGATCCATGCTGC
AGATGGCCGGCCAGTGCAGCCAGAACG AGTACTTCGACAGCCTGCTGCA
CGCCTGCATCCCCTGCCAGCTGAGGTGCAGC AGCAACACCCCCCCCCTG
ACCTGCCAGAGGTACTGCAACGCCAGCGTGACCAA CAGCGTGAAGGGCA
CCAACGCCCACCACCACCACCACCAC

SEQ ID NO. 47: BCMA ECD- bivalent A3 VL-028 VH-A3 VL-028 VH scFv amino acid

MRLLVLLWGCLLLPGYEAMLQMAGQCSQNEYFDSLLHACIPCQLRCSSNT
PPL TCQRYCNASVTNSVKGTNAGGGGSGGGGSGGGGSGGGGSDIQMTQS
PSSLSAS VGDRVTITCRASQSISSYLNWYQQKPGKAPKLLIYAASSLQS
GVPSRFSGSGS GTDFTLTISSLQPEDFATYYCQQSYSTPLTFGGGTKVE
IKGGGGSGGGGSGGG GSGGGGSQVQLVQSGAEVKKPGSSVKVSCKAFGG
TFSSYAISWVRQAPGQGLE WMGRIIRFLGIANYAQKFQGRVTLIADKST
NTAYMELSSLRSEDTAVYYCAGE PGERDPDAVDIWGQGTMVTVSSGGGG
SGGGGSGGGGSGGGGSDIQMTQSPSSL SASVGDRVTITCRASQSISSYL
NWYQQKPGKAPKLLIYAASSLQSGVPSRFSG SGSGTDFTLTISSLQPED
FATYYCQQSYSTPLTFGGGTKVEIKGGGGSGGGGS GGGGSGGGGSQVQL
VQSGAEVKKPGSSVKVSCKAFGGTFSSYAISWVRQAPGQ GLEWMGRIIR
FLGIANYAQKFQGRVTLIADKSTNTAYMELSSLRSEDTAVYYC AGEPGE
RDPDAVDIWGQGTMVTVSSHHHHHH

SEQ ID NO. 48: BCMA ECD- bivalent A3 VL-028 VH-A3 VL-028 VH scFv nucleotide

ATGAGGCTGCTGGTGCTGCTGTGGGGCTGCCTGCTGCTGCCCGGCTACGA
GGC CATGCTGCAGATGGCCGGCCAGTGCAGCCAGAACGAGTACTTCGAC
AGCCTGC TGCACGCCTGCATCCCCTGCCAGCTGAGGTGCAGCAGCAACA
CCCCCCCCCTG ACCTGCCAGAGGTACTGCAACGCCAGCGTGACCAACAG
CGTGAAGGGCACCAA CGCCGGCGGAGGCGGATCCGGCGGCGGCGGCAGC
GGTGGCGGAGGCTCCGGCG GAGGAGGCAGCGACATCCAGATGACCCAGA
GCCCCAGCAGCCTGAGCGCCAGC GTGGGCGACAGGGTGACCATCACCTG
CAGGGCCAGCCAGAGCATCAGCAGCTA CCTGAACTGGTACCAGCAGAAG
CCCGGCAAGGCCCCCAAGCTGCTGATCTACG CCGCCAGCAGCCTGCAGA
GCGGCGTGCCCAGCAGGTTCAGCGGCAGCGGCAGC GGCACCGACTTCAC
CCTGACCATCAGCAGCCTGCAGCCCGAGGACTTCGCCAC CTACTACTGC
CAGCAGAGCTACAGCACCCCCCTGACCTTCGGCGGCGGCACCA AGGTGG
AGATCAAGGGCGGCGGCGGCAGCGGCGGCGGCGGCAGCGGCGGCGGC GG
CAGCGGCGGCGGCGGCAGCCAGGTGCAGCTGGTGCAGAGCGGCGCCGAGG
T GAAGAAGCCCGGCAGCAGCGTGAAGGTGAGCTGCAAGGCCTTCGGCGG
CACCT TCAGCAGCTACGCCATCAGCTGGGTGAGGCAGGCCCCCGGCCAG
GGCCTGGAG TGGATGGGCAGGATCATCAGGTTCCTGGGCATCGCCAACT
ACGCCCAGAAGTT CCAGGGCAGGGTGACCCTGATCGCCGACAAGAGCAC
CAACACCGCCTACATGG AGCTGAGCAGCCTGAGGAGCGAGGACACCGCC
GTGTACTACTGCGCCGGCGAG CCCGGCGAGAGGGACCCCGACGCCGTGG
ACATCTGGGGCCAGGGCACCATGGT GACCGTGAGCAGCGGCGGAGGCGG
ATCCGGCGGCGGCGGCAGCGGTGGCGGAG GCTCCGGCGGAGGAGGCAGC
GACATCCAGATGACCCAGAGCCCCAGCAGCCTG AGCGCCAGCGTGGGCG
ACAGGGTGACCATCACCTGCAGGGCCAGCCAGAGCAT CAGCAGCTACCT
GAACTGGTACCAGCAGAAGCCCGGCAAGGCCCCCAAGCTGC TGATCTAC
GCCGCCAGCAGCCTGCAGAGCGGCGTGCCCAGCAGGTTCAGCGGC AGCG
GCAGCGGCACCGACTTCACCCTGACCATCAGCAGCCTGCAGCCCGAGGA
CTTCGCCACCTACTACTGCCAGCAGAGCTACAGCACCCCCCTGACCTTCG
GCG GCGGCACCAAGGTGGAGATCAAGGGCGGCGGCGGCAGCGGCGGCGG
CGGCAGC GGCGGCGGCGGCAGCGGCGGCGGCGGCAGCCAGGTGCAGCTG
GTGCAGAGCGG CGCCGAGGTGAAGAAGCCCGGCAGCAGCGTGAAGGTGA
GCTGCAAGGCCTTCG GCGGCACCTTCAGCAGCTACGCCATCAGCTGGGT
GAGGCAGGCCCCCGGCCAG GGCCTGGAGTGGATGGGCAGGATCATCAGG
TTCCTGGGCATCGCCAACTACGC CCAGAAGTTCCAGGGCAGGGTGACCC
TGATCGCCGACAAGAGCACCAACACCG CCTACATGGAGCTGAGCAGCCT
GAGGAGCGAGGACACCGCCGTGTACTACTGC GCCGGCGAGCCCGGCGAG
AGGGACCCCGACGCCGTGGACATCTGGGGCCAGGG CACCATGGTGACCG
TGAGCAGCCACCACCACCACCACCAC

SEQ ID NO. 49: Bivalent B1 VL-028 VH-B1 VL-028 VH scFv -BCMA ECD amino acid

MRLLVLLWGCLLLPGYEAEIVLTQSPDFQSVTPKEKVTITCRASQSIGSS
LHW YQQKPDQSPKLLIKYASQSFSGVPSRFSGSGSGTDFTLTINSLEAE
DAATYYC HQSSSLPYTFGQGTKLEIKGGGGSGGGGSGGGGSGGGGSQVQ
LVQSGAEVKKP GSSVKVSCKAFGGTFSSYAISWVRQAPGQGLEWMGRII
RFLGIANYAQKFQGR VTLIADKSTNTAYMELSSLRSEDTAVYYCAGEPG
ERDPDAVDIWGQGTMVTVS SGGGGSGGGGSGGGGSGGGGSEIVLTQSPD
FQSVTPKEKVTITCRASQSIGSS LHWYQQKPDQSPKLLIKYASQSFSGV
PSRFSGSGSGTDFTLTINSLEAEDAAT YYCHQSSSLPYTFGQGTKLEIK
GGGGSGGGGSGGGGSGGGGSQVQLVQSGAEV KKPGSSVKVSCKAFGGTF
SSYAISWVRQAPGQGLEWMGRIIRFLGIANYAQKF QGRVTLIADKSTNT
AYMELSSLRSEDTAVYYCAGEPGERDPDAVDIWGQGTMV TVSSGGGGSG
GGGSGGGGSGGGGSMLQMAGQCSQNEYFDSLLHACIPCQLRCS SNTPPL
TCQRYCNASVTNSVKGTNAHHHHHH

SEQ ID NO. 50: Bivalent B1 VL-028 VH-B1 VL-028 VH scFv -BCMA ECD nucleotide

ATGAGGCTGCTGGTGCTGCTGTGGGGCTGCCTGCTGCTGCCCGGCTACGA
GGC CGAGATCGTGCTGACCCAGAGCCCCGACTTCCAGAGCGTGACCCCC
AAGGAGA AGGTGACCATCACCTGCAGGGCCAGCCAGAGCATCGGCAGCA
GCCTGCACTGG TACCAGCAGAAGCCCGACCAGAGCCCCAAGCTGCTGAT
CAAGTACGCCAGCCA GAGCTTCAGCGGCGTGCCCAGCAGGTTCAGCGGC
AGCGGCAGCGGCACCGACT TCACCCTGACCATCAACAGCCTGGAGGCCG
AGGACGCCGCCACCTACTACTGC CACCAGAGCAGCAGCCTGCCCTACAC
CTTCGGCCAGGGCACCAAGCTGGAGAT CAAGGGCGGCGGCGGCAGCGGC
GGCGGCGGCAGCGGCGGCGGCGGCAGCGGCG GCGGCGGCAGCCAGGTGC
AGCTGGTGCAGAGCGGCGCCGAGGTGAAGAAGCCC GGCAGCAGCGTGAA
GGTGAGCTGCAAGGCCTTCGGCGGCACCTTCAGCAGCTA CGCCATCAGC
TGGGTGAGGCAGGCCCCCGGCCAGGGCCTGGAGTGGATGGGCA GGATCA
TCAGGTTCCTGGGCATCGCCAACTACGCCCAGAAGTTCCAGGGCAGG GT
GACCCTGATCGCCGACAAGAGCACCAACACCGCCTACATGGAGCTGAGCA
G CCTGAGGAGCGAGGACACCGCCGTGTACTACTGCGCCGGCGAGCCCGG
CGAGA GGGACCCCGACGCCGTGGACATCTGGGGCCAGGGCACCATGGTG
ACCGTGAGC AGCGGCGGAGGCGGATCCGGCGGCGGCGGCAGCGGTGGCG
GAGGCTCCGGCGG AGGAGGCAGCGAGATCGTGCTGACCCAGAGCCCCGA
CTTCCAGAGCGTGACCC CCAAGGAGAAGGTGACCATCACCTGCAGGGCC
AGCCAGAGCATCGGCAGCAGC CTGCACTGGTACCAGCAGAAGCCCGACC
AGAGCCCCAAGCTGCTGATCAAGTA CGCCAGCCAGAGCTTCAGCGGCGT
GCCCAGCAGGTTCAGCGGCAGCGGCAGCG GCACCGACTTCACCCTGACC
ATCAACAGCCTGGAGGCCGAGGACGCCGCCACC TACTACTGCCACCAGA
GCAGCAGCCTGCCCTACACCTTCGGCCAGGGCACCAA GCTGGAGATCAA
GGGCGGCGGCGGCAGCGGCGGCGGCGGCAGCGGCGGCGGCG GCAGCGGC
GGCGGCGGCAGCCAGGTGCAGCTGGTGCAGAGCGGCGCCGAGGTG AAGA
AGCCCGGCAGCAGCGTGAAGGTGAGCTGCAAGGCCTTCGGCGGCACCTT
CAGCAGCTACGCCATCAGCTGGGTGAGGCAGGCCCCCGGCCAGGGCCTGG
AGT GGATGGGCAGGATCATCAGGTTCCTGGGCATCGCCAACTACGCCCA
GAAGTTC CAGGGCAGGGTGACCCTGATCGCCGACAAGAGCACCAACACC
GCCTACATGGA GCTGAGCAGCCTGAGGAGCGAGGACACCGCCGTGTACT
ACTGCGCCGGCGAGC CCGGCGAGAGGGACCCCGACGCCGTGGACATCTG
GGGCCAGGGCACCATGGTG ACCGTGAGCAGCGGCGGCGGCGGCAGCGGC
GGCGGCGGCAGCGGCGGCGGCGG CAGCGGCGGCGGCGGATCCATGCTGC
AGATGGCCGGCCAGTGCAGCCAGAACG AGTACTTCGACAGCCTGCTGCA
CGCCTGCATCCCCTGCCAGCTGAGGTGCAGC AGCAACACCCCCCCCCTG
ACCTGCCAGAGGTACTGCAACGCCAGCGTGACCAA CAGCGTGAAGGGCA
CCAACGCCCACCACCACCACCACCAC

SEQ ID NO. 51: BCMA ECD- bivalent B1 VL-028 VH-B1 VL-028 VH scFv amino acid

MRLLVLLWGCLLLPGYEAMLQMAGQCSQNEYFDSLLHACIPCQLRCSSNT
PPL TCQRYCNASVTNSVKGTNAGGGGSGGGGSGGGGSGGGGSEIVLTQS
PDFQSVT PKEKVTITCRASQSIGSSLHWYQQKPDQSPKLLIKYASQSFS
GVPSRFSGSGS GTDFTLTINSLEAEDAATYYCHQSSSLPYTFGQGTKLE
IKGGGGSGGGGSGGG GSGGGGSQVQLVQSGAEVKKPGSSVKVSCKAFGG
TFSSYAISWVRQAPGQGLE WMGRIIRFLGIANYAQKFQGRVTLIADKST
NTAYMELSSLRSEDTAVYYCAGE PGERDPDAVDIWGQGTMVTVSSGGGG
SGGGGSGGGGSGGGGSEIVLTQSPDFQ SVTPKEKVTITCRASQSIGSSL
HWYQQKPDQSPKLLIKYASQSFSGVPSRFSG SGSGTDFTLTINSLEAED
AATYYCHQSSSLPYTFGQGTKLEIKGGGGSGGGGS GGGGSGGGGSQVQL
VQSGAEVKKPGSSVKVSCKAFGGTFSSYAISWVRQAPGQ GLEWMGRIIR
FLGIANYAQKFQGRVTLIADKSTNTAYMELSSLRSEDTAVYYC AGEPGE
RDPDAVDIWGQGTMVTVSSHHHHHH

SEQ ID NO. 52: BCMA ECD- bivalent B1 VL-028 VH-B1 VL-028 VH scFv nucleotide

ATGAGGCTGCTGGTGCTGCTGTGGGGCTGCCTGCTGCTGCCCGGCTACGA
GGCC ATGCTGCAGATGGCCGGCCAGTGCAGCCAGAACGAGTACTTCGAC
AGCCTGCTG CACGCCTGCATCCCCTGCCAGCTGAGGTGCAGCAGCAACA
CCCCCCCCCTGACC TGCCAGAGGTACTGCAACGCCAGCGTGACCAACAG
CGTGAAGGGCACCAACGCC GGCGGAGGCGGATCCGGCGGCGGCGGCAGC
GGTGGCGGAGGCTCCGGCGGAGGA GGCAGCGAGATCGTGCTGACCCAGA
GCCCCGACTTCCAGAGCGTGACCCCCAAG GAGAAGGTGACCATCACCTG
CAGGGCCAGCCAGAGCATCGGCAGCAGCCTGCAC TGGTACCAGCAGAAG
CCCGACCAGAGCCCCAAGCTGCTGATCAAGTACGCCAGC CAGAGCTTCA
GCGGCGTGCCCAGCAGGTTCAGCGGCAGCGGCAGCGGCACCGAC TTCAC
CCTGACCATCAACAGCCTGGAGGCCGAGGACGCCGCCACCTACTACTGC
CACCAGAGCAGCAGCCTGCCCTACACCTTCGGCCAGGGCACCAAGCTGGA
GATC AAGGGCGGCGGCGGCAGCGGCGGCGGCGGCAGCGGCGGCGGCGGC
AGCGGCGGC GGCGGCAGCCAGGTGCAGCTGGTGCAGAGCGGCGCCGAGG
TGAAGAAGCCCGGC AGCAGCGTGAAGGTGAGCTGCAAGGCCTTCGGCGG
CACCTTCAGCAGCTACGCC ATCAGCTGGGTGAGGCAGGCCCCCGGCCAG
GGCCTGGAGTGGATGGGCAGGATC ATCAGGTTCCTGGGCATCGCCAACT
ACGCCCAGAAGTTCCAGGGCAGGGTGACC CTGATCGCCGACAAGAGCAC
CAACACCGCCTACATGGAGCTGAGCAGCCTGAGG AGCGAGGACACCGCC
GTGTACTACTGCGCCGGCGAGCCCGGCGAGAGGGACCCC GACGCCGTGG
ACATCTGGGGCCAGGGCACCATGGTGACCGTGAGCAGCGGCGGA GGCGG
ATCCGGCGGCGGCGGCAGCGGTGGCGGAGGCTCCGGCGGAGGAGGCAGC
GAGATCGTGCTGACCCAGAGCCCCGACTTCCAGAGCGTGACCCCCAAGGA
GAAG GTGACCATCACCTGCAGGGCCAGCCAGAGCATCGGCAGCAGCCTG
CACTGGTAC CAGCAGAAGCCCGACCAGAGCCCCAAGCTGCTGATCAAGT
ACGCCAGCCAGAGC TTCAGCGGCGTGCCCAGCAGGTTCAGCGGCAGCGG
CAGCGGCACCGACTTCACC CTGACCATCAACAGCCTGGAGGCCGAGGAC
GCCGCCACCTACTACTGCCACCAG AGCAGCAGCCTGCCCTACACCTTCG
GCCAGGGCACCAAGCTGGAGATCAAGGGC GGCGGCGGCAGCGGCGGCGG
CGGCAGCGGCGGCGGCGGCAGCGGCGGCGGCGGC AGCCAGGTGCAGCTG
GTGCAGAGCGGCGCCGAGGTGAAGAAGCCCGGCAGCAGC GTGAAGGTGA
GCTGCAAGGCCTTCGGCGGCACCTTCAGCAGCTACGCCATCAGC TGGGT
GAGGCAGGCCCCCGGCCAGGGCCTGGAGTGGATGGGCAGGATCATCAGG
TTCCTGGGCATCGCCAACTACGCCCAGAAGTTCCAGGGCAGGGTGACCCT
GATC GCCGACAAGAGCACCAACACCGCCTACATGGAGCTGAGCAGCCTG
AGGAGCGAG GACACCGCCGTGTACTACTGCGCCGGCGAGCCCGGCGAGA
GGGACCCCGACGCC GTGGACATCTGGGGCCAGGGCACCATGGTGACCGT
GAGCAGCCACCACCACCAC CACCAC

SEQ ID NO. 53: Bivalent B3 VL-028 VH-B3 VL-028 VH scFv -BCMA ECD amino acid

MRLLVLLWGCLLLPGYEAAIQLTQSPSSLSASVGDRVTITCRASQGISSA
LAWY QQKPGKAPKLLIYDASSLESGVPSRFSGSGSGTDFTLTISSLQPE
DFATYYCQQ FNSYPLTFGGGTKVEIKGGGGSGGGGSGGGGSGGGGSQVQ
LVQSGAEVKKPGSS VKVSCKAFGGTFSSYAISWVRQAPGQGLEWMGRII
RFLGIANYAQKFQGRVTLI ADKSTNTAYMELSSLRSEDTAVYYCAGEPG
ERDPDAVDIWGQGTMVTVSSGGGG SGGGGSGGGGSGGGGSAIQLTQSPS
SLSASVGDRVTITCRASQGISSALAWYQQ KPGKAPKLLIYDASSLESGV
PSRFSGSGSGTDFTLTISSLQPEDFATYYCQQFN SYPLTFGGGTKVEIK
GGGGSGGGGSGGGGSGGGGSQVQLVQSGAEVKKPGSSVK VSCKAFGGTF
SSYAISWVRQAPGQGLEWMGRIIRFLGIANYAQKFQGRVTLIAD KSTNT
AYMELSSLRSEDTAVYYCAGEPGERDPDAVDIWGQGTMVTVSSGGGGSG
GGGSGGGGSGGGGSMLQMAGQCSQNEYFDSLLHACIPCQLRCSSNTPPLT
CQRY CNASVTNSVKGTNAHHHHHH

SEQ ID NO. 54: Bivalent B3 VL-028 VH-B3 VL-028 VH scFv -BCMA ECD nucleotide

ATGAGGCTGCTGGTGCTGCTGTGGGGCTGCCTGCTGCTGCCCGGCTACGA
GGC CGCCATCCAGCTGACCCAGAGCCCCAGCAGCCTGAGCGCCAGCGTG
GGCGACA GGGTGACCATCACCTGCAGGGCCAGCCAGGGCATCAGCAGCG
CCCTGGCCTGG TACCAGCAGAAGCCCGGCAAGGCCCCCAAGCTGCTGAT
CTACGACGCCAGCAG CCTGGAGAGCGGCGTGCCCAGCAGGTTCAGCGGC
AGCGGCAGCGGCACCGACT TCACCCTGACCATCAGCAGCCTGCAGCCCG
AGGACTTCGCCACCTACTACTGC CAGCAGTTCAACAGCTACCCCCTGAC
CTTCGGCGGCGGCACCAAGGTGGAGAT CAAGGGCGGCGGCGGCAGCGGC
GGCGGCGGCAGCGGCGGCGGCGGCAGCGGCG GCGGCGGCAGCCAGGTGC
AGCTGGTGCAGAGCGGCGCCGAGGTGAAGAAGCCC GGCAGCAGCGTGAA
GGTGAGCTGCAAGGCCTTCGGCGGCACCTTCAGCAGCTA CGCCATCAGC
TGGGTGAGGCAGGCCCCCGGCCAGGGCCTGGAGTGGATGGGCA GGATCA
TCAGGTTCCTGGGCATCGCCAACTACGCCCAGAAGTTCCAGGGCAGG GT
GACCCTGATCGCCGACAAGAGCACCAACACCGCCTACATGGAGCTGAGCA
G CCTGAGGAGCGAGGACACCGCCGTGTACTACTGCGCCGGCGAGCCCGG
CGAGA GGGACCCCGACGCCGTGGACATCTGGGGCCAGGGCACCATGGTG
ACCGTGAGC AGCGGCGGAGGCGGATCCGGCGGCGGCGGCAGCGGTGGCG
GAGGCTCCGGCGG AGGAGGCAGCGCCATCCAGCTGACCCAGAGCCCCAG
CAGCCTGAGCGCCAGCG TGGGCGACAGGGTGACCATCACCTGCAGGGCC
AGCCAGGGCATCAGCAGCGCC CTGGCCTGGTACCAGCAGAAGCCCGGCA
AGGCCCCCAAGCTGCTGATCTACGA CGCCAGCAGCCTGGAGAGCGGCGT
GCCCAGCAGGTTCAGCGGCAGCGGCAGCG GCACCGACTTCACCCTGACC
ATCAGCAGCCTGCAGCCCGAGGACTTCGCCACC TACTACTGCCAGCAGT
TCAACAGCTACCCCCTGACCTTCGGCGGCGGCACCAA GGTGGAGATCAA
GGGCGGCGGCGGCAGCGGCGGCGGCGGCAGCGGCGGCGGCG GCAGCGGC
GGCGGCGGCAGCCAGGTGCAGCTGGTGCAGAGCGGCGCCGAGGTG AAGA
AGCCCGGCAGCAGCGTGAAGGTGAGCTGCAAGGCCTTCGGCGGCACCTT
CAGCAGCTACGCCATCAGCTGGGTGAGGCAGGCCCCCGGCCAGGGCCTGG
AGT GGATGGGCAGGATCATCAGGTTCCTGGGCATCGCCAACTACGCCCA
GAAGTTC CAGGGCAGGGTGACCCTGATCGCCGACAAGAGCACCAACACC
GCCTACATGGA GCTGAGCAGCCTGAGGAGCGAGGACACCGCCGTGTACT
ACTGCGCCGGCGAGC CCGGCGAGAGGGACCCCGACGCCGTGGACATCTG
GGGCCAGGGCACCATGGTG ACCGTGAGCAGCGGCGGCGGCGGCAGCGGC
GGCGGCGGCAGCGGCGGCGGCGG CAGCGGCGGCGGCGGATCCATGCTGC
AGATGGCCGGCCAGTGCAGCCAGAACG AGTACTTCGACAGCCTGCTGCA
CGCCTGCATCCCCTGCCAGCTGAGGTGCAGC AGCAACACCCCCCCCCTG
ACCTGCCAGAGGTACTGCAACGCCAGCGTGACCAA CAGCGTGAAGGGCA
CCAACGCCCACCACCACCACCACCAC

SEQ ID NO. 55: BCMA ECD- bivalent B3 VL-028 VH-B3 VL-028 VH scFv amino acid

MRLLVLLWGCLLLPGYEAMLQMAGQCSQNEYFDSLLHACIPCQLRCSSNT
PPL TCQRYCNASVTNSVKGTNAGGGGSGGGGSGGGGSGGGGSAIQLTQS
PSSLSAS VGDRVTITCRASQGISSALAWYQQKPGKAPKLLIYDASSLES
GVPSRFSGSGS GTDFTLTISSLQPEDFATYYCQQFNSYPLTFGGGTKVE
IKGGGGSGGGGSGGG GSGGGGSQVQLVQSGAEVKKPGSSVKVSCKAFGG
TFSSYAISWVRQAPGQGLE WMGRIIRFLGIANYAQKFQGRVTLIADKST
NTAYMELSSLRSEDTAVYYCAGE PGERDPDAVDIWGQGTMVTVSSGGGG
SGGGGSGGGGSGGGGSAIQLTQSPSSL SASVGDRVTITCRASQGISSAL
AWYQQKPGKAPKLLIYDASSLESGVPSRFSG SGSGTDFTLTISSLQPED
FATYYCQQFNSYPLTFGGGTKVEIKGGGGSGGGGS GGGGSGGGGSQVQL
VQSGAEVKKPGSSVKVSCKAFGGTFSSYAISWVRQAPGQ GLEWMGRIIR
FLGIANYAQKFQGRVTLIADKSTNTAYMELSSLRSEDTAVYYC AGEPGE
RDPDAVDIWGQGTMVTVSSHHHHHH

SEQ ID NO. 56: BCMA ECD- bivalent B3 VL-028 VH-B3 VL-028 VH scFv nucleotide

ATGAGGCTGCTGGTGCTGCTGTGGGGCTGCCTGCTGCTGCCCGGCTACGA
GGC CATGCTGCAGATGGCCGGCCAGTGCAGCCAGAACGAGTACTTCGAC
AGCCTGC TGCACGCCTGCATCCCCTGCCAGCTGAGGTGCAGCAGCAACA
CCCCCCCCCTG ACCTGCCAGAGGTACTGCAACGCCAGCGTGACCAACAG
CGTGAAGGGCACCAA CGCCGGCGGAGGCGGATCCGGCGGCGGCGGCAGC
GGTGGCGGAGGCTCCGGCG GAGGAGGCAGCGCCATCCAGCTGACCCAGA
GCCCCAGCAGCCTGAGCGCCAGC GTGGGCGACAGGGTGACCATCACCTG
CAGGGCCAGCCAGGGCATCAGCAGCGC CCTGGCCTGGTACCAGCAGAAG
CCCGGCAAGGCCCCCAAGCTGCTGATCTACG ACGCCAGCAGCCTGGAGA
GCGGCGTGCCCAGCAGGTTCAGCGGCAGCGGCAGC GGCACCGACTTCAC
CCTGACCATCAGCAGCCTGCAGCCCGAGGACTTCGCCAC CTACTACTGC
CAGCAGTTCAACAGCTACCCCCTGACCTTCGGCGGCGGCACCA AGGTGG
AGATCAAGGGCGGCGGCGGCAGCGGCGGCGGCGGCAGCGGCGGCGGC GG
CAGCGGCGGCGGCGGCAGCCAGGTGCAGCTGGTGCAGAGCGGCGCCGAGG
T GAAGAAGCCCGGCAGCAGCGTGAAGGTGAGCTGCAAGGCCTTCGGCGG
CACCT TCAGCAGCTACGCCATCAGCTGGGTGAGGCAGGCCCCCGGCCAG
GGCCTGGAG TGGATGGGCAGGATCATCAGGTTCCTGGGCATCGCCAACT
ACGCCCAGAAGTT CCAGGGCAGGGTGACCCTGATCGCCGACAAGAGCAC
CAACACCGCCTACATGG AGCTGAGCAGCCTGAGGAGCGAGGACACCGCC
GTGTACTACTGCGCCGGCGAG CCCGGCGAGAGGGACCCCGACGCCGTGG
ACATCTGGGGCCAGGGCACCATGGT GACCGTGAGCAGCGGCGGAGGCGG
ATCCGGCGGCGGCGGCAGCGGTGGCGGAG GCTCCGGCGGAGGAGGCAGC
GCCATCCAGCTGACCCAGAGCCCCAGCAGCCTG AGCGCCAGCGTGGGCG
ACAGGGTGACCATCACCTGCAGGGCCAGCCAGGGCAT CAGCAGCGCCCT
GGCCTGGTACCAGCAGAAGCCCGGCAAGGCCCCCAAGCTGC TGATCTAC
GACGCCAGCAGCCTGGAGAGCGGCGTGCCCAGCAGGTTCAGCGGC AGCG
GCAGCGGCACCGACTTCACCCTGACCATCAGCAGCCTGCAGCCCGAGGA
CTTCGCCACCTACTACTGCCAGCAGTTCAACAGCTACCCCCTGACCTTCG
GCG GCGGCACCAAGGTGGAGATCAAGGGCGGCGGCGGCAGCGGCGGCGG
CGGCAGC GGCGGCGGCGGCAGCGGCGGCGGCGGCAGCCAGGTGCAGCTG
GTGCAGAGCGG CGCCGAGGTGAAGAAGCCCGGCAGCAGCGTGAAGGTGA
GCTGCAAGGCCTTCG GCGGCACCTTCAGCAGCTACGCCATCAGCTGGGT
GAGGCAGGCCCCCGGCCAG GGCCTGGAGTGGATGGGCAGGATCATCAGG
TTCCTGGGCATCGCCAACTACGC CCAGAAGTTCCAGGGCAGGGTGACCC
TGATCGCCGACAAGAGCACCAACACCG CCTACATGGAGCTGAGCAGCCT
GAGGAGCGAGGACACCGCCGTGTACTACTGC GCCGGCGAGCCCGGCGAG
AGGGACCCCGACGCCGTGGACATCTGGGGCCAGGG CACCATGGTGACCG
TGAGCAGCCACCACCACCACCACCAC

SEQ ID NO. 57

QVQLVQSGAEVKKPGSSVKVSCKAFGGTFSSYAISWVRQAPGQGLEWMGR
IIR FLGIANYAQKFQGRVTLIADKSTNTAYMELSSLRSEDTAVYYCAGE
PGERDPD AVDIWGQGTMVTVSS.

SEQ ID NO. 58

DIQMTQSPSSLSASVGDRVTITCRASQGIRSWLAWYQQKPEKAPKSLIYA
ASS LQSGVPSRFSGSGSGTDFTLTISSLQPEDFATYYCQQYNSYPLTFG
GGTKVEI K

What is claimed is:

1. A method of treating a subject having or suffering from multiple myeloma, the method comprising:
administering to the subject a fusion protein comprising:
(a) an antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and
(b) a polypeptide antigen comprising a second multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the first multiple myeloma antigen and the second multiple myeloma antigen are different;
wherein the subject (i) previously received ACT (e.g., CAR-T cell therapy), that binds to a cancer cell expressing the second multiple myeloma antigen, (ii) previously exhibited at least one beneficial response to the ACT (e.g., CAR-T cell therapy), and (iii) prior to administration of the fusion protein, the subject exhibits at least one nonbeneficial response to the ACT (e.g., CAR-T cell therapy).

2. A method of treating a subject having or suffering from multiple myeloma, the method comprising:
administering to the subject a fusion protein comprising:
(a) an antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and CD138; and
(b) a BCMA polypeptide;
wherein the subject (i) previously received ACT that binds to a cancer cell expressing a BCMA polypeptide, (ii) previously exhibited at least one beneficial response to the ACT, and (iii) prior to administration of the fusion protein, the subject exhibits at least one nonbeneficial response to the ACT.

3. A method of treating a subject having or suffering from multiple myeloma, the method comprising:
administering to the subject a fusion protein comprising:
(a) an antigen binding polypeptide that binds a first multiple myeloma antigen selected from the group consisting of CD38; CS⅟SLAMF7; GPRC5D; CD208 (LAMP3); CD307e (FCRL5); ITGA8; ITGB7; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and CD138; and
(b) a BCMA polypeptide;
wherein the subject (i) previously received an anti-BCMA CAR-T cell, (ii) previously exhibited at least one beneficial response to the anti-BCMA CAR-T cell, and (iii) prior to administration of the fusion protein, the subject exhibits at least one nonbeneficial response to the anti-BCMA CAR-T cell.

4. The method of any one of claims 1-3, wherein the ACT comprises administering a cell selected from the group consisting of NK cells, tumor-infiltrating lymphocytes (TIL), autologous or allogeneic CAR-T cells, myeloid-derived cells, Induced pluripotent stem cells (IPSC), gamma delta T cells, invariant NK cells, and NK-T cells .

5. The method of claim 1, wherein the method further comprises measuring a level of expression of the second multiple myeloma antigen, e.g., in a sample from the subject (e.g., a biological sample, e.g., a tumor sample).

6. The method of claim 2 or 3, wherein the method further comprises measuring a level of expression of the BCMA polypeptide, e.g., in a sample from the subject (e.g., a biological sample, e.g., a tumor sample).

7. The method of any one of claims 1-6, wherein the beneficial response comprises clearance, regression, and/or or stabilization of the cancer, e.g., over a defined period of time (e.g., at least 1, 2, 3, 4, 5, 6, 7, 8, 9, 10, 11, 12 weeks, or at least 1, 2, 3, 4, 5, 6, 7, 8, 9, 10, 11, 12 months, or at least 1, 2, 3, 4, 5, 6, 7, 8, 9, 10, 11, 12 years).

8. The method of any one of claims 1-7, wherein the beneficial response comprises an absence of relapse, recurrence, and/or metastasis of the cancer, e.g., over a defined period of time (e.g., at least 1, 2, 3, 4, 5, 6, 7, 8, 9, 10, 11, 12 weeks, or at least 1, 2, 3, 4, 5, 6, 7, 8, 9, 10, 11, 12 months, or at least 1, 2, 3, 4, 5, 6, 7, 8, 9, 10, 11, 12 years).

9. The method of any one of claims 1-8, wherein the nonbeneficial response comprises a relapse, recurrence, and/or metastasis of the cancer.

10. The method of any one of claims 5, and 7-9, wherein prior to administration of the fusion protein, the measured level of expression of the second multiple myeloma antigen is reduced relative to a control level (e.g., a level of expression of the second multiple myeloma antigen in a subject exhibiting at least one beneficial response to the ACT; and/or the level of expression of the second multiple myeloma antigen in the subject during a period in which the subject previously exhibited a beneficial response to the ACT).

11. The method of any one of claims 6-9, wherein prior to administration of the fusion protein, the measured level of expression of the BCMA polypeptide is reduced relative to a control level (e.g., a level of expression of the BCMA polypeptide in a subject exhibiting at least one beneficial response to the ACT; and/or the level of expression of the BCMA polypeptide in the subject during a period in which the subject previously exhibited a beneficial response to the ACT).

12. The method of claims 10 or 11, wherein a reduced level of expression, relative to the control, results in the subject exhibiting at least one nonbeneficial response to the ACT.

13. The method of any one of claims 10-12, wherein the measured level of expression is about 90%, 80%, 70%, 60%, 50%, 40%, 30%, 20%, 10%, or less, of the control level.

14. The method of any one of claims 1-13, further comprising administering the ACT to the subject, e.g., within about 6 hours, 12 hours, 18 hours, 24 hours, 2, 3, 4, 5, 6, 7 days of administering the fusion protein.

15. The method of any one of claims 1-14, wherein after administration of the fusion protein, the subject exhibits at least one beneficial response to the ACT and/or a reduction in at least one nonbeneficial response to the ACT.

16. The method of any one of claims 1-14, wherein the fusion protein comprises 2 or more antigen binding polypeptides.

17. The method of claim 16, wherein the fusion protein comprises 2 antigen binding polypeptides.

18. The method of claim 17, wherein the fusion protein comprises:
(a) a first antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA;
(b) a second antigen binding polypeptide that binds a second multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and
(c) a polypeptide antigen comprising of a third multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the first, second, and third multiple myeloma antigens are different.

19. The method of claim 17, wherein the fusion protein comprises:
(a) a first antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA;
(b) a second antigen binding polypeptide that binds a second multiple myeloma antigen selected from the group consisting of CD38; CS⅟SLAMF7; GPRC5D; CD208 (LAMP3);1/ CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and
(c) a polypeptide antigen comprising of a third multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the third multiple myeloma antigen is different than the first and second.

20. The method of claim 19, wherein the first multiple myeloma antigen and the second multiple myeloma antigen are the same.

21. The method of claim 19, wherein the first multiple myeloma antigen and the second multiple myeloma antigen are CD38.

22. The method of claim 20 or 21, wherein the first and second antigen binding polypeptide are the same, and the fusion protein comprises two copies of the same antigen binding polypeptide.

23. The method of any one of claims 20-23, wherein the first and second antigen binding polypeptide bind to the first and second multiple myeloma antigen at a Kd of about 50 nM to about 2 µM.

24. The method of any one of claims 20-23, wherein the fusion protein binds to a tumor cell expressing the first and second multiple myeloma antigen (e.g., CD38) with higher avidity relative to a healthy or non-tumor cell.

25. The method of claim 24, wherein the fusion protein binds to the tumor cell at a Kd of about 1 to about 40 nM.

26. The method of claim 18, wherein the first multiple myeloma antigen is CD38 and the second multiple myeloma antigen is GPRC5D.

27. A method of treating a subject having or suffering from multiple myeloma, the method comprising administering to the subject:
(a) a fusion protein comprising (i) a polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and (ii) a second multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the first multiple myeloma antigen and the second multiple myeloma antigen are different; and
(b) ACT, wherein the ACT comprises a cell that binds to a cancer cell expressing the second multiple myeloma antigen.

28. A method of treating a subject having or suffering from multiple myeloma, the method comprising administering to the subject:
(a) a fusion protein comprising (i) a polypeptide that binds a first multiple myeloma antigen selected from the group consisting of CD38; CS1/SLAMF7; GPRC5D; CD208 (LAMP3); CD307e (FCRL5); ITGA8; ITGB7; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and CD138; and (ii) a BCMA polypeptide; and
(b) ACT (e.g., CAR-T cell therapy), wherein the ACT comprises a cell (e.g., a CAR-T cell) that binds to a cancer cell expressing the BCMA polypeptide.

29. A method of treating a subject having or suffering from multiple myeloma, the method comprising administering to the subject:
(a) a fusion protein comprising (i) a polypeptide that binds a first multiple myeloma antigen selected from the group consisting of CD38; CS1/SLAMF7; GPRC5D; CD208 (LAMP3); CD307e (FCRL5); ITGA8; ITGB7; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and CD138; and (ii) a BCMA polypeptide; and
(b) an anti-BCMA CAR-T cell.

30. The method of claim 27 or 28, wherein administration of the fusion protein and the ACT is more effective in treating multiple myeloma, relative to a control multiple myeloma subject receiving ACT alone.

31. The method of claim 30, wherein the control multiple myeloma subject exhibits at least one nonbeneficial response to the ACT.

32. The method of claim 29, wherein administration of the fusion protein and the anti-BCMA CAR-T cell is more effective in treating the multiple myeloma, relative to a control multiple myeloma subject receiving anti-BCMA CAR-T therapy alone.

33. The method of claim 32, wherein the control multiple myeloma subject exhibits at least one nonbeneficial response to the anti-BCMA CAR-T therapy.

34. The method of claim 30 or 31, wherein the ACT comprises administering a cell selected from the group consisting of NK cells, tumor-infiltrating lymphocytes (TIL), autologous or allogeneic CAR-T cells, myeloid-derived cells, Induced pluripotent stem cells (IPSC), gamma delta T cells, invariant NK cells, and NK-T cells .

35. The method of any one of claims 27-34, wherein the fusion protein comprises 2 or more polypeptides that bind a multiple myeloma antigen.

36. The method of claim 35, wherein the fusion protein comprises 2 polypeptides that bind a multiple myeloma antigen.

37. The method of claim 36, wherein the fusion protein comprises:
(i) a first polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA;
(ii) a second polypeptide that binds a second multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and
(iii) a third multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the first, second, and third multiple myeloma antigens are different.

38. The method of claim 36, wherein the fusion protein comprises:
(i) a first polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA;
(ii) a second polypeptide that binds a second multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and
(iii) a third multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the third multiple myeloma antigen is different than the first and second myeloma antigen.

39. The method of claim 38, wherein the first multiple myeloma antigen and the second multiple myeloma antigen are the same.

40. The method of claim 38, wherein the first multiple myeloma antigen and the second multiple myeloma antigen are CD38.

41. The method of claim 39 or 40, wherein the first and second antigen binding polypeptide are the same, and the fusion protein comprises two copies of the same antigen binding polypeptide.

42. The method of any one of claims 39-41, wherein the first and second antigen binding polypeptide bind to the first and second multiple myeloma antigen at a Kd of about 50 nM to about 2 µM.

43. The method of any one of claims 39-41, wherein the fusion protein binds to a tumor cell expressing the first and second multiple myeloma antigen (e.g., CD38) with higher avidity relative to a healthy or non-tumor cell.

44. The method of claim 43, wherein the fusion protein binds to the tumor cell at a Kd of about 1 to about 40 nM.

45. The method of claim 37, wherein the first multiple myeloma antigen is CD38 and the second multiple myeloma antigen is GPRC5D.

46. A method of treating a subject having or suffering from multiple myeloma, the method comprising:
administering to the to the subject a viral vector comprising a nucleic acid encoding a fusion protein comprising:
(a) an antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and
(b) a polypeptide antigen comprising a second multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the first multiple myeloma antigen is different from the second multiple myeloma antigen;
wherein the subject (i) previously received ACT (e.g., CAR-T cell therapy), that binds to a cancer cell expressing the second multiple myeloma antigen, (ii) previously exhibited at least one beneficial response to the ACT (e.g., CAR-T cell therapy), and (iii) prior to administration of the fusion protein, the subject exhibits at least one nonbeneficial response to the ACT (e.g., CAR-T cell therapy).

47. The method of claim 46, wherein the fusion protein comprises 2 or more antigen binding polypeptides.

48. The method of claim 47, wherein the fusion protein comprises 2 antigen binding polypeptides.

49. The method of claim 48, wherein the fusion protein comprises:
(a) a first antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA;
(b) a second antigen binding polypeptide that binds a second multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and
(c) a polypeptide antigen comprising of a third multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the first, second, and third multiple myeloma antigens are different.

50. The method of claim 48, wherein the fusion protein comprises:
(a) a first antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA;
(b) a second antigen binding polypeptide that binds a second multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and
(c) a polypeptide antigen comprising of a third multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the third multiple myeloma antigen is different than the first and second.

51. The method of claim 50, wherein the first multiple myeloma antigen and the second multiple myeloma antigen are the same.

52. The method of claim 50, wherein the first multiple myeloma antigen and the second multiple myeloma antigen are CD38.

53. The method of claim 51 or 52, wherein the first and second antigen binding polypeptide are the same, and the fusion protein comprises two copies of the same antigen binding polypeptide.

54. The method of any one of claims 51-53, wherein the first and second antigen binding polypeptide bind to the first and second multiple myeloma antigen at a Kd of about 50 nM to about 2 µM.

55. The method of any one of claims 51-53, wherein the fusion protein binds to a tumor cell expressing the first and second multiple myeloma antigen (e.g., CD38) with higher avidity relative to a healthy or non-tumor cell.

56. The method of claim 55, wherein the fusion protein binds to the tumor cell at a Kd of about 1 to about 40 nM.

57. The method of claim 49, wherein the first multiple myeloma antigen is CD38 and the second multiple myeloma antigen is GPRC5D.

58. A method of treating a subject having or suffering from multiple myeloma, the method comprising:
administering to the subject a fusion protein comprising:
(a) an antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and
(b) a polypeptide antigen comprising a second multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the first multiple myeloma antigen and the second multiple myeloma antigen are different;
wherein the subject is receiving or will receive ACT (e.g., CAR-T cell therapy) for the treatment of multiple myeloma.

59. The method of claim 58, wherein the fusion protein is administered to the subject prior to the subject receiving ACT.

60. The method of claim 58, wherein the fusion protein is administered concurrently with ACT.

61. The method of claim 58, wherein the method further comprises measuring a level of expression of the second multiple myeloma antigen, e.g., in a sample from the subject (e.g., a biological sample, e.g., a tumor sample).

62. The method of claim 61, wherein the method of treatment is continued as long as the subject exhibits at least one beneficial response to the ACT.

63. The method of any one of claims 58-62, wherein the fusion protein comprises 2 or more antigen binding polypeptides.

64. The method of claim 63, wherein the fusion protein comprises 2 antigen binding polypeptides.

65. The method of claim 64, wherein the fusion protein comprises:
(a) a first antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA;
(b) a second antigen binding polypeptide that binds a second multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and
(c) a polypeptide antigen comprising of a third multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the first, second, and third multiple myeloma antigens are different.

66. The method of claim 64, wherein the fusion protein comprises:
(a) a first antigen binding polypeptide that binds a first multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA;
(b) a second antigen binding polypeptide that binds a second multiple myeloma antigen selected from the group consisting of CD38; CS1/SLAMF7; GPRC5D; CD208 (LAMP3); CD307e (FCRL5); ITGA8; ITGB7; CD138; CD272; CD229; CD48; CD150; CD86; CD200; BAFF-R (TNFRSF13C); Tn (GalNAcα1-O-Ser/Thr); sialyl-Tn (STn) (NeuAcα2-6-GalNAcα1-O-Ser/Thr); and BCMA; and
(c) a polypeptide antigen comprising of a third multiple myeloma antigen selected from the group consisting of BCMA, CD38, SLAMF7, CD208, CD307e, CD272; CD229; CD48; CD150; CD86; CD200; BAFF-R (TNFRSF13C); and CD138, wherein the third multiple myeloma antigen is different than the first and second.

67. The method of claim 66, wherein the first multiple myeloma antigen and the second multiple myeloma antigen are the same.

68. The method of claim 66, wherein the first multiple myeloma antigen and the second multiple myeloma antigen are CD38.

69. The method of claim 67 or 68, wherein the first and second antigen binding polypeptide are the same, and the fusion protein comprises two copies of the same antigen binding polypeptide.

70. The method of any one of claims 67-69, wherein the first and second antigen binding polypeptide bind to the first and second multiple myeloma antigen at a Kd of about 50 nM to about 2 µM.

71. The method of any one of claims 67-70 wherein the fusion protein binds to a tumor cell expressing the first and second multiple myeloma antigen (e.g., CD38) with higher avidity relative to a healthy or non-tumor cell.

72. The method of claim 71, wherein the fusion protein binds to the tumor cell at a Kd of about 1 to about 40 nM.

73. The method of claim 58, wherein the first multiple myeloma antigen is CD38 and the second multiple myeloma antigen is GPRC5D.


SEQUENCE LISTING (61 sequences)

SEQ ID NO: 1 - DNA, 1608 bp, Artificial Sequence
  Description of Artificial Sequence: Synthetic polynucleotide
atggagacagacacactcctgctatgggtactgctgctctgggttccaggttccactggtgacatcgtgctgacccagagcccccccagcctggccatgagcctgggcaagagggccaccatcagctgcagggccagcgagagcgtgaccatcctgggcagccacctgatccactggtaccagcagaagcccggccagccccccaccctgctgatccagctggccagcaacgtgcagaccggcgtgcccgccaggttcagcggcagcggcagcaggaccgacttcaccctgaccatcgaccccgtggaggaggacgacgtggccgtgtactactgcctgcagagcaggaccatccccaggaccttcggcggcggcaccaagctggagatcaagggcagcaccagcggcagcggcaagcccggcagcggcgagggcagcaccaagggccagatccagctggtgcagagcggccccgagctgaagaagcccggcgagaccgtgaagatcagctgcaaggccagcggctacaccttcaccgactacagcatcaactgggtgaagagggcccccggcaagggcctgaagtggatgggctggatcaacaccgagaccagggagcccgcctacgcctacgacttcaggggcaggttcgccttcagcctggagaccagcgccagcaccgcctacctgcagatcaacaacctgaagtacgaggacaccgccacctacttctgcgccctggactacagctacgccatggactactggggccagggcaccagcgtgaccgtgagcagcgactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtccgcagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagccctgccccctcgc

SEQ ID NO: 2 - DNA, 1038 bp, Artificial Sequence
  Description of Artificial Sequence: Synthetic polynucleotide
atgaggctgctggtgctgctgtggggctgcctgctgctgcccggctacgaggccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgccggcggcggcggcagcggcggcggcggcagcggcggcggcggcagcggcggcggcggatccgacatccagatgacccagagccccagcagcctgagcgccagcgtgggcgacagggtgaccatcacctgcagggccagccagggcatcaggagctggctggcctggtaccagcagaagcccgagaaggcccccaagagcctgatctacgccgccagcagcctgcagagcggcgtgcccagcaggttcagcggcagcggcagcggcaccgacttcaccctgaccatcagcagcctgcagcccgaggacttcgccacctactactgccagcagtacaacagctaccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 3 - DNA, 1038 bp, Artificial Sequence
  Description of Artificial Sequence: Synthetic polynucleotide
atgaggctgctggtgctgctgtggggctgcctgctgctgcccggctacgaggccgacatccagatgacccagagccccagcagcctgagcgccagcgtgggcgacagggtgaccatcacctgcagggccagccagggcatcaggagctggctggcctggtaccagcagaagcccgagaaggcccccaagagcctgatctacgccgccagcagcctgcagagcggcgtgcccagcaggttcagcggcagcggcagcggcaccgacttcaccctgaccatcagcagcctgcagcccgaggacttcgccacctactactgccagcagtacaacagctaccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggcggcggcagcggcggcggcggcagcggcggcggcggcagcggcggcggcggatccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgcccaccaccaccaccaccac

SEQ ID NO: 4 - DNA, 816 bp, Artificial Sequence
  Description of Artificial Sequence: Synthetic polynucleotide
atggagaccgacacactgctgctgtgggtgctgctgctgtgggtgcccggcagcacaggacagtccgtgctgacccagccagcctccgtgtctggcagcccaggccagtctctgaccatcagctgcaccggcacatctaacgatgtgggcgcctacaagtatgtgagctggtatcagcagtatcccggcaaggcccctaagctgatcctgtacgacgtgttcaagaggccttccggcgtgtctaaccgcttttccggctctaagagcgataatacagcctccctgaccatctctggactgcaggcagaggacgaggcagattactattgcttcagcctgacaagctccaacacctacgtgtttggcaccggcacaaaggtgaccgtgctgggctcccggggcggaggaggcagcggaggaggaggctccggcggcggcggctctctggagatggcacagatgcagctggtgcagagcggagcagaggtgaagaagcctggagccagcgtgaaggtgtcctgtaaggcctctggctacacattcaatcggtatgccatcacctgggtgagacaggcaccaggacagggcctggagtggatgggctggatcagcgcctacaacggcaattcccactatgcccagaagctgcagggccgggtgacaatgaccacagacacctccacaggcaccgcctacatggagctgcggagactgaggtctgacgataccgccgtgtactattgtgcccgcatggcctatgattcctggggccagggcacactggtgaccgtgtctagccaccaccaccaccaccac

SEQ ID NO: 5 - DNA, 825 bp, Artificial Sequence
  Description of Artificial Sequence: Synthetic polynucleotide
atggagaccgacacactgctgctgtgggtgctgctgctgtgggtgcccggctctaccggacagagcgtgctgacacagccaccttccgcctctggcacccctggacagcgggtgacaatcagctgctccggcagccggagcaacgtgggaggcaattacgtgttctggtatcagcaggtgccaggagcaaccccaaagctgctgatctaccggtccaaccagagaccttctggcgtgccagatcggtttgcaggctccaagtctggcagctccgcctccctggccatctctggactgagaagcgaggacgaggccgattactattgcgccacctgggacgatagcctgtccggcttcgtgtttggcaccggcacaaaggtgacagtgctgggcagccggggcggaggaggcagcggaggaggaggctccggcggcggcggctctctggagatggccgaggtgcagctggtggagtccggaggaggactggtgaagccaggaggctccctgaggctgtcttgtgcagccagcggcttcacctttagcgactactatatgtcctggatcagacaggcaccaggcaagggcctggagtgggtgtcttacatctctagctccggcagcaccatctactatgccgactccgtgaagggcaggttcacaatctctcgcgataacgccaagaatagcctgtatctgcagatgaattccctgagggccgaggacacagccgtgtactattgtgccagaggctacggcaaggcctatgatcagtggggccagggcaccctggtgacagtgtctagccaccaccaccaccaccac

SEQ ID NO: 6 - DNA, 828 bp, Artificial Sequence
  Description of Artificial Sequence: Synthetic polynucleotide
atggagaccgatacactgctgctgtgggtgctgctgctgtgggtgcccggcagcaccggcagctccgagctgacacaggaccccgccgtgtccgtggccctgggacagaccgtgaggatcacatgccagggcgacagcctgcgctcctactatgccagctggtatcagcagaagccaggacaggcacccgtgctggtcatctacggcaagaacaataggccttctggcatcccagatcgcttcagcggctctagctccggcaacaccgcctctctgaccatcacaggagcacaggcagaggacgaggcagattactattgtaactccagggactctagcggcaatccccctgtggtgtttggaggaggcaccaagctgacagtgctgggcagccgcggcggaggaggctctggaggaggaggcagcggcggcggcggctccctggagatggcccaggtgcagctggtggagtccggaggaggactggtgcacccaggaggctctctgaggctgagctgcgcagcctccggcttcacctttcggtcccactctatgaactgggtgagacaggcaccaggcaagggcctggagtgggtgtcctctatcagctccgactccacctacacatactatgccgattctgtgaagggccggttcaccatctccagagacaacgccaagaattctctgtatctgcagatgaatagcctgcgggccgaggatacagccgtgtactattgtgccagaagcggcggccagtggaagtactatgactactggggccagggcaccctggtgacagtgtctagccaccaccaccaccaccac

SEQ ID NO: 7 - DNA, 828 bp, Artificial Sequence
  Description of Artificial Sequence: Synthetic polynucleotide
atggagaccgacacactgctgctgtgggtgctgctgctgtgggtgcccggctccaccggacagtctgtggtgacacagccacctagcatgtccgccgcacctggacagcaggtgaccatctcttgcagcggcggcaactccaatatcgagaggaactacgtgtcttggtatctgcagctgccaggcacagcccccaagctggtcatcttcgacaatgatcggagacctagcggcatcccagaccgcttttccggctctaagagcggcacctccgccacactgggaatcaccggactgcagacaggcgacgaggcagattactattgcggcacctgggatagctccctgaggggatgggtgttcggaggaggcaccaagctgacagtgctgggctcccgcggcggaggaggctctggaggaggaggcagcggcggcggcggctccctggagatggccgaggtgcagctggtggagtccggaggaggactgatccagccaggaggcagcctgaggctgtcctgtgcagcctctggcttcacctttagcaactacgcaatgaattgggtgcggcaggcaccaggcaagggcctggagtgggtgtctaccatcaacggcagaggctctagcacaatctatgccgacagcgtgaagggccggtttaccatcagcagagataactccaagaatacactgtacctgcagatgaatagcctgagagccgaggacaccgccacatactattgtgccaggtatatctctcgcggcctgggcgatagctggggacagggcaccctggtgacagtgtcctctcaccaccaccaccaccac

SEQ ID NO: 8 - DNA, 1008 bp, Artificial Sequence
  Description of Artificial Sequence: Synthetic polynucleotide
atggagaccgacacactgctgctgtgggtgctgctgctgtgggtgcccggcagcacaggacagtccgtgctgacccagccagcctccgtgtctggcagcccaggccagtctctgaccatcagctgcaccggcacatctaacgatgtgggcgcctacaagtatgtgagctggtatcagcagtatcccggcaaggcccctaagctgatcctgtacgacgtgttcaagaggccttccggcgtgtctaaccgcttttccggctctaagagcgataatacagcctccctgaccatctctggactgcaggcagaggacgaggcagattactattgcttcagcctgacaagctccaacacctacgtgtttggcaccggcacaaaggtgaccgtgctgggctcccggggcggaggaggcagcggaggaggaggctccggcggcggcggctctctggagatggcacagatgcagctggtgcagagcggagcagaggtgaagaagcctggagccagcgtgaaggtgtcctgtaaggcctctggctacacattcaatcggtatgccatcacctgggtgagacaggcaccaggacagggcctggagtggatgggctggatcagcgcctacaacggcaattcccactatgcccagaagctgcagggccgggtgacaatgaccacagacacctccacaggcaccgcctacatggagctgcggagactgaggtctgacgataccgccgtgtactattgtgcccgcatggcctatgattcctggggccagggcacactggtgaccgtgtctagcggcggcggcggcagcggcggcggcggatccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgcccaccaccaccaccaccac

SEQ ID NO: 9 - DNA, 1014 bp, Artificial Sequence
  Description of Artificial Sequence: Synthetic polynucleotide
atggagaccgacacactgctgctgtgggtgctgctgctgtgggtgcccggctctaccggacagagcgtgctgacacagccaccttccgcctctggcacccctggacagcgggtgacaatcagctgctccggcagccggagcaacgtgggaggcaattacgtgttctggtatcagcaggtgccaggagcaaccccaaagctgctgatctaccggtccaaccagagaccttctggcgtgccagatcggtttgcaggctccaagtctggcagctccgcctccctggccatctctggactgagaagcgaggacgaggccgattactattgcgccacctgggacgatagcctgtccggcttcgtgtttggcaccggcacaaaggtgacagtgctgggcagccggggcggaggaggcagcggaggaggaggctccggcggcggcggctctctggagatggccgaggtgcagctggtggagtccggaggaggactggtgaagccaggaggctccctgaggctgtcttgtgcagccagcggcttcacctttagcgactactatatgtcctggatcagacaggcaccaggcaagggcctggagtgggtgtcttacatctctagctccggcagcaccatctactatgccgactccgtgaagggcaggttcacaatctctcgcgataacgccaagaatagcctgtatctgcagatgaattccctgagggccgaggacacagccgtgtactattgtgccagaggctacggcaaggcctatgatcagtggggccagggcaccctggtgacagtgtctagcggcggcggcggcagcggcggatccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgcccaccaccaccaccaccactga

SEQ ID NO: 10 - DNA, 1035 bp, Artificial Sequence
  Description of Artificial Sequence: Synthetic polynucleotide
atggagaccgatacactgctgctgtgggtgctgctgctgtgggtgcccggcagcaccggcagctccgagctgacacaggaccccgccgtgtccgtggccctgggacagaccgtgaggatcacatgccagggcgacagcctgcgctcctactatgccagctggtatcagcagaagccaggacaggcacccgtgctggtcatctacggcaagaacaataggccttctggcatcccagatcgcttcagcggctctagctccggcaacaccgcctctctgaccatcacaggagcacaggcagaggacgaggcagattactattgtaactccagggactctagcggcaatccccctgtggtgtttggaggaggcaccaagctgacagtgctgggcagccgcggcggaggaggctctggaggaggaggcagcggcggcggcggctccctggagatggcccaggtgcagctggtggagtccggaggaggactggtgcacccaggaggctctctgaggctgagctgcgcagcctccggcttcacctttcggtcccactctatgaactgggtgagacaggcaccaggcaagggcctggagtgggtgtcctctatcagctccgactccacctacacatactatgccgattctgtgaagggccggttcaccatctccagagacaacgccaagaattctctgtatctgcagatgaatagcctgcgggccgaggatacagccgtgtactattgtgccagaagcggcggccagtggaagtactatgactactggggccagggcaccctggtgacagtgtctagcggcggcggcggcagcggcggcggcggcagcggcggcggcggatccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgcccaccaccaccaccaccac

SEQ ID NO: 11 - DNA, 1035 bp, Artificial Sequence
  Description of Artificial Sequence: Synthetic polynucleotide
atggagaccgacacactgctgctgtgggtgctgctgctgtgggtgcccggctccaccggacagtctgtggtgacacagccacctagcatgtccgccgcacctggacagcaggtgaccatctcttgcagcggcggcaactccaatatcgagaggaactacgtgtcttggtatctgcagctgccaggcacagcccccaagctggtcatcttcgacaatgatcggagacctagcggcatcccagaccgcttttccggctctaagagcggcacctccgccacactgggaatcaccggactgcagacaggcgacgaggcagattactattgcggcacctgggatagctccctgaggggatgggtgttcggaggaggcaccaagctgacagtgctgggctcccgcggcggaggaggctctggaggaggaggcagcggcggcggcggctccctggagatggccgaggtgcagctggtggagtccggaggaggactgatccagccaggaggcagcctgaggctgtcctgtgcagcctctggcttcacctttagcaactacgcaatgaattgggtgcggcaggcaccaggcaagggcctggagtgggtgtctaccatcaacggcagaggctctagcacaatctatgccgacagcgtgaagggccggtttaccatcagcagagataactccaagaatacactgtacctgcagatgaatagcctgagagccgaggacaccgccacatactattgtgccaggtatatctctcgcggcctgggcgatagctggggacagggcaccctggtgacagtgtcctctggcggcggcggcagcggcggcggcggcagcggcggcggcggatccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgcccaccaccaccaccaccac

SEQ ID NO: 12 - protein, 536 aa, Artificial Sequence
  Description of Artificial Sequence: Synthetic polypeptide
METDTLLLWVLLLWVPGSTGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSDYKDDDDKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - protein, 346 aa, Artificial Sequence
  Description of Artificial Sequence: Synthetic polypeptide
MRLLVLLWGCLLLPGYEAMLQMAGQCSQNEYFDSLLHACIPCQLRCSSNTPPLTCQRYCNASVTNSVKGTNAGGGGSGGGGSGGGGSGGGGSDIQMTQSPSSLSASVGDRVTITCRASQGIRSWLAWYQQKPEKAPKSLIYAASSLQSGVPSRFSGSGSGTDFTLTISSLQPEDFATYYCQQYNSYPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 14 - protein, 346 aa, Artificial Sequence
  Description of Artificial Sequence: Synthetic polypeptide
MRLLVLLWGCLLLPGYEADIQMTQSPSSLSASVGDRVTITCRASQGIRSWLAWYQQKPEKAPKSLIYAASSLQSGVPSRFSGSGSGTDFTLTISSLQPEDFATYYCQQYNSYPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSMLQMAGQCSQNEYFDSLLHACIPCQLRCSSNTPPLTCQRYCNASVTNSVKGTNAHHHHHH

SEQ ID NO: 15 - protein, 272 aa, Artificial Sequence
  Description of Artificial Sequence: Synthetic polypeptide
METDTLLLWVLLLWVPGSTGQSVLTQPASVSGSPGQSLTISCTGTSNDVGAYKYVSWYQQYPGKAPKLILYDVFKRPSGVSNRFSGSKSDNTASLTISGLQAEDEADYYCFSLTSSNTYVFGTGTKVTVLGSRGGGGSGGGGSGGGGSLEMAQMQLVQSGAEVKKPGASVKVSCKASGYTFNRYAITWVRQAPGQGLEWMGWISAYNGNSHYAQKLQGRVTMTTDTSTGTAYMELRRLRSDDTAVYYCARMAYDSWGQGTLVTVSSHHHHHH

SEQ ID NO: 16 - protein, 275 aa, Artificial Sequence
  Description of Artificial Sequence: Synthetic polypeptide
METDTLLLWVLLLWVPGSTGQSVLTQPPSASGTPGQRVTISCSGSRSNVGGNYVFWYQQVPGATPKLLIYRSNQRPSGVPDRFAGSKSGSSASLAISGLRSEDEADYYCATWDDSLSGFVFGTGTKVTVLGSRGGGGSGGGGSGGGGSLEMAEVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGYGKAYDQWGQGTLVTVSSHHHHHH

SEQ ID NO: 17 - protein, 276 aa, Artificial Sequence
  Description of Artificial Sequence: Synthetic polypeptide
METDTLLLWVLLLWVPGSTGSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSHHHHHH

SEQ ID NO: 18 - protein, 276 aa, Artificial Sequence
  Description of Artificial Sequence: Synthetic polypeptide
METDTLLLWVLLLWVPGSTGQSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLGSRGGGGSGGGGSGGGGSLEMAEVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTVSSHHHHHH

SEQ ID NO: 19 - protein, 336 aa, Artificial Sequence
  Description of Artificial Sequence: Synthetic polypeptide
METDTLLLWVLLLWVPGSTGQSVLTQPASVSGSPGQSLTISCTGTSNDVGAYKYVSWYQQYPGKAPKLILYDVFKRPSGVSNRFSGSKSDNTASLTISGLQAEDEADYYCFSLTSSNTYVFGTGTKVTVLGSRGGGGSGGGGSGGGGSLEMAQMQLVQSGAEVKKPGASVKVSCKASGYTFNRYAITWVRQAPGQGLEWMGWISAYNGNSHYAQKLQGRVTMTTDTSTGTAYMELRRLRSDDTAVYYCARMAYDSWGQGTLVTVSSGGGGSGGGGSMLQMAGQCSQNEYFDSLLHACIPCQLRCSSNTPPLTCQRYCNASVTNSVKGTNAHHHHHH

SEQ ID NO: 20 - protein, 337 aa, Artificial Sequence
  Description of Artificial Sequence: Synthetic polypeptide
METDTLLLWVLLLWVPGSTGQSVLTQPPSASGTPGQRVTISCSGSRSNVGGNYVFWYQQVPGATPKLLIYRSNQRPSGVPDRFAGSKSGSSASLAISGLRSEDEADYYCATWDDSLSGFVFGTGTKVTVLGSRGGGGSGGGGSGGGGSLEMAEVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGYGKAYDQWGQGTLVTVSSGGGGSGGSMLQMAGQCSQNEYFDSLLHACIPCQLRCSSNTPPLTCQRYCNASVTNSVKGTNAHHHHHH

SEQ ID NO: 21 - protein, 345 aa, Artificial Sequence
  Description of Artificial Sequence: Synthetic polypeptide
METDTLLLWVLLLWVPGSTGSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGGGGSGGGGSGGGGSMLQMAGQCSQNEYFDSLLHACIPCQLRCSSNTPPLTCQRYCNASVTNSVKGTNAHHHHHH

SEQ ID NO: 22 - protein, 345 aa, Artificial Sequence
  Description of Artificial Sequence: Synthetic polypeptide
METDTLLLWVLLLWVPGSTGQSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLGSRGGGGSGGGGSGGGGSLEMAEVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTVSSGGGGSGGGGSGGGGSMLQMAGQCSQNEYFDSLLHACIPCQLRCSSNTPPLTCQRYCNASVTNSVKGTNAHHHHHH

SEQ ID NO: 23 - protein, 54 aa, Artificial Sequence
  Description of Artificial Sequence: Synthetic polypeptide
MLQMAGQCSQNEYFDSLLHACIPCQLRCSSNTPPLTCQRYCNASVTNSVKGTNA

SEQ ID NO: 24 - protein, 5 aa, Artificial Sequence
  Description of Artificial Sequence: Synthetic peptide
GGGGS

SEQ ID NO: 25 - protein, 272 aa, Artificial Sequence
  Description of Artificial Sequence: Synthetic polypeptide
MRLLVLLWGCLLLPGYEADIQMTQSPSSLSASVGDRVTITCRASQGISNYLAWFQQKPGKAPKSLIYAASSLQSGVPSRFSGSGSGTDFTLTISSLQPEDFATYYCQQYNSYPITFGQGTRLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 26 - DNA, 816 bp, Artificial Sequence
  Description of Artificial Sequence: Synthetic polynucleotide
atgaggctgctggtgctgctgtggggctgcctgctgctgcccggctacgaggccgacatccagatgacccagagccccagcagcctgagcgccagcgtgggcgacagggtgaccatcacctgcagggccagccagggcatcagcaactacctggcctggttccagcagaagcccggcaaggcccccaagagcctgatctacgccgccagcagcctgcagagcggcgtgcccagcaggttcagcggcagcggcagcggcaccgacttcaccctgaccatcagcagcctgcagcccgaggacttcgccacctactactgccagcagtacaacagctaccccatcaccttcggccagggcaccaggctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 27 - protein, 272 aa, Artificial Sequence
  Description of Artificial Sequence: Synthetic polypeptide
MRLLVLLWGCLLLPGYEADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 28 - DNA, 816 bp, Artificial Sequence
  Description of Artificial Sequence: Synthetic polynucleotide
atgaggctgctggtgctgctgtggggctgcctgctgctgcccggctacgaggccgacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcacccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 29 - protein, 272 aa, Artificial Sequence
  Description of Artificial Sequence: Synthetic polypeptide
MRLLVLLWGCLLLPGYEAEIVLTQSPDFQSVTPKEKVTITCRASQSIGSSLHWYQQKPDQSPKLLIKYASQSFSGVPSRFSGSGSGTDFTLTINSLEAEDAATYYCHQSSSLPYTFGQGTKLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 30 - DNA, 816 bp, Artificial Sequence
  Description of Artificial Sequence: Synthetic polynucleotide
atgaggctgctggtgctgctgtggggctgcctgctgctgcccggctacgaggccgagatcgtgctgacccagagccccgacttccagagcgtgacccccaaggagaaggtgaccatcacctgcagggccagccagagcatcggcagcagcctgcactggtaccagcagaagcccgaccagagccccaagctgctgatcaagtacgccagccagagcttcagcggcgtgcccagcaggttcagcggcagcggcagcggcaccgacttcaccctgaccatcaacagcctggaggccgaggacgccgccacctactactgccaccagagcagcagcctgccctacaccttcggccagggcaccaagctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 31 - protein, 272 aa, Artificial Sequence
  Description of Artificial Sequence: Synthetic polypeptide
MRLLVLLWGCLLLPGYEAAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 32 - DNA, 816 bp, Artificial Sequence
  Description of Artificial Sequence: Synthetic polynucleotide
atgaggctgctggtgctgctgtggggctgcctgctgctgcccggctacgaggccgccatccagctgacccagagccccagcagcctgagcgccagcgtgggcgacagggtgaccatcacctgcagggccagccagggcatcagcagcgccctggcctggtaccagcagaagcccggcaaggcccccaagctgctgatctacgacgccagcagcctggagagcggcgtgcccagcaggttcagcggcagcggcagcggcaccgacttcaccctgaccatcagcagcctgcagcccgaggacttcgccacctactactgccagcagttcaacagctaccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 33 - protein, 540 aa, Artificial Sequence
  Description of Artificial Sequence: Synthetic polypeptide
MRLLVLLWGCLLLPGYEADIQMTQSPSSLSASVGDRVTITCRASQGISNYLAWFQQKPGKAPKSLIYAASSLQSGVPSRFSGSGSGTDFTLTISSLQPEDFATYYCQQYNSYPITFGQGTRLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSDIQMTQSPSSLSASVGDRVTITCRASQGISNYLAWFQQKPGKAPKSLIYAASSLQSGVPSRFSGSGSGTDFTLTISSLQPEDFATYYCQQYNSYPITFGQGTRLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 34 - DNA, 1620 bp, Artificial Sequence
  Description of Artificial Sequence: Synthetic polynucleotide
atgaggctgctggtgctgctgtggggctgcctgctgctgcccggctacgaggccgacatccagatgacccagagccccagcagcctgagcgccagcgtgggcgacagggtgaccatcacctgcagggccagccagggcatcagcaactacctggcctggttccagcagaagcccggcaaggcccccaagagcctgatctacgccgccagcagcctgcagagcggcgtgcccagcaggttcagcggcagcggcagcggcaccgacttcaccctgaccatcagcagcctgcagcccgaggacttcgccacctactactgccagcagtacaacagctaccccatcaccttcggccagggcaccaggctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgacatccagatgacccagagccccagcagcctgagcgccagcgtgggcgacagggtgaccatcacctgcagggccagccagggcatcagcaactacctggcctggttccagcagaagcccggcaaggcccccaagagcctgatctacgccgccagcagcctgcagagcggcgtgcccagcaggttcagcggcagcggcagcggcaccgacttcaccctgaccatcagcagcctgcagcccgaggacttcgccacctactactgccagcagtacaacagctaccccatcaccttcggccagggcaccaggctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 35 - protein, 540 aa, Artificial Sequence
  Description of Artificial Sequence: Synthetic polypeptide
MRLLVLLWGCLLLPGYEADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 36 - DNA, 1620 bp, Artificial Sequence
  Description of Artificial Sequence: Synthetic polynucleotide
atgaggctgctggtgctgctgtggggctgcctgctgctgcccggctacgaggccgacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcacccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcacccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 37 - protein, 540 aa, Artificial Sequence
  Description of Artificial Sequence: Synthetic polypeptide
MRLLVLLWGCLLLPGYEAEIVLTQSPDFQSVTPKEKVTITCRASQSIGSSLHWYQQKPDQSPKLLIKYASQSFSGVPSRFSGSGSGTDFTLTINSLEAEDAATYYCHQSSSLPYTFGQGTKLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSEIVLTQSPDFQSVTPKEKVTITCRASQSIGSSLHWYQQKPDQSPKLLIKYASQSFSGVPSRFSGSGSGTDFTLTINSLEAEDAATYYCHQSSSLPYTFGQGTKLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 38 - DNA, 1620 bp, Artificial Sequence
  Description of Artificial Sequence: Synthetic polynucleotide
atgaggctgctggtgctgctgtggggctgcctgctgctgcccggctacgaggccgagatcgtgctgacccagagccccgacttccagagcgtgacccccaaggagaaggtgaccatcacctgcagggccagccagagcatcggcagcagcctgcactggtaccagcagaagcccgaccagagccccaagctgctgatcaagtacgccagccagagcttcagcggcgtgcccagcaggttcagcggcagcggcagcggcaccgacttcaccctgaccatcaacagcctggaggccgaggacgccgccacctactactgccaccagagcagcagcctgccctacaccttcggccagggcaccaagctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgagatcgtgctgacccagagccccgacttccagagcgtgacccccaaggagaaggtgaccatcacctgcagggccagccagagcatcggcagcagcctgcactggtaccagcagaagcccgaccagagccccaagctgctgatcaagtacgccagccagagcttcagcggcgtgcccagcaggttcagcggcagcggcagcggcaccgacttcaccctgaccatcaacagcctggaggccgaggacgccgccacctactactgccaccagagcagcagcctgccctacaccttcggccagggcaccaagctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 39 - protein, 540 aa, Artificial Sequence
  Description of Artificial Sequence: Synthetic polypeptide
MRLLVLLWGCLLLPGYEAAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 40 - DNA, 1620 bp, Artificial Sequence
  Description of Artificial Sequence: Synthetic polynucleotide
atgaggctgctggtgctgctgtggggctgcctgctgctgcccggctacgaggccgccatccagctgacccagagccccagcagcctgagcgccagcgtgggcgacagggtgaccatcacctgcagggccagccagggcatcagcagcgccctggcctggtaccagcagaagcccggcaaggcccccaagctgctgatctacgacgccagcagcctggagagcggcgtgcccagcaggttcagcggcagcggcagcggcaccgacttcaccctgaccatcagcagcctgcagcccgaggacttcgccacctactactgccagcagttcaacagctaccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgccatccagctgacccagagccccagcagcctgagcgccagcgtgggcgacagggtgaccatcacctgcagggccagccagggcatcagcagcgccctggcctggtaccagcagaagcccggcaaggcccccaagctgctgatctacgacgccagcagcctggagagcggcgtgcccagcaggttcagcggcagcggcagcggcaccgacttcaccctgaccatcagcagcctgcagcccgaggacttcgccacctactactgccagcagttcaacagctaccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 41 - protein, 614 aa, Artificial Sequence
  Description of Artificial Sequence: Synthetic polypeptide
MRLLVLLWGCLLLPGYEADIQMTQSPSSLSASVGDRVTITCRASQGISNYLAWFQQKPGKAPKSLIYAASSLQSGVPSRFSGSGSGTDFTLTISSLQPEDFATYYCQQYNSYPITFGQGTRLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSDIQMTQSPSSLSASVGDRVTITCRASQGISNYLAWFQQKPGKAPKSLIYAASSLQSGVPSRFSGSGSGTDFTLTISSLQPEDFATYYCQQYNSYPITFGQGTRLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSMLQMAGQCSQNEYFDSLLHACIPCQLRCSSNTPPLTCQRYCNASVTNSVKGTNAHHHHHH

SEQ ID NO: 42 - DNA, 1842 bp, Artificial Sequence
  Description of Artificial Sequence: Synthetic polynucleotide
atgaggctgctggtgctgctgtggggctgcctgctgctgcccggctacgaggccgacatccagatgacccagagccccagcagcctgagcgccagcgtgggcgacagggtgaccatcacctgcagggccagccagggcatcagcaactacctggcctggttccagcagaagcccggcaaggcccccaagagcctgatctacgccgccagcagcctgcagagcggcgtgcccagcaggttcagcggcagcggcagcggcaccgacttcaccctgaccatcagcagcctgcagcccgaggacttcgccacctactactgccagcagtacaacagctaccccatcaccttcggccagggcaccaggctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgacatccagatgacccagagccccagcagcctgagcgccagcgtgggcgacagggtgaccatcacctgcagggccagccagggcatcagcaactacctggcctggttccagcagaagcccggcaaggcccccaagagcctgatctacgccgccagcagcctgcagagcggcgtgcccagcaggttcagcggcagcggcagcggcaccgacttcaccctgaccatcagcagcctgcagcccgaggacttcgccacctactactgccagcagtacaacagctaccccatcaccttcggccagggcaccaggctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggcggcggcagcggcggcggcggcagcggcggcggcggcagcggcggcggcggatccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgcccaccaccaccaccaccac

SEQ ID NO: 43 - protein, 614 aa, Artificial Sequence
  Description of Artificial Sequence: Synthetic polypeptide
MRLLVLLWGCLLLPGYEAMLQMAGQCSQNEYFDSLLHACIPCQLRCSSNTPPLTCQRYCNASVTNSVKGTNAGGGGSGGGGSGGGGSGGGGSDIQMTQSPSSLSASVGDRVTITCRASQGISNYLAWFQQKPGKAPKSLIYAASSLQSGVPSRFSGSGSGTDFTLTISSLQPEDFATYYCQQYNSYPITFGQGTRLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSDIQMTQSPSSLSASVGDRVTITCRASQGISNYLAWFQQKPGKAPKSLIYAASSLQSGVPSRFSGSGSGTDFTLTISSLQPEDFATYYCQQYNSYPITFGQGTRLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 44 - DNA, 1842 bp, Artificial Sequence
  Description of Artificial Sequence: Synthetic polynucleotide
atgaggctgctggtgctgctgtggggctgcctgctgctgcccggctacgaggccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgccggcggaggcggatccggcggcggcggcagcggtggcggaggctccggcggaggaggcagcgacatccagatgacccagagccccagcagcctgagcgccagcgtgggcgacagggtgaccatcacctgcagggccagccagggcatcagcaactacctggcctggttccagcagaagcccggcaaggcccccaagagcctgatctacgccgccagcagcctgcagagcggcgtgcccagcaggttcagcggcagcggcagcggcaccgacttcaccctgaccatcagcagcctgcagcccgaggacttcgccacctactactgccagcagtacaacagctaccccatcaccttcggccagggcaccaggctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgacatccagatgacccagagccccagcagcctgagcgccagcgtgggcgacagggtgaccatcacctgcagggccagccagggcatcagcaactacctggcctggttccagcagaagcccggcaaggcccccaagagcctgatctacgccgccagcagcctgcagagcggcgtgcccagcaggttcagcggcagcggcagcggcaccgacttcaccctgaccatcagcagcctgcagcccgaggacttcgccacctactactgccagcagtacaacagctaccccatcaccttcggccagggcaccaggctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 45 - protein, 614 aa, Artificial Sequence
  Description of Artificial Sequence: Synthetic polypeptide
MRLLVLLWGCLLLPGYEADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSMLQMAGQCSQNEYFDSLLHACIPCQLRCSSNTPPLTCQRYCNASVTNSVKGTNAHHHHHH

SEQ ID NO: 46 - DNA, 1842 bp, Artificial Sequence
  Description of Artificial Sequence: Synthetic polynucleotide
atgaggctgctggtgctgctgtggggctgcctgctgctgcccggctacgaggccgacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcacccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcacccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggcggcggcagcggcggcggcggcagcggcggcggcggcagcggcggcggcggatccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgcccaccaccaccaccaccac

SEQ ID NO: 47 - protein, 614 aa, Artificial Sequence
  Description of Artificial Sequence: Synthetic polypeptide
MRLLVLLWGCLLLPGYEAMLQMAGQCSQNEYFDSLLHACIPCQLRCSSNTPPLTCQRYCNASVTNSVKGTNAGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 48 - DNA, 1842 bp, Artificial Sequence
  Description of Artificial Sequence: Synthetic polynucleotide
atgaggctgctggtgctgctgtggggctgcctgctgctgcccggctacgaggccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgccggcggaggcggatccggcggcggcggcagcggtggcggaggctccggcggaggaggcagcgacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcacccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcacccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 49 - protein, 614 aa, Artificial Sequence
  Description of Artificial Sequence: Synthetic polypeptide
MRLLVLLWGCLLLPGYEAEIVLTQSPDFQSVTPKEKVTITCRASQSIGSSLHWYQQKPDQSPKLLIKYASQSFSGVPSRFSGSGSGTDFTLTINSLEAEDAATYYCHQSSSLPYTFGQGTKLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSEIVLTQSPDFQSVTPKEKVTITCRASQSIGSSLHWYQQKPDQSPKLLIKYASQSFSGVPSRFSGSGSGTDFTLTINSLEAEDAATYYCHQSSSLPYTFGQGTKLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSMLQMAGQCSQNEYFDSLLHACIPCQLRCSSNTPPLTCQRYCNASVTNSVKGTNAHHHHHH

SEQ ID NO: 50 - DNA, 1842 bp, Artificial Sequence
  Description of Artificial Sequence: Synthetic polynucleotide
atgaggctgctggtgctgctgtggggctgcctgctgctgcccggctacgaggccgagatcgtgctgacccagagccccgacttccagagcgtgacccccaaggagaaggtgaccatcacctgcagggccagccagagcatcggcagcagcctgcactggtaccagcagaagcccgaccagagccccaagctgctgatcaagtacgccagccagagcttcagcggcgtgcccagcaggttcagcggcagcggcagcggcaccgacttcaccctgaccatcaacagcctggaggccgaggacgccgccacctactactgccaccagagcagcagcctgccctacaccttcggccagggcaccaagctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgagatcgtgctgacccagagccccgacttccagagcgtgacccccaaggagaaggtgaccatcacctgcagggccagccagagcatcggcagcagcctgcactggtaccagcagaagcccgaccagagccccaagctgctgatcaagtacgccagccagagcttcagcggcgtgcccagcaggttcagcggcagcggcagcggcaccgacttcaccctgaccatcaacagcctggaggccgaggacgccgccacctactactgccaccagagcagcagcctgccctacaccttcggccagggcaccaagctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggcggcggcagcggcggcggcggcagcggcggcggcggcagcggcggcggcggatccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgcccaccaccaccaccaccac

SEQ ID NO: 51 - protein, 614 aa, Artificial Sequence
  Description of Artificial Sequence: Synthetic polypeptide
MRLLVLLWGCLLLPGYEAMLQMAGQCSQNEYFDSLLHACIPCQLRCSSNTPPLTCQRYCNASVTNSVKGTNAGGGGSGGGGSGGGGSGGGGSEIVLTQSPDFQSVTPKEKVTITCRASQSIGSSLHWYQQKPDQSPKLLIKYASQSFSGVPSRFSGSGSGTDFTLTINSLEAEDAATYYCHQSSSLPYTFGQGTKLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSEIVLTQSPDFQSVTPKEKVTITCRASQSIGSSLHWYQQKPDQSPKLLIKYASQSFSGVPSRFSGSGSGTDFTLTINSLEAEDAATYYCHQSSSLPYTFGQGTKLEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 52 - DNA, 1842 bp, Artificial Sequence
  Description of Artificial Sequence: Synthetic polynucleotide
atgaggctgctggtgctgctgtggggctgcctgctgctgcccggctacgaggccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgccggcggaggcggatccggcggcggcggcagcggtggcggaggctccggcggaggaggcagcgagatcgtgctgacccagagccccgacttccagagcgtgacccccaaggagaaggtgaccatcacctgcagggccagccagagcatcggcagcagcctgcactggtaccagcagaagcccgaccagagccccaagctgctgatcaagtacgccagccagagcttcagcggcgtgcccagcaggttcagcggcagcggcagcggcaccgacttcaccctgaccatcaacagcctggaggccgaggacgccgccacctactactgccaccagagcagcagcctgccctacaccttcggccagggcaccaagctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgagatcgtgctgacccagagccccgacttccagagcgtgacccccaaggagaaggtgaccatcacctgcagggccagccagagcatcggcagcagcctgcactggtaccagcagaagcccgaccagagccccaagctgctgatcaagtacgccagccagagcttcagcggcgtgcccagcaggttcagcggcagcggcagcggcaccgacttcaccctgaccatcaacagcctggaggccgaggacgccgccacctactactgccaccagagcagcagcctgccctacaccttcggccagggcaccaagctggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 53 - protein, 614 aa, Artificial Sequence
  Description of Artificial Sequence: Synthetic polypeptide
MRLLVLLWGCLLLPGYEAAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSMLQMAGQCSQNEYFDSLLHACIPCQLRCSSNTPPLTCQRYCNASVTNSVKGTNAHHHHHH

SEQ ID NO: 54 - DNA, 1842 bp, Artificial Sequence
  Description of Artificial Sequence: Synthetic polynucleotide
atgaggctgctggtgctgctgtggggctgcctgctgctgcccggctacgaggccgccatccagctgacccagagccccagcagcctgagcgccagcgtgggcgacagggtgaccatcacctgcagggccagccagggcatcagcagcgccctggcctggtaccagcagaagcccggcaaggcccccaagctgctgatctacgacgccagcagcctggagagcggcgtgcccagcaggttcagcggcagcggcagcggcaccgacttcaccctgaccatcagcagcctgcagcccgaggacttcgccacctactactgccagcagttcaacagctaccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgccatccagctgacccagagccccagcagcctgagcgccagcgtgggcgacagggtgaccatcacctgcagggccagccagggcatcagcagcgccctggcctggtaccagcagaagcccggcaaggcccccaagctgctgatctacgacgccagcagcctggagagcggcgtgcccagcaggttcagcggcagcggcagcggcaccgacttcaccctgaccatcagcagcctgcagcccgaggacttcgccacctactactgccagcagttcaacagctaccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggcggcggcagcggcggcggcggcagcggcggcggcggcagcggcggcggcggatccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgcccaccaccaccaccaccac

SEQ ID NO: 55 - protein, 614 aa, Artificial Sequence
  Description of Artificial Sequence: Synthetic polypeptide
MRLLVLLWGCLLLPGYEAMLQMAGQCSQNEYFDSLLHACIPCQLRCSSNTPPLTCQRYCNASVTNSVKGTNAGGGGSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSGGGGSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSGGGGSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSSHHHHHH

SEQ ID NO: 56 - DNA, 1842 bp, Artificial Sequence
  Description of Artificial Sequence: Synthetic polynucleotide
atgaggctgctggtgctgctgtggggctgcctgctgctgcccggctacgaggccatgctgcagatggccggccagtgcagccagaacgagtacttcgacagcctgctgcacgcctgcatcccctgccagctgaggtgcagcagcaacaccccccccctgacctgccagaggtactgcaacgccagcgtgaccaacagcgtgaagggcaccaacgccggcggaggcggatccggcggcggcggcagcggtggcggaggctccggcggaggaggcagcgccatccagctgacccagagccccagcagcctgagcgccagcgtgggcgacagggtgaccatcacctgcagggccagccagggcatcagcagcgccctggcctggtaccagcagaagcccggcaaggcccccaagctgctgatctacgacgccagcagcctggagagcggcgtgcccagcaggttcagcggcagcggcagcggcaccgacttcaccctgaccatcagcagcctgcagcccgaggacttcgccacctactactgccagcagttcaacagctaccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagcggcggaggcggatccggcggcggcggcagcggtggcggaggctccggcggaggaggcagcgccatccagctgacccagagccccagcagcctgagcgccagcgtgggcgacagggtgaccatcacctgcagggccagccagggcatcagcagcgccctggcctggtaccagcagaagcccggcaaggcccccaagctgctgatctacgacgccagcagcctggagagcggcgtgcccagcaggttcagcggcagcggcagcggcaccgacttcaccctgaccatcagcagcctgcagcccgaggacttcgccacctactactgccagcagttcaacagctaccccctgaccttcggcggcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccttcggcggcaccttcagcagctacgccatcagctgggtgaggcaggcccccggccagggcctggagtggatgggcaggatcatcaggttcctgggcatcgccaactacgcccagaagttccagggcagggtgaccctgatcgccgacaagagcaccaacaccgcctacatggagctgagcagcctgaggagcgaggacaccgccgtgtactactgcgccggcgagcccggcgagagggaccccgacgccgtggacatctggggccagggcaccatggtgaccgtgagcagccaccaccaccaccaccac

SEQ ID NO: 57 - protein, 121 aa, Artificial Sequence
  Description of Artificial Sequence: Synthetic polypeptide
QVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSS

SEQ ID NO: 58 - protein, 107 aa, Artificial Sequence
  Description of Artificial Sequence: Synthetic polypeptide
DIQMTQSPSSLSASVGDRVTITCRASQGIRSWLAWYQQKPEKAPKSLIYAASSLQSGVPSRFSGSGSGTDFTLTISSLQPEDFATYYCQQYNSYPLTFGGGTKVEIK

SEQ ID NO: 59 - protein, 25 aa, Artificial Sequence
  Description of Artificial Sequence: Synthetic peptide
  SITE (1)..(25): This sequence may encompass 2-5 "Gly Gly Gly Gly Ser" repeating units
GGGGSGGGGSGGGGSGGGGSGGGGS

SEQ ID NO: 60 - protein, 20 aa, Artificial Sequence
  Description of Artificial Sequence: Synthetic peptide
GGGGSGGGGSGGGGSGGGGS

SEQ ID NO: 61 - protein, 18 aa, Artificial Sequence
  Description of Artificial Sequence: Synthetic peptide
GSTSGSGKPGSGEGSTKG